(19) United States
(12) Patent Application Publication
(10) Pub. No.: US 2024/0390317 A1
(43) Pub. Date: Nov. 28, 2024

(54) COMBINATION TREATMENT AND/OR PREVENTION OF CARDIAC DISEASES IN NON-HUMAN MAMMALS COMPRISING ONE OR MORE SGLT-2 INHIBITORS AND PIMOBENDAN AND/OR TELMISARTAN

(71) Applicant: BOEHRINGER INGELHEIM VETMEDICA GMBH, Ingelheim am Rhein (DE)

(72) Inventors: Carla KROH, Oestrich-Winkel (DE); Ingo Ulrich LANG, Ingelheim am Rhein (DE); José MATALLO, Frankfurt am Main (DE); Tanja Margrit ZIMMERING, Ingelheim (DE)

(21) Appl. No.: 18/666,911

(22) Filed: May 17, 2024

(30) Foreign Application Priority Data
May 24, 2023 (EP) 23175197.5

(51) Int. Cl. A61K 31/351; A61K 31/4184; A61K 31/501; A61P 9/10

(52) U.S. Cl. CPC A61K 31/351; A61K 31/4184; A61K 31/501; A61P 9/10

(57) ABSTRACT

The present invention is directed to the use of one or more SGLT-2 inhibitors or pharmaceutically acceptable forms thereof in combination with pimobendan and/or telmisartan or pharmaceutically acceptable forms thereof, in particular for the prophylaxis and/or treatment of one or more cardiac diseases in a non-human mammal/non-human mammal patient, such as a dog or a cat.

### FIELD OF THE INVENTION

The invention relates to the field of medicine, in particular to the field of veterinary medicine. The invention relates to the combination treatment and/or prevention of one or more cardiac diseases in a non-human mammal, in particular a dog or a cat, comprising one or more SGLT-2 inhibitors or a pharmaceutically acceptable form thereof and pimobendan and/or telmisartan or a pharmaceutically acceptable form thereof.

### BACKGROUND INFORMATION

Recently, SGLT-2 inhibitors were shown to reduce the risk for hospitalisation for heart failure and the risk of new onset of heart failure events in human patients with type II diabetes. A program called EMPEROR was initiated recently to investigate if empagliflozin shows favourable effects in human patients with heart disease independent of diabetes. It was recently announced that the EMPEROR-Reduced Phase III trial as part of the EMPEROR program showed that empagliflozin reduced the risk for the composite endpoint of cardiovascular death or hospitalization due to heart failure in adults with heart failure and reduced or preserved ejection fraction, with and without diabetes.

To date, SGLT-2 inhibitors have been used in clinical trials on different subsets of human patients suffering from heart failure. However, the pathology of cardiac disease in dogs and cats differs significantly to the pathology observed in humans, where e.g., arteriosclerosis, which is not reported in dogs or cats, is the major concern. Cardiomyopathies are not a common cardiovascular disease in people. Also, they were defined as exclusion criteria for trial participation (McMurray J J V et al., Eur. J. Heart Fail. 2019; 21:665-675: see page 667, table 1, item 9.).

State of the art interventions are based on specific action on unique pathways for symptomatic treatment of secondary conditions induced by cardiac disease, such as positive inotropics (improved contraction), ACE inhibitors, Angiotensin receptor blocker (reduce high blood pressure), and diuretics (increased fluid excretion). A known pharmaceutically active compound to treat heart failure is pimobendan (4,5-dihydro-6-[2-(4-methoxyphenyl)-1H-benzimidazol-5-yl]-5-methyl-3(2H)-pyridazinone) disclosed in EP 0 008 391 and having the formula:

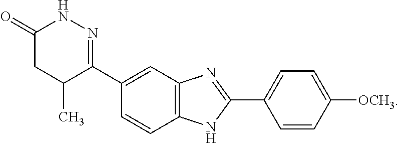 [SMILES: COc1ccc(-c2nc3cc(C4=NNC(=O)CC4C)ccc3[nH]2)cc1]

Pimobendan is a well-known compound for the treatment of congestive heart failure (CHF) originating for example from dilated cardiomyopathy (DCM) or mitral valve disease (MVD) in animals, especially dogs. Pimobendan is also approved as a drug product for cardiovascular treatment in humans in Japan.

A known pharmaceutically active compound to treat systemic diseases, such as chronic kidney disease, is the angiotensin II receptor antagonist telmisartan (4′-[2-n-propyl-4-methyl-6-(1-methylbenzimidazol-2-yl)-benzimidazol-1-ylmethyl]-biphenyl-2-carboxylic acid), which was developed for the treatment of hypertension and other medical indications as disclosed in EP 0 502 314 and having the formula:

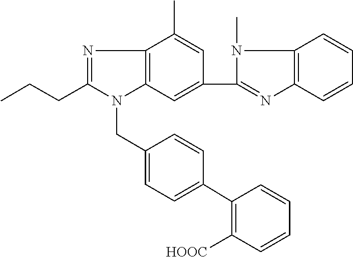 [SMILES: CCCc1nc2c(C)cc(-c3nc4ccccc4n3C)cc2n1Cc1ccc(-c2ccccc2C(=O)O)cc1]

Telmisartan is already sold on the market under the trade name Micardis® (Boehringer Ingelheim, Germany) for treatment/prophylaxis of humans. It exists in two polymorphic forms as disclosed in WO 00/043370, U.S. Pat. Nos. 6,358,986 and 6,410,742. Sodium salts of telmisartan and its solvate, hydrate, and hemihydrate are disclosed in WO 2003/037876.

Canine (myxomatous) mitral-valve disease [(M)MVD] and dilated cardiomyopathy (DCM) are the most common cardiovascular conditions in dogs and the most frequent cause of canine heart failure. Additionally, aortic stenosis, typically caused by a ridge or ring of fibrotic tissue in the subaortic region (subaortic stenosis), is a common congenital defect of large breed dogs. Those conditions have specific pathophysiology, but are characterized by a reduced pump capacity, increased muscular effort and energy imbalance, finally resulting in heart failure. In people, beneficial cardiovascular effects under the use of SGLT-2 inhibitors have been observed. However, the direct effect on the heart remains unknown. Also, the pathology of cardiac disease in humans (coronary disease, stroke, infarction) differs significantly from the pathologies observed in dogs [(M)MVD, DCM]. The American College of Veterinary Internal Medicine (ACVIM) consensus statement provides specific classification and treatment references for (M)MVD. Although the guideline targets (M)MVD the classifications are commonly used for other cardiac diseases, like DCM. The reference defines the different stages of canine cardiac disease as below:
Stage A: dogs are at higher-than-average risk for developing heart failure, but without any apparent structural abnormality (i.e., no audible heart murmur) at the time of examination.
Stage B: dogs in Stage B have a structural abnormality [e.g., the presence of (M)MVD], but have never had clinical signs of heart failure associated with their disease. Stage B is divided into:
Stage B1: describes asymptomatic dogs that have no radiographic or echocardiographic evidence of cardiac remodeling in response to their (M)MVD, as well as those in which remodeling changes are present, but not severe enough to meet current clinical trial criteria that have been used to determine that initiating treatment is warranted.
Stage B2: refers to asymptomatic dogs that have more advanced mitral valve regurgitation that is hemodynamically severe and long-standing enough to have caused radiographic and echocardiographic findings of left atrial and ventricular enlargement that meet clinical trial criteria used to identify dogs that clearly should benefit from initiating pharmacologic treatment to delay the onset of heart failure.
Stage C: dogs have (M)MVD severe enough to cause current or past clinical signs of heart failure. Stage C includes all dogs with (M)MVD that have experienced an episode of clinical heart failure and that are not refractory to standard heart failure treatment. These patients continue to be categorized as Stage C even after improvement or complete resolution of their clinical signs with standard treatment. In exceptional cases that undergo successful surgical mitral valve repair, reclassification to Stage B is warranted.
Stage D: refers to dogs with end-stage (M)MVD, in which clinical signs of heart failure are refractory to standard treatment (defined later in this consensus statement). Such patients require advanced or specialized treatment strategies to remain clinically comfortable with their disease, and at some point, treatment efforts become futile without surgical repair of the valve. As with Stage C, the panel has distinguished between dogs in Stage D that require acute, hospital-based treatment and those that can be managed as outpatients.

Standard treatment is usually recommended as of stage B1 in order to slow progression of the disease, clinical treatment is clearly needed as of stage B2. Management of heart failure is palliative and is aimed at controlling clinical signs related to the presence of oedema and cavity effusion. These are accomplished through reducing preload and/or afterload by diuretics and vasodilators, improving cardiac performance (positive inotropes, positive lusitropes, antiarrhythmics), and using neurohormonal modulators (ACE inhibitors, and potentially β-blockers, aldosterone antagonists, and angiotensin II receptor blockers).

Heart disease is one of the most common diseases of pet cats, affecting 10-15% of all cats (Freeman et al., Cardiol Res. 2017, 8 (4): 139-142; Payne JR et al., J Vet Cardiol. 2015, 17 (Suppl1): S244-S257).

Feline heart diseases are classified in congenital and acquired heart diseases. The majority of heart diseases are chronic, incurable and progress over time. After a sub-clinical stage, clinical signs of heart failure and ultimately cardiac death may occur. Typical symptoms for a heart disease are: poor general condition, weakness, lethargy, depression, anorexia, tachycardia, tachypnea, dyspnoea, congestion, oedema, a low peripheral blood pressure and acute posterior paresis or paralysis. Cardiomyopathies are the most common heart diseases in cats. They are classified in primary cardiomyopathies (Hypertrophic Cardiomyopathy (HC/HCM/HOCM), Restrictive Cardiomyopathy (RCM), Unclassified Cardiomyopathy (UCM), Arrhythmogenic Right Ventricular Cardiomyopathy (ARVC) and Dilated Cardiomyopathy (DCM), which is very rare in cats) and in secondary cardiomyopathies due to nutritional disorders (taurine deficiency), metabolic disorders (hyperthyroidism, acromegaly), infiltrative processes (neoplasia, amyloidosis) and inflammatory processes (toxins, immune reactions, infectious agents). The classification of the cardiomyopathy is based on echocardiographic measurements. Cats are most commonly affected by Hypertrophic Cardiomyopathy (HCM), with a prevalence of 10-15% in the general pet cat population. However, due to an inheritant form, breeds such as the Maine Coon cat, Persian, Ragdoll, and Sphynx are at higher risk.

Although heart disease is common in cats, atherosclerosis, which is a major risk factor for the development of heart failure in humans, is notably absent in cats. This is related, at least in part, to the fact that these species have high high-density lipoprotein concentrations (Freeman et al., Cardiol Res. 2017, 8 (4): 139-142). In contrast to humans, in cats cardiomyopathies are thought to be the main reason for feline heart failure although not much seems to be known about the etiology of the different forms of disease.

Clinical studies have shown a median survival time in cats with HCM ranging from 92 to 2,153 days, depending on the predominant clinical signs of the population studied (i.e., asymptomatic vs. congestive heart failure (CHF) vs. arterial thromboembolism (ATE) (Atkins C E et al., J Am Vet Med Assoc. 1992, 201 (4): 613-618: Rush JE et al., J Am Vet Med Assoc. 2002, 220 (2): 202-207; Payne JR et al., J Vet Intern Med. 2013, 27 (6): 1427-1436). Reported median survival times for cats with HCM and heart failure, for example, range from only 92 to 563 days.

Hoenig M et al. (J Vet Pharmacol Therapeutics 2018, 41 (2): 266-273) discloses the effects of SGLT-2 inhibitor velagliflozin with the therapeutic potential to treat diabetes in cats.

Lin Y et al. (J Am Heart Assoc 2021, 10: e019274) discloses that dapagliflozin improves cardiac hemodynamics and mitigates arrhythmogenesis in mitral regurgitation-induced myocardial dysfunction.

Little C J L et al., (J Small Anim Prac 2008, 49 (1): 17-25) discloses that heart failure is common in diabetic cats: findings from a retrospective case-controlled study in first-opinion practice.

Matsumura K et al. (Cardiovascular Ultrasound 2019, 17 (1): 26) discloses the effect of SGLT-2 inhibitors on cardiac function and cardiovascular outcome.

Nishinarity R et al. (J Am Heart Assoc 2021, 10: e017483) discloses that canagliflozin suppresses atrial remodeling in a canine atrial fibrillation model.

Santos-Gallego C G et al. (J American College Cardiol 2019, 73 (15): 1931-1944) discloses that empagliflozin ameliorates adverse left ventricular remodeling in non-diabetic heart failure by enhancing myocardial energetics.

Silva Custodio Jr J et al. (Heart Failure Reviews 2018, 23 (3): 409-418) discloses SGLT-2 inhibition and heart failure current concepts.

US 2011/098240 discloses a pharmaceutical composition comprising a SGLT-2 inhibitor in combination with a DPP IV inhibitor, which is suitable in the treatment or prevention of one or more conditions selected from type 1 diabetes mellitus, type 2 diabetes mellitus, impaired glucose tolerance and hyperglycemia.

US 2015/164856 discloses one or more SGLT2 inhibitors or pharmaceutically acceptable forms thereof for use in the treatment and/or prevention of a metabolic disorder in a feline animal, preferably wherein the metabolic disorder is one or more selected from the group consisting of: ketoacidosis, pre-diabetes, diabetes mellitus type 1 or type 2, insulin resistance, obesity, hyperglycemia, impaired glucose tolerance, hyperinsulinemia, dyslipidemia, dysadipokinemia, subclinical inflammation, systemic inflammation, low grade systemic inflammation, hepatic lipidosis, atherosclerosis, inflammation of the pancreas, neuropathy and/or Syndrome X (metabolic syndrome) and/or loss of pancreatic beta cell function and/or wherein the remission of the metabolic disorder, preferably diabetic remission, is achieved and/or maintained.

US 2016/000816 discloses certain SGLT-2 inhibitors for treating and/or preventing oxidative stress, for example in human patients with type 1 or type 2 diabetes, as well as to the use of such SGLT-2 inhibitors in treatment and/or prevention of cardiovascular diseases in human patients, for example type 1 or type 2 diabetes patients.

US 2017/266152 discloses methods for preventing or treating acute or chronic heart failure and for reducing the risk of cardiovascular death, hospitalization for heart failure and other conditions in human patients with preserved or reduced ejection fraction by administering empagliflozin to the patient.

US 2019/076395 discloses the use of certain SGLT-2 inhibitors, such as ertugliflozin or a pharmaceutically acceptable salt or a co-crystal thereof, for treating, reducing the risk of and/or preventing heart failure, myocardial infarction, cardiovascular disease or cardiovascular death in animals without type 2 or type 1 diabetes mellitus, or in animals with pre-diabetes, or in animals with type 2 or type 1 diabetes mellitus or pre-diabetes.

U.S. Pat. No. 10,537,570 discloses the use of pimobendan in a method of reducing the heart size and/or delaying the onset of clinical symptoms in a patient suffering from asymptomatic (occult, preclinical) heart failure, due to mitral valve disease.

WO 2005/092343 describes the use of PDE-III inhibitors, such as pimobendan, for the reduction of heart size of a patient suffering from heart failure without, however, mentioning patients with asymptomatic (occult, preclinical) heart failure due to mitral valve disease (MVD).

WO 2007/054514 is directed to the use of PDE-III inhibitors, such as pimobendan, for the treatment of asymptomatic (also known as occult or preclinical) heart failure without, however, mentioning patients with asymptomatic (occult, preclinical) heart failure due to mitral valve disease (MVD).

WO 2011/153953 discloses crystalline forms of benzylbenzene SGLT-2 inhibitors and mentions among others the treatment of chronic heart failure in humans.

WO 2017/174571 discloses pimobendan for use in a method of reducing the heart size and/or delaying the onset of clinical symptoms in a patient suffering from asymptomatic (occult, preclinical) heart failure, preferably congestive heart failure, due to mitral valve disease (MVD), and/or delaying the onset of heart failure, preferably congestive heart failure, in a patient suffering from asymptomatic (occult, preclinical) heart failure, preferably congestive heart failure, due to mitral valve disease (MVD), wherein the patient is preferably a mammal, more preferably a human, a dog, a cat or a horse, and most preferably a dog.

WO 2019/059557/US 2020/054656 discloses a pharmaceutical composition comprising an SGLT-2 inhibitor and a therapeutic agent for treating hypertension. WO 2021/092341/US 2023/000816 discloses sodium-glucose linked transporter inhibitors for the management of chronic kidney disease, hypertension and heart failure in companion animals.

WO 2021/165177/US 2021/260090 discloses the use of one or more SGLT-2 inhibitors or pharmaceutically acceptable forms thereof for the prophylaxis and/or treatment of one or more cardiac diseases in feline animals.

WO 2022/036506 discloses a fixed dose combination of a compositions of SGLT-2 inhibitors and angiotensin receptor blockers.

WO 2023/006718 discloses the use of one or more SGLT-2 inhibitors or pharmaceutically acceptable forms thereof for the prophylaxis and/or treatment of one or more cardiac diseases in a non-human mammal/non-human mammal patient excluding a feline, in particular a canine/canine patient.

Notwithstanding the disclosures of the above documents, there is a medical need for the combination treatment and/or prevention of cardiac diseases in a non-human mammal (patient), in particular a dog (patient) or a cat (patient).

### SUMMARY OF THE INVENTION

The present invention concerns one or more SGLT-2 inhibitors or pharmaceutically acceptable forms thereof in combination with pimobendan and/or telmisartan or pharmaceutically acceptable forms thereof for use as a medicament.

In one aspect, the present invention also concerns the one or more SGLT-2 inhibitors or pharmaceutically acceptable forms thereof in combination with pimobendan and/or telmisartan or pharmaceutically acceptable forms thereof for use as herein disclosed and/or claimed in a method of prevention and/or treatment of one or more cardiac diseases in a non-human mammal/non-human mammal patient, in particular a canine/canine patient or a feline/feline patient.

A corresponding method of preventing and/or treating one or more cardiac diseases in a non-human mammal/non-human mammal patient, in particular a canine/canine patient or a feline/feline patient, comprising administering one or more SGLT-2 inhibitors or pharmaceutically acceptable forms thereof in combination with pimobendan and/or telmisartan or pharmaceutically acceptable forms thereof to such non-human mammal/non-human mammal patient as well as the corresponding use of one or more SGLT-2 inhibitors or pharmaceutically acceptable forms thereof in combination with pimobendan and/or telmisartan or pharmaceutically acceptable forms thereof for the preparation of a medicament for the prevention and/or treatment of one or more cardiac diseases in a non-human mammal/non-human mammal patient are also intended to be comprised by the present invention.

In another aspect, the present invention also concerns the one or more SGLT-2 inhibitors or pharmaceutically acceptable forms thereof in combination with pimobendan and/or telmisartan or pharmaceutically acceptable forms thereof for use as herein disclosed and/or claimed in a method of treatment of one or more cardiac diseases in a non-human mammal/non-human mammal patient, in particular a canine/canine patient or a feline/feline patient.

A corresponding method of treating one or more cardiac diseases in a non-human mammal/non-human mammal patient, in particular a canine/canine patient or a feline/feline patient, comprising administering one or more SGLT-2 inhibitors or pharmaceutically acceptable forms thereof in combination with pimobendan and/or telmisartan or pharmaceutically acceptable forms thereof to such non-human mammal/non-human mammal patient as well as the corresponding use of one or more SGLT-2 inhibitors or pharmaceutically acceptable forms thereof in combination with pimobendan and/or telmisartan or pharmaceutically acceptable forms thereof for the preparation of a medicament for the treatment of one or more cardiac diseases in a non-human mammal/non-human mammal patient are also intended to be comprised by the present invention.

In yet another aspect, the present invention also concerns the one or more SGLT-2 inhibitors or pharmaceutically acceptable forms thereof in combination with pimobendan and/or telmisartan or pharmaceutically acceptable forms thereof for use as herein disclosed and/or claimed, wherein the one or more cardiac diseases are selected from the group consisting of: acquired cardiac disease; congenital cardiac disease; heart failure; congestive heart failure; asymptomatic/preclinical/occult heart failure; heart failure due to (myxomatous) mitral valve disease [(M)MVD]; congestive heart failure due to (myxomatous) mitral valve disease [(M)MVD]; asymptomatic/preclinical/occult heart failure due to (myxomatous) mitral valve disease [(M)MVD]; (myxomatous) mitral valve disease [(M)MVD]; clinically overt (myxomatous) mitral valve disease [(M)MVD]; asymptomatic/preclinical/occult (myxomatous) mitral valve disease [(M)MVD]; heart failure due to dilated cardiomyopathy (DCM); congestive heart failure due to dilated cardiomyopathy (DCM); asymptomatic/preclinical/occult heart failure due to dilated cardiomyopathy (DCM); dilated cardiomyopathy (DCM); clinically overt dilated cardiomyopathy (DCM); asymptomatic/preclinical/occult dilated cardiomyopathy (DCM); aortic stenosis (valvular, supravalvular and/or subvalvular); heart failure due to one or more cardiomyopathies, heart failure due to hypertrophic cardiomyopathy (HCM), heart failure due to hypertrophic obstructive cardiomyopathy (HOCM), heart failure due to restrictive cardiomyopathy (RCM), heart failure due to dilated cardiomyopathy (DCM), heart failure due to unclassified cardiomyopathy (UCM), heart failure due to arrhythmogenic right ventricular cardiomyopathy (ARVC), hypertrophic cardiomyopathy (HCM), hypertrophic obstructive cardiomyopathy (HOCM), restrictive cardiomyopathy (RCM), dilated cardiomyopathy (DCM), unclassified cardiomyopathy (UCM), and/or arrhythmogenic right ventricular cardiomyopathy (ARVC).

A corresponding method of preventing and/or treating the above exemplified one or more cardiac diseases in a non-human mammal/non-human mammal patient, in particular a canine/canine patient or a feline/feline patient, comprising administering one or more SGLT-2 inhibitors or pharmaceutically acceptable forms thereof in combination with pimobendan and/or telmisartan or pharmaceutically acceptable forms thereof to such non-human mammal/non-human mammal patient as well as the corresponding use of one or more SGLT-2 inhibitors or pharmaceutically acceptable forms thereof in combination with pimobendan and/or telmisartan or pharmaceutically acceptable forms thereof for the preparation of a medicament for the prevention and/or treatment of the above exemplified one or more cardiac diseases in a non-human mammal/non-human mammal patient are also intended to be comprised by the present invention.

In yet another aspect, the present invention also concerns the one or more SGLT-2 inhibitors or pharmaceutically acceptable forms thereof in combination with pimobendan and/or telmisartan or pharmaceutically acceptable forms thereof for use as herein disclosed and/or claimed, wherein the one or more cardiac diseases are selected from the group consisting of: acquired cardiac disease; congenital cardiac disease; heart failure; congestive heart failure; asymptomatic/preclinical/occult heart failure; (myxomatous) mitral valve disease [(M)MVD]; clinically overt (myxomatous) mitral valve disease [(M)MVD]; asymptomatic/preclinical/occult (myxomatous) mitral valve disease [(M)MVD]; dilated cardiomyopathy (DCM); clinically overt dilated cardiomyopathy (DCM); asymptomatic/preclinical/occult dilated cardiomyopathy (DCM), aortic stenosis (valvular, supravalvular and/or subvalvular); and wherein the non-human mammal/non-human mammal patient is a canine/canine patient: preferably a canine patient in need of such prevention and/or treatment; more preferably a dog in need of such prevention and/or treatment, even more preferably a non-diabetic dog in need of such prevention and/or treatment.

A corresponding method of preventing and/or treating the above exemplified one or more cardiac diseases in a canine/canine patient, comprising administering one or more SGLT-2 inhibitors or pharmaceutically acceptable forms thereof in combination with pimobendan and/or telmisartan or pharmaceutically acceptable forms thereof to such canine/canine patient as well as the corresponding use of one or more SGLT-2 inhibitors or pharmaceutically acceptable forms thereof in combination with pimobendan and/or telmisartan or pharmaceutically acceptable forms thereof for the preparation of a medicament for the prevention and/or treatment of the above exemplified one or more cardiac diseases in a canine/canine patient are also intended to be comprised by the present invention.

In yet another aspect, the present invention also concerns the one or more SGLT-2 inhibitors or pharmaceutically acceptable forms thereof in combination with pimobendan and/or telmisartan or pharmaceutically acceptable forms thereof for use as herein disclosed and/or claimed, wherein the one or more cardiac diseases are selected from the group consisting of: (myxomatous) mitral valve disease [(M)MVD]; clinically overt (myxomatous) mitral valve disease [(M)MVD]; asymptomatic/preclinical/occult (myxomatous) mitral valve disease [(M)MVD]; dilated cardiomyopathy (DCM); clinically overt dilated cardiomyopathy (DCM); asymptomatic/preclinical/occult dilated cardiomyopathy (DCM); and wherein the non-human mammal/non-human mammal patient is a canine/canine patient; preferably a canine patient in need of such prevention and/or treatment; more preferably a dog in need of such prevention and/or treatment, even more preferably a non-diabetic dog in need of such prevention and/or treatment.

A corresponding method of preventing and/or treating the above exemplified one or more cardiac diseases in a canine/canine patient, comprising administering one or more SGLT-2 inhibitors or pharmaceutically acceptable forms thereof in combination with pimobendan and/or telmisartan or pharmaceutically acceptable forms thereof to such canine/canine patient as well as the corresponding use of one or more SGLT-2 inhibitors or pharmaceutically acceptable forms thereof in combination with pimobendan and/or telmisartan or pharmaceutically acceptable forms thereof for the preparation of a medicament for the prevention and/or treatment of the above exemplified one or more cardiac diseases in a canine/canine patient are also intended to be comprised by the present invention.

In yet another aspect, the present invention also concerns the one or more SGLT-2 inhibitors or pharmaceutically acceptable forms thereof in combination with pimobendan and/or telmisartan or pharmaceutically acceptable forms thereof for use as herein disclosed and/or claimed, wherein the one or more cardiac diseases are selected from the group consisting of: acquired cardiac disease: congenital cardiac disease; heart failure; congestive heart failure; asymptomatic/preclinical/occult heart failure; heart failure due to one or more cardiomyopathies, heart failure due to hypertrophic cardiomyopathy (HCM), heart failure due to hypertrophic obstructive cardiomyopathy (HOCM), heart failure due to restrictive cardiomyopathy (RCM), heart failure due to dilated cardiomyopathy (DCM), heart failure due to unclassified cardiomyopathy (UCM), heart failure due to arrhythmogenic right ventricular cardiomyopathy (ARVC), hypertrophic cardiomyopathy (HCM), hypertrophic obstructive cardiomyopathy (HOCM), restrictive cardiomyopathy (RCM), dilated cardiomyopathy (DCM), unclassified cardiomyopathy (UCM), and/or arrhythmogenic right ventricular cardiomyopathy (ARVC); and wherein the non-human mammal/non-human mammal patient is a feline/feline patient; preferably a feline patient in need of such prevention and/or treatment, more preferably a cat in need of such prevention and/or treatment, even more preferably a non-diabetic cat in need of such prevention and/or treatment.

A corresponding method of preventing and/or treating the above exemplified one or more cardiac diseases in a feline/feline patient, comprising administering one or more SGLT-2 inhibitors or pharmaceutically acceptable forms thereof in combination with pimobendan and/or telmisartan or pharmaceutically acceptable forms thereof to such feline/feline patient as well as the corresponding use of one or more SGLT-2 inhibitors or pharmaceutically acceptable forms thereof in combination with pimobendan and/or telmisartan or pharmaceutically acceptable forms thereof for the preparation of a medicament for the prevention and/or treatment of the above exemplified one or more cardiac diseases in a feline/feline patient are also intended to be comprised by the present invention.

In yet another aspect, the present invention also concerns the one or more SGLT-2 inhibitors or pharmaceutically acceptable forms thereof in combination with pimobendan and/or telmisartan or pharmaceutically acceptable forms thereof for use as herein disclosed and/or claimed, wherein the one or more cardiac diseases are selected from the group consisting of: heart failure due to hypertrophic cardiomyopathy (HCM); hypertrophic cardiomyopathy (HCM); and wherein the non-human mammal/non-human mammal patient is a feline/feline patient: preferably a feline patient in need of such prevention and/or treatment, more preferably a cat in need of such prevention and/or treatment, even more preferably a non-diabetic cat in need of such prevention and/or treatment.

A corresponding method of preventing and/or treating the above exemplified one or more cardiac diseases in a feline/feline patient, comprising administering one or more SGLT-2 inhibitors or pharmaceutically acceptable forms thereof in combination with pimobendan and/or telmisartan or pharmaceutically acceptable forms thereof to such feline/feline patient as well as the corresponding use of one or more SGLT-2 inhibitors or pharmaceutically acceptable forms thereof in combination with pimobendan and/or telmisartan or pharmaceutically acceptable forms thereof for the preparation of a medicament for the prevention and/or treatment of the above exemplified one or more cardiac diseases in a feline/feline patient are also intended to be comprised by the present invention.

In yet another aspect, the present invention also concerns the following preferred embodiments:

One or more SGLT-2 inhibitors or pharmaceutically acceptable forms thereof in combination with pimobendan and/or telmisartan or pharmaceutically acceptable forms thereof for use in a method of prevention and/or treatment of DCM or MVD in a canine/canine patient.

One or more SGLT-2 inhibitors or pharmaceutically acceptable forms thereof in combination with pimobendan and/or telmisartan or pharmaceutically acceptable forms thereof for use in a method of prevention and/or treatment of HCM in a feline/feline patient.

One or more SGLT-2 inhibitors or pharmaceutically acceptable forms thereof in combination with pimobendan and/or telmisartan or pharmaceutically acceptable forms thereof for use in a method of treatment of DCM or MVD in a canine/canine patient.

One or more SGLT-2 inhibitors or pharmaceutically acceptable forms thereof in combination with pimobendan and/or telmisartan or pharmaceutically acceptable forms thereof for use in a method of treatment of

HCM in a feline/feline patient.

One or more SGLT-2 inhibitors or pharmaceutically acceptable forms thereof in combination with pimobendan for use in a method of treatment of DCM or MVD in a canine/canine patient. One or more SGLT-2 inhibitors or pharmaceutically acceptable forms thereof in combination with pimobendan for use in a method of treatment of HCM in a feline/feline patient.

In yet another aspect, the present invention also concerns the one or more SGLT-2 inhibitors or pharmaceutically acceptable forms thereof in combination with pimobendan and/or telmisartan or pharmaceutically acceptable forms thereof for the uses as herein disclosed and/or claimed, wherein the one or more SGLT-2 inhibitors are selected from the group consisting of:
(1) a glucopyranosyl-substituted benzene derivative of the formula (1)

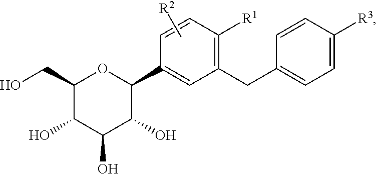

wherein R¹ denotes cyano, Cl or methyl (most preferably cyano);
R² denotes H, methyl, methoxy or hydroxy (most preferably H) and
R³ denotes cyclopropyl, hydrogen, fluorine, chlorine, bromine, iodine, methyl, ethyl, propyl, isopropyl, butyl, sec-butyl, iso-butyl, tert-butyl, 3-methyl-but-1-yl, cyclobutyl, cyclopentyl, cyclohexyl, 1-hydroxy-cyclopropyl, 1-hydroxy-cyclobutyl, 1-hydroxy-cyclopentyl, 1-hydroxy-cyclohexyl, ethinyl, ethoxy, difluoromethyl, trifluoromethyl, pentafluoroethyl, 2-hydroxyl-ethyl, hydroxymethyl, 3-hydroxy-propyl, 2-hydroxy-2-methyl-prop-1-yl, 3-hydroxy-3-methyl-but-1-yl, 1-hydroxy-1-methyl-ethyl, 2,2,2-trifluoro-1-hydroxy-1-methyl-ethyl, 2,2,2-trifluoro-1-hydroxy-1-trifluoromethyl-ethyl, 2-methoxy-ethyl, 2-ethoxy-ethyl, hydroxy, difluoromethyloxy, trifluoromethyloxy, 2-methyloxy-ethyloxy, methylsulfanyl, methylsulfinyl, methlysulfonyl, ethylsulfinyl, ethylsulfonyl, trimethylsilyl, (R)-tetrahydrofuran-3-yloxy or (S)-tetrahydrofuran-3-yloxy or cyano;
wherein R³ is preferably selected from cyclopropyl, ethyl, ethinyl, ethoxy, (R)-tetrahydrofuran-3-yloxy or (S)-tetrahydrofuran-3-yloxy; and most preferably R³ is cyclopropyl, or a derivative thereof wherein one or more hydroxyl groups of the β-D-glucopyranosyl group are acylated with groups selected from (C₁₋₁₈-alkyl)carbonyl, (C₁₋₁₈-alkyl)oxycarbonyl, phenyl-carbonyl and phenyl-(C₁₋₃-alkyl)-carbonyl;
(2) Velagliflozin, represented by formula (2):

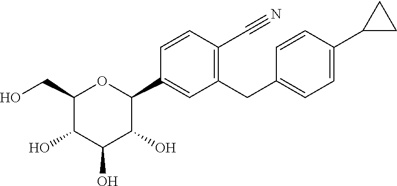 [SMILES: N#Cc1ccc([C@@H]2O[C@H](CO)[C@@H](O)[C@H](O)[C@H]2O)cc1Cc1ccc(C2CC2)cc1]

(3) Dapagliflozin, represented by formula (3):

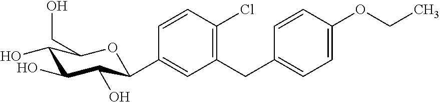

(4) Canagliflozin, represented by formula (4):

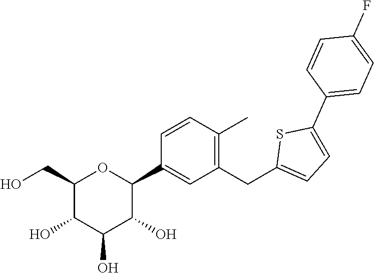 [SMILES: Cc1ccc([C@@H]2O[C@H](CO)[C@@H](O)[C@H](O)[C@H]2O)cc1Cc1ccc(-c2ccc(F)cc2)s1]

(5) Empagliflozin, represented by formula (5):

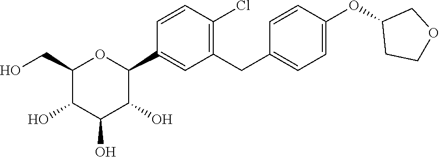 [SMILES: OC[C@H]1O[C@@H](c2ccc(Cl)c(Cc3ccc(O[C@H]4CCOC4)cc3)c2)[C@H](O)[C@@H](O)[C@@H]1O]

(6) I.useogliflozin, represented by formula (6):

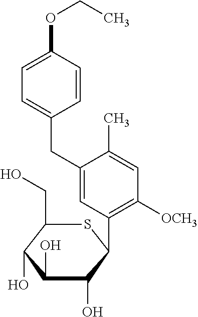

(7) Tofogliflozin, represented by formula (7):

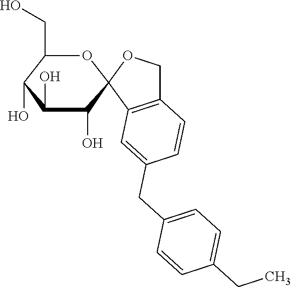

(8) Ipragliflozin, represented by formula (8):

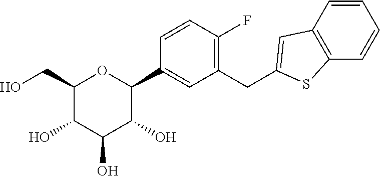 [SMILES: OC[C@H]1O[C@@H](c2ccc(F)c(Cc3cc4ccccc4s3)c2)[C@H](O)[C@@H](O)[C@@H]1O]

(9) Ertugliflozin, represented by formula (9):

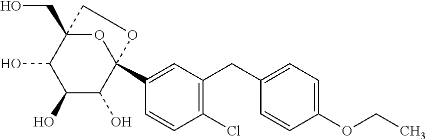 [SMILES: CCOc1ccc(Cc2cc([C@]34OC[C@](CO)(O3)[C@@H](O)[C@H](O)[C@H]4O)ccc2Cl)cc1]

(10) Atigliflozin, represented by formula (10):

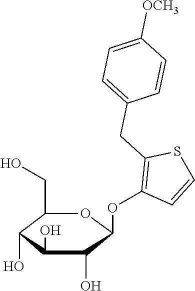

(11) Remogliflozin, represented by formula (11):

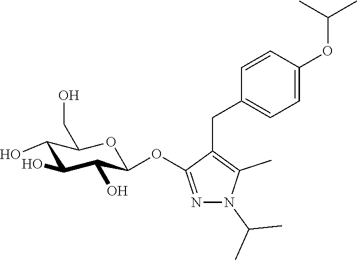

(11A) Remogliflozin etabonate, represented by formula (11A):

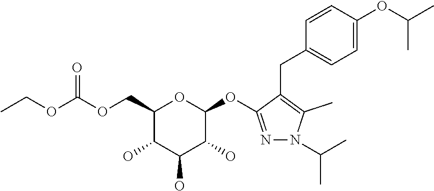 [SMILES: CCOC(=O)OC[C@H]1O[C@@H](Oc2nn(C(C)C)c(C)c2Cc2ccc(OC(C)C)cc2)[C@H](O)[C@@H](O)[C@@H]1O]

(12) a thiophene derivative of the formula (12)

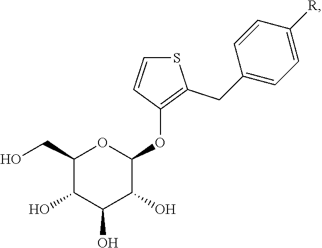

wherein R denotes methoxy or trifluoromethoxy;
(13) 1-(β-D-glucopyranosyl)-4-methyl-3-[5-(4-fluorophenyl)-2-thienylmethyl] benzene, represented by formula (13);

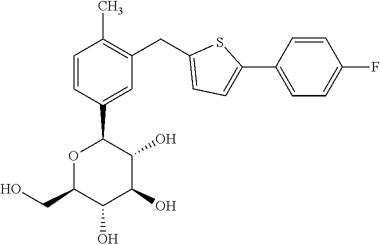 [SMILES: Cc1ccc([C@@H]2O[C@H](CO)[C@@H](O)[C@H](O)[C@H]2O)cc1Cc1ccc(-c2ccc(F)cc2)s1]

(14) a spiroketal derivative of the formula (14):

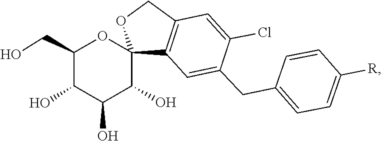

wherein R denotes methoxy, trifluoromethoxy, ethoxy, ethyl, isopropyl or tert. butyl;
(15) a pyrazole-O-glucoside derivative of the formula (15)

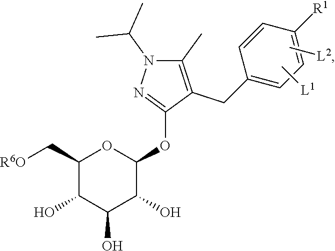

wherein
R¹ denotes C₁₋₃-alkoxy,
L¹, L² independently of each other denote H or F,
R⁶ denotes H, (C₁₋₃-alkyl)carbonyl, (C₁₋₆-alkyl)oxycarbonyl, phenyloxycarbonyl, benzyloxycarbonyl or benzylcarbonyl;
(16) Sotagliflozin, represented by formula (16):

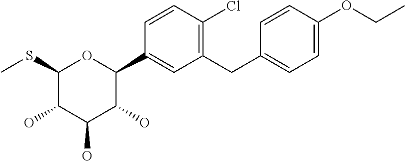 [SMILES: CCOc1ccc(Cc2cc([C@@H]3O[C@H](SC)[C@@H](O)[C@H](O)[C@H]3O)ccc2Cl)cc1]

(17) Sergliflozin, represented by formula (17):

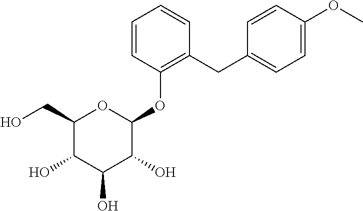 [SMILES: COc1ccc(Cc2ccccc2O[C@@H]2O[C@H](CO)[C@@H](O)[C@H](O)[C@H]2O)cc1]

(18) a compound represented by formula (18):

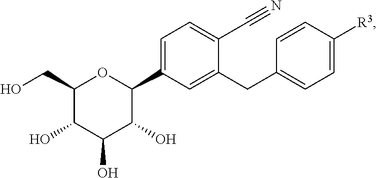

wherein
R³ denotes cyclopropyl, hydrogen, fluorine, chlorine, bromine, iodine, methyl, ethyl, propyl, isopropyl, butyl, sec-butyl, iso-butyl, tert-butyl, 3-methyl-but-1-yl, cyclobutyl, cyclopentyl, cyclohexyl, 1-hydroxy-cyclopropyl, 1-hydroxy-cyclobutyl, 1-hydroxy-cyclopentyl, 1-hydroxy-cyclohexyl, ethinyl, ethoxy, difluoromethyl, trifluoromethyl, pentafluoroethyl, 2-hydroxyl-ethyl, hydroxymethyl, 3-hydroxy-propyl, 2-hydroxy-2-methyl-prop-1-yl, 3-hydroxy-3-methyl-but-1-yl, 1-hydroxy-1-methyl-ethyl, 2,2,2-trifluoro-1-hydroxy-1-methyl-ethyl, 2,2,2-trifluoro-1-hydroxy-1-trifluoromethyl-ethyl, 2-methoxy-ethyl, 2-ethoxy-ethyl, hydroxy, difluoromethyloxy, trifluoromethyloxy, 2-methyloxy-ethyloxy, methylsulfanyl, methylsulfinyl, methlysulfonyl, ethylsulfinyl, ethylsulfonyl, trimethylsilyl, (R)-tetrahydrofuran-3-yloxy or (S)-tetrahydrofuran-3-yloxy or cyano, and wherein R³ is preferably selected from cyclopropyl, ethyl, ethinyl, ethoxy, (R)-tetrahydrofuran-3-yloxy or (S)-tetrahydrofuran-3-yloxy; and R³ most preferably is cyclopropyl,
or a derivative thereof wherein one or more hydroxyl groups of the β-D-glucopyranosyl group are acylated with groups selected from (C₁₋₁₈-alkyl)carbonyl, (C₁₋₁₈-alkyl)oxycarbonyl, phenylcarbonyl and phenyl-(C₁₋₃-alkyl)-carbonyl;
(19) Bexagliflozin, represented by formula (19):

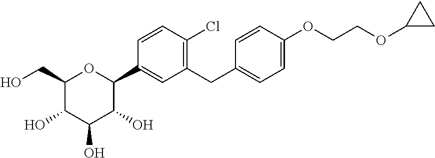 [SMILES: OC[C@H]1O[C@@H](c2ccc(Cl)c(Cc3ccc(OCCOC4CC4)cc3)c2)[C@H](O)[C@@H](O)[C@@H]1O]

(20) Janagliflozin, represented by formula (20):

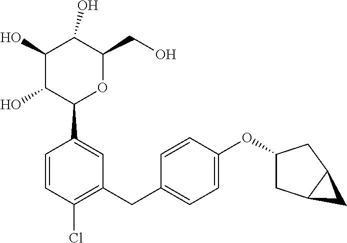 [SMILES: OC[C@H]1O[C@@H](c2ccc(Cl)c(Cc3ccc(O[C@H]4C[C@@H]5C[C@@H]5C4)cc3)c2)[C@H](O)[C@@H](O)[C@@H]1O]

(21) Rongliflozin, represented by formula (21):

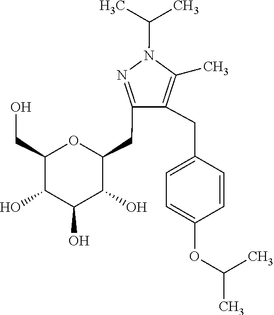 [SMILES: Cc1c(Cc2ccc(OC(C)C)cc2)c(C[C@@H]2O[C@H](CO)[C@@H](O)[C@H](O)[C@H]2O)nn1C(C)C]

(22) Wanpagliflozin;
(23) Enavogliflozin, represented by formula (23):

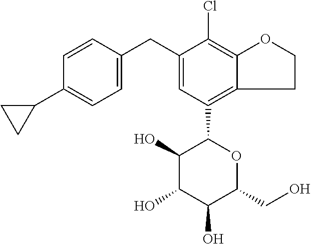 [SMILES: OC[C@H]1O[C@@H](c2cc(Cc3ccc(C4CC4)cc3)c(Cl)c3c2CCO3)[C@H](O)[C@@H](O)[C@@H]1O]

(24) TFC-039, represented by formula (24):

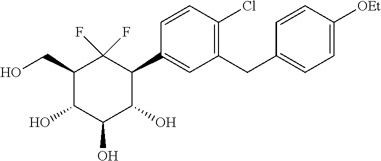 [SMILES: CCOc1ccc(Cc2cc([C@H]3[C@H](O)[C@@H](O)[C@H](O)[C@@H](CO)C3(F)F)ccc2Cl)cc1]

A corresponding method of preventing and/or treating one or more cardiac diseases in a non-human mammal/non-human mammal patient, in particular a canine/canine patient or a feline/feline patient, comprising administering the above exemplified one or more SGLT-2 inhibitors or pharmaceutically acceptable forms thereof in combination with pimobendan and/or telmisartan or pharmaceutically acceptable forms thereof to such non-human mammal/non-human mammal patient as well as the corresponding use of the above exemplified one or more SGLT-2 inhibitors or pharmaceutically acceptable forms thereof in combination with pimobendan and/or telmisartan or pharmaceutically acceptable forms thereof for the preparation of a medicament for the prevention and/or treatment of one or more cardiac diseases in a non-human mammal/non-human mammal patient are also intended to be comprised by the present invention.

In yet another aspect, the present invention also concerns the one or more SGLT-2 inhibitors or pharmaceutically acceptable forms thereof in combination with pimobendan and/or telmisartan or pharmaceutically acceptable forms thereof for use as herein disclosed and/or claimed, wherein the pharmaceutically acceptable form thereof is a crystalline complex between the one or more SGLT-2 inhibitors and one or more amino acids, preferably proline, more preferably L-proline; and most preferably is co-crystal of the one or more SGLT2 inhibitors, L-proline and crystalline water.

A corresponding method of preventing and/or treating one or more cardiac diseases in a non-human mammal/non-human mammal patient, in particular a canine/canine patient or a feline/feline patient, comprising administering one or more SGLT-2 inhibitors or pharmaceutically acceptable forms thereof in combination with pimobendan and/or telmisartan or pharmaceutically acceptable forms thereof to such non-human mammal/non-human mammal patient as well as the corresponding use of one or more SGLT-2 inhibitors or pharmaceutically acceptable forms thereof in combination with pimobendan and/or telmisartan or pharmaceutically acceptable forms thereof for the preparation of a medicament for the prevention and/or treatment of one or more cardiac diseases in a non-human mammal/non-human mammal patient are also intended to be comprised by the present invention.

In yet another aspect, the present invention also concerns the one or more SGLT-2 inhibitors or pharmaceutically acceptable forms thereof in combination with pimobendan and/or telmisartan or pharmaceutically acceptable forms thereof for use as herein disclosed and/or claimed, wherein velagliflozin or pharmaceutically acceptable forms thereof is to be administered as single SGLT-2 inhibitor in combination with pimobendan and/or telmisartan or pharmaceutically acceptable forms thereof: preferably wherein velagliflozin or pharmaceutically acceptable forms thereof is to be administered as single SGLT-2 inhibitor in combination with pimobendan; or wherein bexagliflozin or pharmaceutically acceptable forms thereof is to be administered as single SGLT-2 inhibitor in combination with pimobendan and/or telmisartan or pharmaceutically acceptable forms thereof: preferably wherein bexagliflozin or pharmaceutically acceptable forms thereof is to be administered as single SGLT-2 inhibitor in combination with pimobendan. In a preferred embodiment, velagliflozin or pharmaceutically acceptable forms thereof is combined with pimobendan only. In another embodiment, velagliflozin or pharmaceutically acceptable forms thereof is combined with pimobendan and telmisartan or pharmaceutically acceptable forms thereof. In another embodiment, velagliflozin or pharmaceutically acceptable forms thereof is combined with telmisartan or pharmaceutically acceptable forms thereof only. In another preferred embodiment, bexagliflozin or pharmaceutically acceptable forms thereof is combined with pimobendan only. In another embodiment, bexagliflozin or pharmaceutically acceptable forms thereof is combined with pimobendan and telmisartan or pharmaceutically acceptable forms thereof. In another embodiment, bexagliflozin or pharmaceutically acceptable forms thereof is combined with telmisartan or pharmaceutically acceptable forms thereof only.

Accordingly, the present invention also concerns the following further preferred embodiments:

Velagliflozin or pharmaceutically acceptable forms thereof as single SGLT-2 inhibitor in combination with pimobendan and/or telmisartan or pharmaceutically acceptable forms thereof for use in a method of prevention and/or treatment of DCM or MVD in a canine/canine patient.

Velagliflozin or pharmaceutically acceptable forms thereof as single SGLT-2 inhibitor in combination with pimobendan and/or telmisartan or pharmaceutically acceptable forms thereof for use in a method of prevention and/or treatment of HCM in a feline/feline patient.

Velagliflozin or pharmaceutically acceptable forms thereof as single SGLT-2 inhibitor in combination with pimobendan and/or telmisartan or pharmaceutically acceptable forms thereof for use in a method of treatment of DCM or MVD in a canine/canine patient.

Velagliflozin or pharmaceutically acceptable forms thereof as single SGLT-2 inhibitor in combination with pimobendan and/or telmisartan or pharmaceutically acceptable forms thereof for use in a method of treatment of HCM in a feline/feline patient.

Velagliflozin or pharmaceutically acceptable forms thereof as single SGLT-2 inhibitor in combination with pimobendan for use in a method of treatment of DCM or MVD in a canine/canine patient.

Velagliflozin or pharmaceutically acceptable forms thereof as as single SGLT-2 inhibitor in combination with pimobendan for use in a method of treatment of HCM in a feline/feline patient.

The present invention also concerns the following particularly preferred embodiments:

Velagliflozin or pharmaceutically acceptable forms thereof as single SGLT-2 inhibitor in combination with pimobendan and/or telmisartan or pharmaceutically acceptable forms thereof for use in a method of prevention and/or treatment of DCM or MVD in a canine/canine patient.

Velagliflozin or pharmaceutically acceptable forms thereof as single SGLT-2 inhibitor in combination with pimobendan and/or telmisartan or pharmaceutically acceptable forms thereof for use in a method of prevention and/or treatment of HCM in a feline/feline patient.

Velagliflozin or pharmaceutically acceptable forms thereof as single SGLT-2 inhibitor in combination with pimobendan and/or telmisartan or pharmaceutically acceptable forms thereof for use in a method of treatment of DCM or MVD in a canine/canine patient.

Velagliflozin or pharmaceutically acceptable forms thereof as single SGLT-2 inhibitor in combination with pimobendan and/or telmisartan or pharmaceutically acceptable forms thereof for use in a method of treatment of HCM in a feline/feline patient.

Velagliflozin or pharmaceutically acceptable forms thereof as single SGLT-2 inhibitor in combination with pimobendan for use in a method of treatment of DCM or MVD in a canine/canine patient.

Velagliflozin or pharmaceutically acceptable forms thereof as single SGLT-2 inhibitor in combination with pimobendan for use in a method of treatment of HCM in a feline/feline patient.

A corresponding method of preventing and/or treating one or more cardiac diseases in a non-human mammal/non-human mammal patient, in particular a canine/canine patient or a feline/feline patient, comprising administering one or more SGLT-2 inhibitors or pharmaceutically acceptable forms thereof in combination with pimobendan and/or telmisartan or pharmaceutically acceptable forms thereof to such non-human mammal/non-human mammal patient as well as the corresponding use of one or more SGLT-2 inhibitors or pharmaceutically acceptable forms thereof in combination with pimobendan and/or telmisartan or pharmaceutically acceptable forms thereof for the preparation of a medicament for the prevention and/or treatment of one or more cardiac diseases in a non-human mammal/non-human mammal patient are also intended to be comprised by the present invention.

In yet another aspect, the present invention also concerns the one or more SGLT-2 inhibitors or pharmaceutically acceptable forms thereof in combination with pimobendan and/or telmisartan or pharmaceutically acceptable forms thereof for use as herein disclosed and/or claimed, wherein the one or more SGLT-2 inhibitors or pharmaceutically acceptable forms thereof are administered orally, parenterally, intravenously, subcutaneously or intramuscularly, preferably orally.

A corresponding method of preventing and/or treating one or more cardiac diseases in a non-human mammal/non-human mammal patient, in particular a canine/canine patient or a feline/feline patient, comprising administering one or more SGLT-2 inhibitors or pharmaceutically acceptable forms thereof in combination with pimobendan and/or telmisartan or pharmaceutically acceptable forms thereof to such non-human mammal/non-human mammal patient as well as the corresponding use of one or more SGLT-2 inhibitors or pharmaceutically acceptable forms thereof in combination with pimobendan and/or telmisartan or pharmaceutically acceptable forms thereof for the preparation of a medicament for the prevention and/or treatment of one or more cardiac diseases in a non-human mammal/non-human mammal patient are also intended to be comprised by the present invention.

In yet another aspect, the present invention also concerns the one or more SGLT-2 inhibitors or pharmaceutically acceptable forms thereof in combination with pimobendan and/or telmisartan or pharmaceutically acceptable forms thereof for use as herein disclosed and/or claimed, wherein the one or more SGLT-2 inhibitors or pharmaceutically acceptable forms thereof are to be administered at a dose of 0.01 mg/kg bodyweight to 10 mg/kg bodyweight per day, preferably at a dose of 0.01 mg/kg bodyweight to 5 mg/kg bodyweight per day, more preferably at a dose of 0.01 mg/kg bodyweight to 4 mg/kg bodyweight per day, even more preferably at a dose of 0.01 mg/kg bodyweight to 3 mg/kg bodyweight per day, even more preferably at a dose of 0.01 mg/kg bodyweight to 2 mg/kg bodyweight per day, even more preferably at a dose of 0.01 mg/kg body-weight to 1 mg/kg bodyweight per day, even more preferably at a dose of 0.01 mg/kg bodyweight to 0.5 mg/kg bodyweight per day, most preferably at a dose of 0.01 mg/kg bodyweight to 0.3 mg/kg bodyweight per day. Alternatively, the one or more SGLT-2 inhibitors or pharmaceutically acceptable forms thereof are to be administered at a dose of 0.1 mg/kg bodyweight to 10 mg/kg bodyweight per day, preferably at a dose of 0.1 mg/kg bodyweight to 5 mg/kg bodyweight per day, more preferably at a dose of 0.1 mg/kg bodyweight to 4 mg/kg bodyweight per day, even more preferably at a dose of 0.1 mg/kg bodyweight to 3 mg/kg body-weight per day, even more preferably at a dose of 0.1 mg/kg bodyweight to 2 mg/kg bodyweight per day, even more preferably at a dose of 0.1 mg/kg bodyweight to 1 mg/kg bodyweight per day, even more preferably at a dose of 0.1 mg/kg bodyweight to 0.5 mg/kg bodyweight per day, most preferably at a dose of 0.1 mg/kg bodyweight to 0.3 mg/kg bodyweight per day.

A corresponding method of preventing and/or treating one or more cardiac diseases in a non-human mammal/non-human mammal patient, in particular a canine/canine patient or a feline/feline patient, comprising administering one or more SGLT-2 inhibitors or pharmaceutically acceptable forms thereof in combination with pimobendan and/or telmisartan or pharmaceutically acceptable forms thereof to such non-human mammal/non-human mammal patient as well as the corresponding use of one or more SGLT-2 inhibitors or pharmaceutically acceptable forms thereof in combination with pimobendan and/or telmisartan or pharmaceutically acceptable forms thereof for the preparation of a medicament for the prevention and/or treatment of one or more cardiac diseases in a non-human mammal/non-human mammal patient are also intended to be comprised by the present invention.

In yet another aspect, the present invention also concerns the one or more SGLT-2 inhibitors or pharmaceutically acceptable forms thereof in combination with pimobendan and/or telmisartan or pharmaceutically acceptable forms thereof for use as herein disclosed and/or claimed, wherein such one or more SGLT-2 inhibitors or pharmaceutically acceptable forms thereof is to be administered once per day or twice per day, preferably once per day.

A corresponding method of preventing and/or treating one or more cardiac diseases in a non-human mammal/non-human mammal patient, in particular a canine/canine patient or a feline/feline patient, comprising administering one or more SGLT-2 inhibitors or pharmaceutically acceptable forms thereof in combination with pimobendan and/or telmisartan or pharmaceutically acceptable forms thereof to such non-human mammal/non-human mammal patient as well as the corresponding use of one or more SGLT-2 inhibitors or pharmaceutically acceptable forms thereof in combination with pimobendan and/or telmisartan or pharmaceutically acceptable forms thereof for the preparation of a medicament for the prevention and/or treatment of one or more cardiac diseases in a non-human mammal/non-human mammal patient are also intended to be comprised by the present invention.

In yet another aspect, the present invention also concerns the one or more SGLT-2 inhibitors or pharmaceutically acceptable forms thereof in combination with pimobendan and/or telmisartan or pharmaceutically acceptable forms thereof for use as herein disclosed and/or claimed, wherein pimobendan is to be administered at a dose of 0.1 mg/kg bodyweight to 1 mg/kg bodyweight per day, preferably 0.2 mg/kg bodyweight to 0.6 mg/kg bodyweight per day, more preferably 0.5 mg/kg bodyweight per day.

A corresponding method of preventing and/or treating one or more cardiac diseases in a non-human mammal/non-human mammal patient, in particular a canine/canine patient or a feline/feline patient, comprising administering one or more SGLT-2 inhibitors or pharmaceutically acceptable forms thereof in combination with pimobendan and/or telmisartan or pharmaceutically acceptable forms thereof to such non-human mammal/non-human mammal patient as well as the corresponding use of one or more SGLT-2 inhibitors or pharmaceutically acceptable forms thereof in combination with pimobendan and/or telmisartan or pharmaceutically acceptable forms thereof for the preparation of a medicament for the prevention and/or treatment of one or more cardiac diseases in a non-human mammal/non-human mammal patient are also intended to be comprised by the present invention.

In yet another aspect, the present invention also concerns the one or more SGLT-2 inhibitors or pharmaceutically acceptable forms thereof in combination with pimobendan and/or telmisartan or pharmaceutically acceptable forms thereof for use as herein disclosed and/or claimed, wherein pimobendan is to be administered once per day or twice per day, preferably twice a day, more preferably every twelve hours.

A corresponding method of preventing and/or treating one or more cardiac diseases in a non-human mammal/non-human mammal patient, in particular a canine/canine patient or a feline/feline patient, comprising administering one or more SGLT-2 inhibitors or pharmaceutically acceptable forms thereof in combination with pimobendan and/or telmisartan or pharmaceutically acceptable forms thereof to such non-human mammal/non-human mammal patient as well as the corresponding use of one or more SGLT-2 inhibitors or pharmaceutically acceptable forms thereof in combination with pimobendan and/or telmisartan or pharmaceutically acceptable forms thereof for the preparation of a medicament for the prevention and/or treatment of one or more cardiac diseases in a non-human mammal/non-human mammal patient are also intended to be comprised by the present invention.

In yet another aspect, the present invention also concerns the one or more SGLT-2 inhibitors or pharmaceutically acceptable forms thereof in combination with pimobendan and/or telmisartan or pharmaceutically acceptable forms thereof for use as herein disclosed and/or claimed, wherein the one or more SGLT-2 inhibitors are administered in combination with pimobendan and telmisartan or pharmaceutically acceptable forms thereof, preferably wherein the one or more SGLT-2 inhibitors or pharmaceutically acceptable forms thereof are to be administered before, after or concomitantly with administering pimobendan and telmisartan or pharmaceutically acceptable forms thereof.

A corresponding method of preventing and/or treating one or more cardiac diseases in a non-human mammal/non-human mammal patient, in particular a canine/canine patient or a feline/feline patient, comprising administering one or more SGLT-2 inhibitors or pharmaceutically acceptable forms thereof in combination with pimobendan and/or telmisartan or pharmaceutically acceptable forms thereof to such non-human mammal/non-human mammal patient as well as the corresponding use of one or more SGLT-2 inhibitors or pharmaceutically acceptable forms thereof in combination with pimobendan and/or telmisartan or pharmaceutically acceptable forms thereof for the preparation of a medicament for the prevention and/or treatment of one or more cardiac diseases in a non-human mammal/non-human mammal patient are also intended to be comprised by the present invention.

In yet another aspect, the present invention also concerns the one or more SGLT-2 inhibitors or pharmaceutically acceptable forms thereof in combination with pimobendan and/or telmisartan or pharmaceutically acceptable forms thereof for use as herein disclosed and/or claimed, wherein telmisartan or pharmaceutically acceptable forms thereof is to be administered at a dose of 0.01 to 10 mg/kg of bodyweight per day, preferably 0.05 to 8 mg/kg of bodyweight per day, even more preferably 0.1 to 5 mg/kg of bodyweight per day, even more preferably 0.2 to 4 mg/kg of bodyweight per day, even more preferably 0.3 to 3 mg/kg of bodyweight per day, even more preferably 0.4 to 2.5 mg/kg of bodyweight per day, even more preferably 0.5 to 2 mg/kg of bodyweight per day, most preferably 0.75 to 1.5 mg/kg of bodyweight per day.

A corresponding method of preventing and/or treating one or more cardiac diseases in a non-human mammal/non-human mammal patient, in particular a canine/canine patient or a feline/feline patient, comprising administering one or more SGLT-2 inhibitors or pharmaceutically acceptable forms thereof in combination with pimobendan and/or telmisartan or pharmaceutically acceptable forms thereof to such non-human mammal/non-human mammal patient as well as the corresponding use of one or more SGLT-2 inhibitors or pharmaceutically acceptable forms thereof in combination with pimobendan and/or telmisartan or pharmaceutically acceptable forms thereof for the preparation of a medicament for the prevention and/or treatment of one or more cardiac diseases in a non-human mammal/non-human mammal patient are also intended to be comprised by the present invention.

In yet another aspect, the present invention also concerns the one or more SGLT-2 inhibitors or pharmaceutically acceptable forms thereof in combination with pimobendan and/or telmisartan or pharmaceutically acceptable forms thereof for use as herein disclosed and/or claimed, wherein telmisartan or pharmaceutically acceptable forms thereof is to be administered once per day or twice per day, preferably once a day.

A corresponding method of preventing and/or treating one or more cardiac diseases in a non-human mammal/non-human mammal patient, in particular a canine/canine patient or a feline/feline patient, comprising administering one or more SGLT-2 inhibitors or pharmaceutically acceptable forms thereof in combination with pimobendan and/or telmisartan or pharmaceutically acceptable forms thereof to such non-human mammal/non-human mammal patient as well as the corresponding use of one or more SGLT-2 inhibitors or pharmaceutically acceptable forms thereof in combination with pimobendan and/or telmisartan or pharmaceutically acceptable forms thereof for the preparation of a medicament for the prevention and/or treatment of one or more cardiac diseases in a non-human mammal/non-human mammal patient are also intended to be comprised by the present invention.

In yet another aspect, the present invention also concerns the one or more SGLT-2 inhibitors or pharmaceutically acceptable forms thereof in combination with pimobendan and/or telmisartan or pharmaceutically acceptable forms thereof for use as herein disclosed and/or claimed, wherein the preventive and/or therapeutic effect is characterized by one or more of the following clinical and/or biochemical parameters:
improved cardiometabolic efficiency, characterized by an increased ratio of [cardiac output/metabolic substrate consumed] and/or characterized by an increased ratio of [cardiac output/oxygen consumed];
increase of the production of ketone bodies in the liver, characterized by increased plasma levels of 3-hydroxybutyric acid and/or the corresponding acylcamitines, i.e., hydroxy butyry lcarnitine, and increased plasma levels of one or more of the branched-chain amino acids (valine, leucine and isoleucine);
improved cardiac function by achieved reduced pre-and/or afterload, improved arterial wall structure function;
improved echocardiographic parameters, such as decreased LA (Left atrium dimension measured as right parasternal short-axis), LA/Ao (left atrium to aorta ratio; Ao=Aortic root diameter), IVSd (interventricular septal end diastolic dimension, i.e. the thickness of the interventricular septum), and/or LAD (Left atrium measured as right parasternal long-axis), and improved cardiac biomarkers, such as decreased NT-proBNP (N-terminal prohormone of brain natriuretic peptide) and/or decreased cTnI (cardiac Troponin I) and/or increased erythropoietin concentration and/or changes of metabolites indicating a change of cardiac energy sources; as well as improved heart murmur;
delayed onset of different phenotypes of cardiac diseases, such as (M)MVD and/or DCM and/or cardiomyopathies, preferably at least by 1, 2, 3, 4, 5, 6, 7, 8, 9, 10, 11, 12, 13, 14, 15 or more months, or even stopped progression of different phenotypes of cardiac diseases, such as (M)MVD and/or DCM and/or cardiomyopathies;
longer time of survival, preferably at least by 1, 2, 3, 4, 5, 6, 7, 8, 9, 10, 11, 12, 13, 14, 15 or more months, and/or delay of next episode of heart failure, preferably at least by 1, 2, 3, 4, 5, 6, 7, 8, 9, 10, 11, 12, 13, 14, 15 or more months, and/or lower level of cardiac mortality and/or morbidity;
improved clinical signs, such as reduced e.g., breathlessness or dyspnea, cough, depression, exercise intolerance, inappetence, syncope, abdominal distention and/or polydipsia;
prolongation of time to event (e.g., heart failure, cardiac death, onset of clinical signs, need for additional concomitant medication, increase in dose of concomitant therapy-diuretics); prevention of fibrosis;
prevention of cardiomyocytes death/protection of cardiomyocytes (reduction of oxidative stress);
increase of vascular wall flexibility;
prevention of hypertension;
higher quality of life.

A corresponding method of preventing and/or treating one or more cardiac diseases in a non-human mammal/non-human mammal patient, in particular a canine/canine patient or a feline/feline patient, comprising administering one or more SGLT-2 inhibitors or pharmaceutically acceptable forms thereof in combination with pimobendan and/or telmisartan or pharmaceutically acceptable forms thereof to such non-human mammal/non-human mammal patient as well as the corresponding use of one or more SGLT-2 inhibitors or pharmaceutically acceptable forms thereof in combination with pimobendan and/or telmisartan or pharmaceutically acceptable forms thereof for the preparation of a medicament for the prevention and/or treatment of one or more cardiac diseases in a non-human mammal/non-human mammal patient are also intended to be comprised by the present invention.

In yet another aspect, the present invention also concerns a pharmaceutical composition comprising one or more SGLT2 inhibitors or pharmaceutically acceptable forms thereof in combination with pimobendan and/or telmisartan or pharmaceutically acceptable forms thereof as herein disclosed and/or claimed for use herein disclosed and/or claimed, wherein preferably such pharmaceutical composition is a fixed-dose-combination (FDC) of the one or more SGLT-2 inhibitors or pharmaceutically acceptable forms thereof and pimobendan and/or telmisartan or pharmaceutically acceptable forms thereof, wherein more preferably such FDC is a solid or a liquid formulation.

In yet another aspect, the present invention also concerns the one or more SGLT-2 inhibitors or pharmaceutically acceptable forms thereof as herein disclosed and/or claimed for use as a diuretic, optionally in combination with pimobendan and/or telmisartan or pharmaceutically acceptable forms thereof, in particular in the prevention and/or treatment of congestion, preferably fluid congestion (e.g., in heart failure, acute heart failure, congestive heart failure, oedema, pulmonary oedema, pulmonary congestion, ascites, decrease of the workload on the heart and arteries); and/or reduction of fluid volume excess, such as accumulation of fluid in body cavities, acute tissue oedema; and/or asthma; and/or forced diuresis (e.g., oliguria, intoxinations) in a non-human mammal.

In one preferred embodiment, velagliflozin or pharmaceutically acceptable forms thereof is used as a diuretic, optionally in combination with pimobendan and/or telmisartan or pharmaceutically acceptable forms thereof, in particular in the prevention and/or treatment of congestion, preferably fluid congestion (e.g., in heart failure, acute heart failure, congestive heart failure, oedema, pulmonary oedema, pulmonary congestion, ascites, decrease of the workload on the heart and arteries); and/or reduction of fluid volume excess, such as accumulation of fluid in body cavities, acute tissue oedema: and/or asthma: and/or support forced diuresis (e.g., oliguria, intoxinations). In another preferred embodiment, bexagliflozin or pharmaceutically acceptable forms thereof is used as a diuretic, optionally in combination with pimobendan and/or telmisartan or pharmaceutically acceptable forms thereof, in particular in the prevention and/or treatment of congestion, preferably fluid congestion (e.g., in heart failure, acute heart failure, congestive heart failure, oedema, pulmonary oedema, pulmonary congestion, ascites, decrease of the workload on the heart and arteries); and/or reduction of fluid volume excess, such as accumulation of fluid in body cavities, acute tissue oedema, and/or asthma; and/or support forced diuresis (e.g., oliguria, intoxinations).

A corresponding method of using the one or more SGLT-2 inhibitors or pharmaceutically acceptable forms thereof as herein disclosed and/or claimed as a diuretic in a non-human mammal/non-human mammal patient, in particular in the prevention and/or treatment of congestion, preferably fluid congestion (e.g., in heart failure, acute heart failure, congestive heart failure, oedema, pulmonary oedema, pulmonary congestion, ascites, decrease of the workload on the heart and arteries); and/or reduction of fluid volume excess, such as accumulation of fluid in body cavities, acute tissue oedema; and/or asthma; and/or support forced diuresis (e.g., oliguria, intoxinations) in a non-human mammal/non-human mammal patient, in particular a canine/canine patient or a feline/feline patient, comprising administering one or more SGLT-2 inhibitors or pharmaceutically acceptable forms thereof, optionally in combination with pimobendan and/or telmisartan or pharmaceutically acceptable forms thereof, to such non-human mammal/non-human mammal patient as well as the corresponding use of one or more SGLT-2 inhibitors or pharmaceutically acceptable forms thereof, optionally in combination with pimobendan and/or telmisartan or pharmaceutically acceptable forms thereof, as a diuretic in a non-human mammal/non-human mammal patient, in particular for the preparation of a medicament for in the prevention and/or treatment of congestion, preferably fluid congestion (e.g., in heart failure, acute heart failure, congestive heart failure, oedema, pulmonary oedema, pulmonary congestion, ascites, decrease of the workload on the heart and arteries); and/or reduction of fluid volume excess, such as accumulation of fluid in body cavities, acute tissue oedema: and/or asthma; and/or support forced diuresis (e.g., oliguria, intoxinations) in a non-human mammal/non-human mammal patient are also intended to be comprised by the present invention.

The advantages according to the present invention are one or more of the following:
dose reduction of the individual active ingredients (independently from each other) and/or replacement of further concomitant treatments (e.g. diuretics) as compared to single respective treatment
dose reduction of further concomitant treatments (e.g. diuretics) as compared to standard of care treatment
higher cardiac efficiency
increased heart health via cardiomyocyte protection
less side effects (e.g. cardiac death, hospitalization)

### DETAILED DESCRIPTION OF THE INVENTION

Before the embodiments of the present invention are described in further detail, it shall be noted that as used herein and in the appended claims, the singular forms “a”, “an”, and “the” include plural reference unless the context clearly dictates otherwise.

Unless defined otherwise, all technical and scientific terms used herein have the same meanings as commonly understood by one of ordinary skill in the art to which this invention belongs. All given ranges and values may vary by 1 to 5% unless indicated otherwise or known otherwise by the person skilled in the art, therefore, the term “about” was usually omitted from the description and claims. Although any methods and materials similar or equivalent to those described herein can be used in the practice or testing of the present invention, the preferred methods, devices, and materials are now described. All publications mentioned herein are incorporated herein by reference for the purpose of describing and disclosing the substances, excipients, carriers, and methodologies as reported in the publications which might be used in connection with the invention. Nothing herein is to be construed as an admission that the invention is not entitled to antedate such disclosure by virtue of prior invention.

In a preferred embodiment, the “non-human mammal” is selected from the group consisting of: bovine, canine, caprine, equine, feline, lagomorphs, ovine, porcine, rodent; more preferably is selected from the group consisting of: cattle, cow, dog, cat, goat, horse, pony, donkey, sheep, pig, rabbit, rat, mouse; even more preferably selected from the group consisting of: canine or feline; most preferably selected from the group consisting of: dog or cat.

In the course of the present invention, the term “canine animal” or “canine” refers to any member of the canidae family (i.e., a canid). It may thus belong either to the subfamily canidae or the subfamily caninae. The term canine animal encompasses the term dog, e.g., a domestic dog. The term domestic dog encompasses the terms Canis familiaris or Canis lupus familiaris. Most preferably, the canine animal or canine is a dog, in particular a domestic dog.

In the course of the present invention, the term “feline animal” or “feline” refers to any member of the Felidae family (i.e., a felid). It may thus belong either to the subfamily felinae or the subfamily pantherinae. The term feline animal encompasses the term cat, e.g., a domestic cat. The term domestic cat encompasses the terms Felis catus and Felis silvestris catus. Most preferably, the feline animal or feline is a cat, in particular a domestic cat.

In cats, the most commonly seen sign of heart failure is the development of difficult breathing (dyspnoea) and/or more rapid breathing (tachypnoea). This is generally caused by either a build-up of fluid in the chest cavity around the lungs (called a pleural effusion), or due to a build-up of fluid within the lungs themselves (called pulmonary oedema). Along with breathing difficulties, cats may have cold extremities (e.g., ears and paws), and may have pale mucous membranes (gums and eyes) suggesting poor circulation. Occasionally, the mucous membranes of the mouth and eyes, and even the skin, may show signs of cyanosis (a bluish colour). All these clinical signs of heart failure are improved in cats after treatment with SGLT-2 inhibitors or clinically relevant delayed in occurrence as compared to the untreated course of the disease development. Another sign which can occur in cats affected by heart diseases as cardiomyopathy and may sometimes be the first indicator of underlying heart disease, is the development of what is known as ‘feline aortic thromboembolism’ (FATE). A thrombus (blood clot) may develop within one of the heart chambers (usually left atrium) in a cat with cardiomyopathy. This occurs mainly because the blood is not flowing normally through the heart. The thrombus, or clot, is initially attached to the wall of the heart, but may become dislodged and be carried into the blood leaving the heart. A thrombus that moves into the blood circulation is called an embolus, hence the term ‘thromboembolism’. Once in the circulation, these emboli can lodge in small arteries and obstruct the flow of blood to regions of the body. Although this can happen at a number of different sites, it more commonly occurs towards the end of the major artery that leaves the heart (the aorta) as it divides to supply blood to the back legs. This complication is seen most commonly with HCM and will cause a sudden onset of paralysis to one or both back legs, with severe pain and considerable distress. Also, this clinical sign of heart failure is improved/delayed in cats after treatment with SGLT-2 inhibitors in combination with pimobendan and/or telmisartan or pharmaceutically acceptable forms thereof or clinically relevant delayed in occurrence as compared to the untreated course of the disease development, preferably at least by 1, 2, 3, 4, 5, 6, 7, 8, 9, 10, 11, 12, 13, 14, 15 or more months. Overall, the survival time of cats with cardiac disease(s) after treatment with SGLT-2 inhibitors in combination with pimobendan and/or telmisartan or pharmaceutically acceptable forms thereof is clinically relevant increased at least by 1, 2, 3, 4, 5, 6, 7, 8, 9, 10, 11, 12, 13, 14, 15 or more months, preferably at least by 6 months, as compared to untreated cats with cardiac disease(s).

In the course of the present invention, the term “heart disease” is synonymous with “cardiac disease” and refers to any disorder and deformities of the heart itself, which affect the heart's structure and function. There are many types of heart disease that affect different parts of the organ and occur in different ways including congenital heart diseases (e.g., septal defects, obstruction defects), arrhythmias (e.g., tachycardia, bradycardia and fibrillation) and cardiomyopathies.

In the course of the present invention, the term “heart failure”, also known as congestive heart failure and congestive cardiac failure, refers to the pathophysiological process in which the heart cannot pump sufficiently to maintain the blood flow through the body to meet the metabolic requirements (oxygen and substrates) of peripheral tissues and organs. It can also be defined as a complex clinical syndrome that is based on abnormal structure or function of the heart and which is characterized by symptoms like exercise intolerance, dyspnoea, fatigue, fluid retention and reduced longevity. It can be divided into systolic failure, where the ejection of blood out of the heart in the systole is affected, and diastolic failure, where the heart is not able to receive enough blood in the ventricular cavities at low pressure during diastole. It is mostly a chronic disease due to a chronic work overload of the heart or developed after an acute hemodynamic stress due to fluid overload, a valvular dysfunction or a myocardial infarction.

In the course of the present invention, the term “cardiomyopathy” refers to a group of diseases that affect the heart muscle being the most common form of heart disease seen in cats, and the most common cause of heart failure. Cardiomyopathies are described according to the effect they have on the structure and function of the cardiac muscle. Types of cardiomyopathy include: hypertrophic cardiomyopathy (HCM), hypertrophic obstructive cardiomyopathy (HOCM), restrictive cardiomyopathy (RCM), unclassified cardiomyopathy

(UCM), arrhythmogenic right ventricular cardiomyopathy (ARVC) and dilated cardiomyopathy (DCM). The classification is based on echocardiographic measurements.

Hypertrophic cardiomyopathy (HCM) is the most prevalent feline cardiac disorder. It affects most commonly middle-aged cats (average 6.5 years), but all ages are affected. There is a male predisposition (>75%). In humans, there is an important hereditary predisposition for HCM in 55% of cases. In people, this disorder may be congenital or acquired, and probably represents a group of diseases. Although the etiology of feline HCM is unknown, the Persian and Maine coon cat have appeared to be predisposed in some case series, suggesting a genetic influence. As is the case with systemic hypertension, hyperthyroidism, and aortic stenosis, HCM is associated with marked left ventricular hypertrophy, but in this instance, no underlying cause can be identified. Cardiac lesions are typified by severe left ventricular concentric hypertrophy and secondary left atrial dilatation. Asymmetric septal hypertrophy (ASH), present in the majority of dogs and humans with HCM, is present in only 30% of cats with HCM. Histological cardiac myofiber disarray is reported in 27% of affected cats and only in those with asymmetric septal hypertrophy. Other histological features of feline HCM include myocardial and endocardial fibrosis and narrowed coronary arteries. Dynamic aortic outflow obstruction, secondary mitral insufficiency, myocardial ischemia, and systemic arterial embolism (SAE) may complicate this syndrome. The left heart is predominately affected and clinical signs manifested as sudden death or, more commonly, acute left heart failure due to diastolic dysfunction. Pleural effusion is occasionally associated with HCM. Systolic function is usually adequate or enhanced. Stressful incidents, such as a car ride, restraint for an ECG, confrontation with a dog, or an embolic event may precipitate in left heart failure and pulmonary oedema.

A hypertrophic obstructive cardiomyopathy (HOCM) is characterized by a left ventricular hypertrophy combined with an outflow obstruction of the left ventricle into the aorta. The degree of obstruction and clinical presentation is dependent upon the extent of hypertrophy. It most commonly affects the ventricular septum, although any portion of the left ventricle can be affected.

Restrictive Cardiomyopathy (RCM) occurs when ventricular diastolic compliance is impaired (i.e., stiffness is increased) by infiltration of the endocardium, subendocardium, or myocardium by fibrous tissue or another component. In contrast to human medicine where specific causes, such as amyloidosis and eosinophilic infiltration are causes of RCM, specific causes for RCM have not been clearly defined in the cat. Without the use of invasive diagnostic procedures to directly measure left ventricular diastolic function, DTI, other indirect measures of diastolic function, or necropsy examination, it is often impossible to distinguish this disorder from the form or forms of unclassified cardiomyopathy that are idiopathic. The precise etiology of feline RCM is unknown. However, there is some evidence that it may be inflammatory in nature.

Dilated cardiomyopathy (DCM) is characterized by dilated or enlarged heart chambers and reduced contraction ability. Before 1987, DCM was one of the most common heart diseases in cats. This is suspected to have been related to a dietary deficiency of the amino acid taurine. Today DCM in cats is relatively rare, since most cat food manufacturers began adding taurine supplements to their foods, further confirming the relationship. Some breeds, such as the Burmese, Abyssinian, and Siamese, are more commonly affected by DCM, but the underlying cause in the majority of cases remains unknown. The disease will usually affect cats between the ages of 2 to 20 years, but the average age of onset is ten years old.

Unclassified Cardiomyopathy (UCM): in recent years an increasing number of cats have been identified that do not fit into any recognized disease classification using echocardiographic and pathological criteria. Typically, these cats have severe biatrial enlargement, normal left ventricles or mild hypertrophy and normal or slightly decreased systolic function, but they do not have the typical post-mortem findings of fibrosis seen in restrictive cardiomyopathy. Many cats have enlargement of the right ventricle. It is not known if these cats represent a progressive or regressive form of other known cardiomyopathie states.

Arrhythmogenic Right Ventricular Cardiomyopathy (ARVC): this form of cardiomyopathy has recently been described in cats. The etiology is unknown, however a familial form has been reported in humans. It is characterized by severe right atrial and ventricular enlargement and marked tricuspid regurgitation due to distortion of the tricuspid valve: arrhythmias are common. It is possible that cases of ARVC have previously been misdiagnosed as tricuspid valve dysplasia.

In the course of the present invention, the term “(myxomatous) mitral valve disease” [(M)MVD] refers to the most common cardiovascular condition in dogs and the most frequent cause of (congestive) heart failure, affecting primally small breed dogs over 5 years of age. The pathophysiology of (myxomatous) mitral valve disease is characterized by the progressive dilation of the left ventricle and the left atrium resulting from degenerative changes and an insufficiency of the mitral valve. The valve defect leads to a blood back-flow and reduced ejection fraction and represents an additional effort to the heart causing an enlargement of the left ventricle that if untreated, weakens, leading to congestive heart failure (CHF).

In the course of the present invention, the term “dilated cardiomyopathy” (DCM) in connection with dogs refers to the second most common cardiovascular condition in dogs, affecting primarily larger dogs of all ages with an overall incidence of 8%. DCM is the disease of the cardiomyocytes, presenting itself by an enlargement of the left ventricle and left atrium or an enlargement of all the heart chambers and often the muscular walls of the heart are much thinner than normal. As a result, the ventricle's pump capacity becomes reduced and blood flow is impaired, leading to a congestion in the blood flow. As the disease progresses, it causes congestive heart failure (CHF).

In the course of the present invention, the term “asymptomatic (occult, preclinical) (myxomatous) mitral valve disease [(M)MVD]” relates to any contractile disorder or disease of the heart which is due to/secondary to (M)MVD-however, yet without any clinical symptoms of (congestive) heart failure. In particular, it relates to heart failure due to (M)MVD of ISACHC Class I (Class IA and/or Class IB), NYHA Class I and ACVIM stage B2.

In the course of the present invention, the term “asymptomatic (occult, preclinical) dilated cardiomyopathy (DCM)” relates to any contractile disorder or disease of the heart which is due to/secondary to DCM-however, yet without any clinical symptoms of (congestive) heart failure. In particular, it relates to heart failure due to DCM of ISACHC Class I.

In the course of the present invention, the term “one or more SGLT-2 inhibitors or pharmaceutically acceptable forms thereof in combination with pimobendan” relates to the medical combination of the two or more active ingredients, i.e. the one or more SGLT-2 inhibitors or pharmaceutically acceptable forms thereof are administered to the non-human mammal (patient) before, after or concomitantly with administering pimobendan. In this context, the two or more active ingredients can be present in the same dosage form and as a result are concomitantly administered. Alternatively, the two or more active ingredients can be present in separate (identical or different) dosage forms for administration after one another at different time points (“before” or “after” aspects) or at the same time point (“concomitantly” aspect).

SGLT-2 inhibitors for use according to the invention include, but are not limited to, glucopyranosyl-substituted benzene derivatives, for example as described in WO 01/27128, WO 03/099836, WO 2005/092877, WO 2006/034489, WO 2006/064033, WO 2006/117359, WO 2006/117360, WO 2007/025943, WO 2007/028814, WO 2007/031548, WO 2007/093610, WO 2007/128749, WO 2008/049923, WO 2008/055870, WO 2008/055940, WO 2009/022020 or WO 2009/022008.

Moreover, the one or more SGLT-2 inhibitors for use according to the invention may be selected from the group consisting of the following compounds or pharmaceutically acceptable forms thereof:
(1) a glucopyranosyl-substituted benzene derivative of the formula (1)

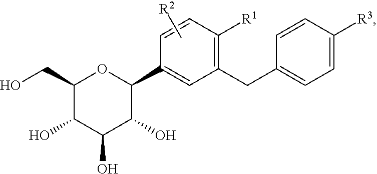

wherein R¹ denotes cyano, Cl or methyl (most preferably cyano);
R² denotes H, methyl, methoxy or hydroxy (most preferably H) and
R³ denotes cyclopropyl, hydrogen, fluorine, chlorine, bromine, iodine, methyl, ethyl, propyl, isopropyl, butyl, sec-butyl, iso-butyl, tert-butyl, 3-methyl-but-1-yl, cyclobutyl, cyclopentyl, cyclohexyl, 1-hydroxy-cyclopropyl, 1-hydroxy-cyclobutyl, 1-hydroxy-cyclopentyl, 1-hydroxy-cyclohexyl, ethinyl, ethoxy, difluoromethyl, trifluoromethyl, pentafluoroethyl, 2-hydroxyl-ethyl, hydroxymethyl, 3-hydroxy-propyl, 2-hydroxy-2-methyl-prop-1-yl, 3-hydroxy-3-methyl-but-1-yl, 1-hydroxy-1-methyl-ethyl, 2,2,2-trifluoro-1-hydroxy-1-methyl-ethyl, 2,2,2-trifluoro-1-hydroxy-1-trifluoromethyl-ethyl, 2-methoxy-ethyl, 2-ethoxy-ethyl, hydroxy, difluoromethyloxy, trifluoromethyloxy, 2-methyloxy-ethyloxy, methylsulfanyl, methylsulfinyl, methlysulfonyl, ethylsulfinyl, ethylsulfonyl, trimethylsilyl, (R)-tetrahydrofuran-3-yloxy or (S)-tetrahydrofuran-3-yloxy or cyano;
wherein R³ is preferably selected from cyclopropyl, ethyl, ethinyl, ethoxy, (R)-tetrahydrofuran-3-yloxy or (S)-tetrahydrofuran-3-yloxy; and most preferably R³ is cyclopropyl,
or a derivative thereof wherein one or more hydroxyl groups of the β-D-glucopyranosyl group are acylated with groups selected from (C₁₋₁₈-alkyl)carbonyl, (C₁₋₁₈-alkyl)oxycarbonyl, phenyl-carbonyl and phenyl-(C₁₋₃-alkyl)-carbonyl;
(2) Velagliflozin, represented by formula (2):

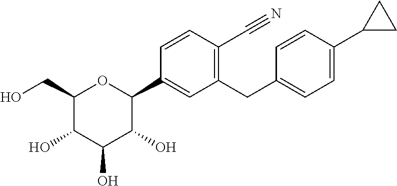 [SMILES: N#Cc1ccc([C@@H]2O[C@H](CO)[C@@H](O)[C@H](O)[C@H]2O)cc1Cc1ccc(C2CC2)cc1]

(3) Dapagliflozin, represented by formula (3):

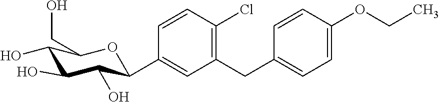

(4) Canagliflozin, represented by formula (4):

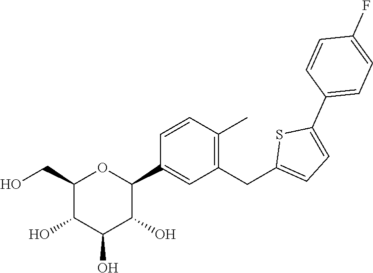 [SMILES: Cc1ccc([C@@H]2O[C@H](CO)[C@@H](O)[C@H](O)[C@H]2O)cc1Cc1ccc(-c2ccc(F)cc2)s1]

(5) Empagliflozin, represented by formula (5):

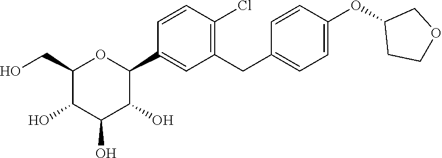 [SMILES: OC[C@H]1O[C@@H](c2ccc(Cl)c(Cc3ccc(O[C@H]4CCOC4)cc3)c2)[C@H](O)[C@@H](O)[C@@H]1O]

(6) Luseogliflozin, represented by formula (6):

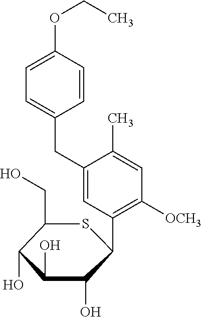

(7) Tofogliflozin, represented by formula (7):

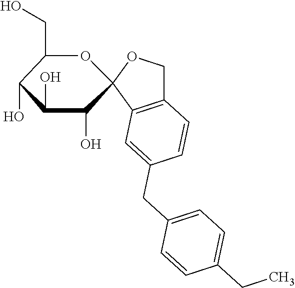

(8) Ipragliflozin, represented by formula (8):

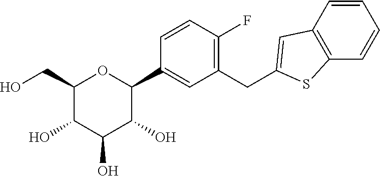 [SMILES: OC[C@H]1O[C@@H](c2ccc(F)c(Cc3cc4ccccc4s3)c2)[C@H](O)[C@@H](O)[C@@H]1O]

(9) Ertugliflozin, represented by formula (9):

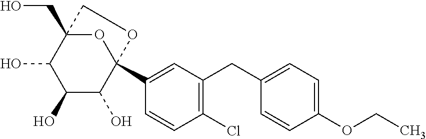 [SMILES: CCOc1ccc(Cc2cc([C@]34OC[C@](CO)(O3)[C@@H](O)[C@H](O)[C@H]4O)ccc2Cl)cc1]

(10) Atigliflozin, represented by formula (10):

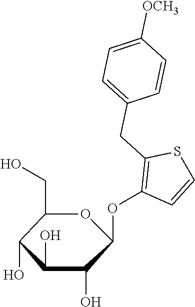

(11) Remogliflozin, represented by formula (11):

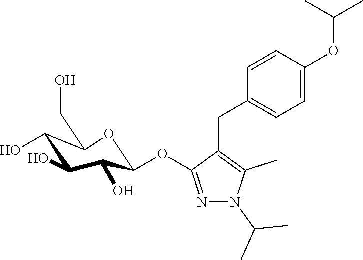

(11A) Remogliflozin etabonate, represented by formula (11A):

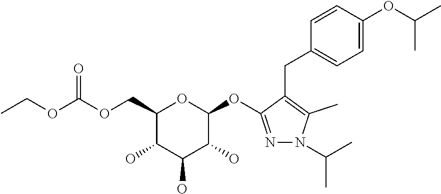 [SMILES: CCOC(=O)OC[C@H]1O[C@@H](Oc2nn(C(C)C)c(C)c2Cc2ccc(OC(C)C)cc2)[C@H](O)[C@@H](O)[C@@H]1O]

(12) a thiophene derivative of the formula (12)

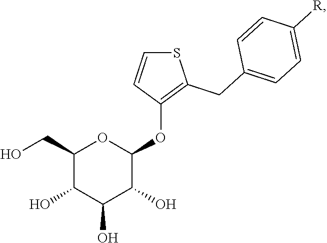

wherein R denotes methoxy or trifluoromethoxy;
(13) 1-(β-D-glucopyranosyl)-4-methyl-3-[5-(4-fluorophenyl)-2-thienylmethyl]benzene, represented by formula (13);

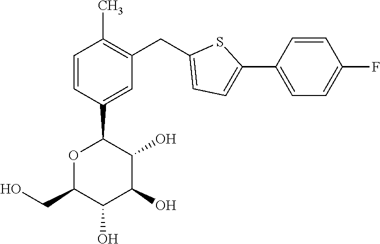 [SMILES: Cc1ccc([C@@H]2O[C@H](CO)[C@@H](O)[C@H](O)[C@H]2O)cc1Cc1ccc(-c2ccc(F)cc2)s1]

(14) a spiroketal derivative of the formula (14):

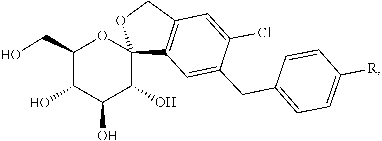

wherein R denotes methoxy, trifluoromethoxy, ethoxy, ethyl, isopropyl or tert. butyl;
(15) a pyrazole-O-glucoside derivative of the formula (15)

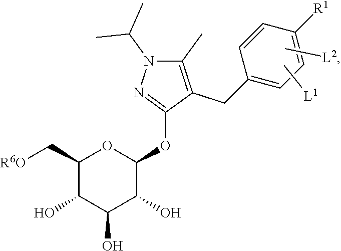

wherein
R¹ denotes C₁₋₃-alkoxy,
L¹, L² independently of each other denote H or F,
R⁶ denotes H, (C₁₋₃-alkyl)carbonyl, (C₁₋₆-alkyl)oxycarbonyl, phenyloxycarbonyl, benzyloxycarbonyl or benzylcarbonyl;
(16) Sotagliflozin, represented by formula (16):

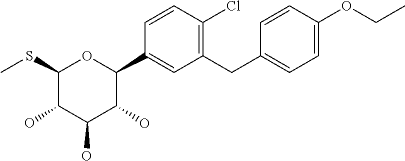 [SMILES: CCOc1ccc(Cc2cc([C@@H]3O[C@H](SC)[C@@H](O)[C@H](O)[C@H]3O)ccc2Cl)cc1]

(17) Sergliflozin, represented by formula (17):

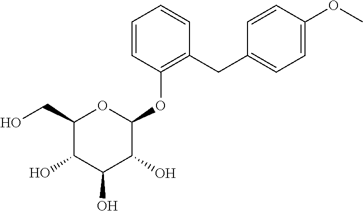 [SMILES: COc1ccc(Cc2ccccc2O[C@@H]2O[C@H](CO)[C@@H](O)[C@H](O)[C@H]2O)cc1]

(18) a compound represented by formula (18):

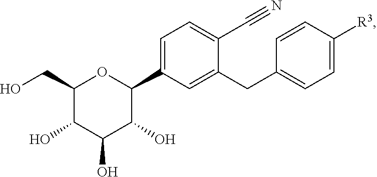

wherein
R³ denotes cyclopropyl, hydrogen, fluorine, chlorine, bromine, iodine, methyl, ethyl, propyl, isopropyl, butyl, sec-butyl, iso-butyl, tert-butyl, 3-methyl-but-1-yl, cyclobutyl, cyclopentyl, cyclohexyl, 1-hydroxy-cyclopropyl, 1-hydroxy-cyclobutyl, 1-hydroxy-cyclopentyl, 1-hydroxy-cyclohexyl, ethinyl, ethoxy, difluoromethyl, trifluoromethyl, pentafluoroethyl, 2-hydroxyl-ethyl, hydroxymethyl, 3-hydroxy-propyl, 2-hydroxy-2-methyl-prop-1-yl, 3-hydroxy-3-methyl-but-1-yl, 1-hydroxy-1-methyl-ethyl, 2,2,2-trifluoro-1-hydroxy-1-methyl-ethyl, 2,2,2-trifluoro-1-hydroxy-1-trifluoromethyl-ethyl, 2-methoxy-ethyl, 2-ethoxy-ethyl, hydroxy, difluoromethyloxy, trifluoromethyloxy, 2-methyloxy-ethyloxy, methylsulfanyl, methylsulfinyl, methlysulfonyl, ethylsulfinyl, ethylsulfonyl, trimethylsilyl, (R)-tetrahydrofuran-3-yloxy or (S)-tetrahydrofuran-3-yloxy or cyano, and wherein R³ is preferably selected from cyclopropyl, ethyl, ethinyl, ethoxy, (R)-tetrahydrofuran-3-yloxy or (S)-tetrahydrofuran-3-yloxy; and R³ most preferably is cyclopropyl,
or a derivative thereof wherein one or more hydroxyl groups of the β-D-glucopyranosyl group are acylated with groups selected from (C₁₋₁₈-alkyl)carbonyl, (C₁₋₁₈-alkyl)oxycarbonyl, phenylcarbonyl and phenyl-(C₁₋₃-alkyl)-carbonyl;
(19) Bexagliflozin, represented by formula (19):

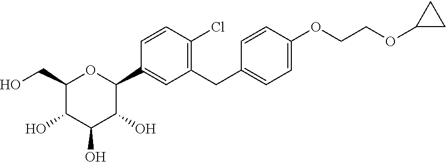 [SMILES: OC[C@H]1O[C@@H](c2ccc(Cl)c(Cc3ccc(OCCOC4CC4)cc3)c2)[C@H](O)[C@@H](O)[C@@H]1O]

(20) Janagliflozin, represented by formula (20):

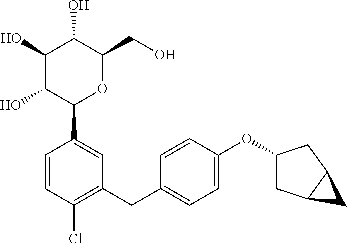 [SMILES: OC[C@H]1O[C@@H](c2ccc(Cl)c(Cc3ccc(O[C@H]4C[C@@H]5C[C@@H]5C4)cc3)c2)[C@H](O)[C@@H](O)[C@@H]1O]

(21) Rongliflozin, represented by formula (21):

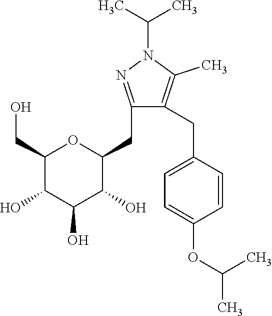 [SMILES: Cc1c(Cc2ccc(OC(C)C)cc2)c(C[C@@H]2O[C@H](CO)[C@@H](O)[C@H](O)[C@H]2O)nn1C(C)C]

(22) Wanpagliflozin;
(23) Enavogliflozin, represented by formula (23):

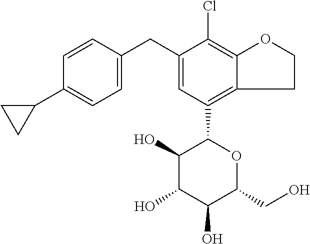 [SMILES: OC[C@H]1O[C@@H](c2cc(Cc3ccc(C4CC4)cc3)c(Cl)c3c2CCO3)[C@H](O)[C@@H](O)[C@@H]1O]

(24) TFC-039, represented by formula (24):

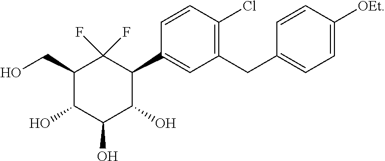 [SMILES: CCOc1ccc(Cc2cc([C@H]3[C@H](O)[C@@H](O)[C@H](O)[C@@H](CO)C3(F)F)ccc2Cl)cc1]

The term “velagliflozin” as employed herein refers to velagliflozin of the above structure as well as pharmaceutically acceptable forms thereof, including hydrates and solvates thereof, and crystalline forms thereof. The compound, methods of its synthesis and co-crystals thereof are described in WO 2007/128749, WO 2014/016381 and WO 2019/121509 for example.

The term “dapagliflozin” as employed herein refers to dapagliflozin of the above structure as well as pharmaceutically acceptable forms thereof, including hydrates and solvates thereof, and crystalline forms thereof. The compound and methods of its synthesis are described in WO 03/099836 for example. Preferred hydrates, solvates and crystalline forms are described in the patent applications WO 2008/116179 and WO 2008/002824 for example.

The term “canagliflozin” as employed herein refers to canagliflozin of the above structure as well as pharmaceutically acceptable forms thereof, including hydrates and solvates thereof, and crystalline forms thereof. The compound and methods of its synthesis are described in WO 2005/012326 and WO 2009/035969 for example. Preferred hydrates, solvates and crystalline forms are described in the patent application WO 2008/069327 for example.

The term “empagliflozin” as employed herein refers to empagliflozin of the above structure as well as pharmaceutically acceptable forms thereof, including hydrates and solvates thereof, and crystalline forms thereof. The compound and methods of its synthesis are described in WO 2005/092877, WO 2006/120208 and WO 2011/039108 for example. A preferred crystalline form is described in the patent applications WO 2006/117359 and WO 2011/039107 for example.

The term “atigliflozin” as employed herein refers to atigliflozin of the above structure as well as pharmaceutically acceptable forms thereof, including hydrates and solvates thereof, and crystalline forms thereof. The compound and methods of its synthesis are described in WO 2004/007517 for example.

The term “ipragliflozin” as employed herein refers to ipragliflozin of the above structure as well as pharmaceutically acceptable forms thereof, including hydrates and solvates thereof, and crystalline forms thereof. The compound and methods of its synthesis are described in WO 2004/080990, WO 2005/012326 and WO 2007/114475 for example.

The term “tofogliflozin” as employed herein refers to tofogliflozin of the above structure as well as pharmaceutically acceptable forms thereof, including hydrates and solvates thereof, and crystalline forms thereof. The compound and methods of its synthesis are described in WO 2007/140191 and WO 2008/013280 for example.

The term “luseogliflozin” as employed herein refers to luseogliflozin of the above structure as well as pharmaceutically acceptable forms thereof, including hydrates and solvates thereof, and crystalline forms thereof.

The term “ertugliflozin” as employed herein refers to ertugliflozin of the above structure as well as pharmaceutically acceptable forms thereof, including hydrates and solvates thereof, and crystalline forms thereof. The compound is described for example in WO 2010/023594.

The term “remogliflozin” as employed herein refers to remogliflozin of the above structure as well as pharmaceutically acceptable forms thereof, including prodrugs of remogliflozin, in particular remogliflozin etabonate, including hydrates and solvates thereof, and crystalline forms thereof. Methods of its synthesis are described in the patent applications EP 1 213 296 and EP 1 354 888 for example.

The term “sergliflozin” as employed herein refers to sergliflozin of the above structure as well as pharmaceutically acceptable forms thereof, including prodrugs of sergliflozin, in particular sergliflozin etabonate, including hydrates and solvates thereof, and crystalline forms thereof. Methods for its manufacture are described in the patent applications EP 1 344 780 and EP 1 489 089 for example.

The compound of formula (16) above, i.e., sotagliflozin, and its manufacture are described for example in WO 2008/042688 or WO 2009/014970.

The term “bexagliflozin” as employed herein refers to bexagliflozin of the above structure as well as pharmaceutically acceptable forms thereof, including hydrates and solvates thereof, and crystalline forms thereof. The compound and methods of its synthesis are described in WO 2009/026537 for example.

The term “TFC-039” as employed herein refers to the above structure as well as pharmaceutically acceptable forms thereof, including hydrates and solvates thereof, and crystalline forms thereof. The compound and methods of its synthesis are described in WO 2012/160218 for example.

Preferred SGLT-2 inhibitors are glucopyranosyl-substituted benzene derivatives. Optionally, one or more hydroxyl groups of the glucopyranosyl group in such one or more SGLT-2 inhibitors may be acylated with groups selected from (C₁₋₁₈-alkyl)carbonyl, (C₁₋₁₈-alkyl)oxycarbonyl, phenylcarbonyl and phenyl-(C₁₋₃-alkyl)-carbonyl.

More preferred are glucopyranosyl-substituted benzonitrile derivatives of formula (1) as disclosed herein above. Yet more preferred are glucopyranosyl-substituted benzonitrile derivatives of formula (18):

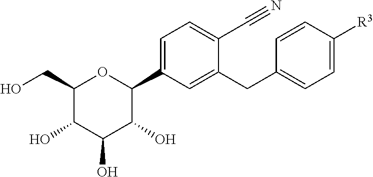

wherein
R³ denotes cyclopropyl, hydrogen, fluorine, chlorine, bromine, iodine, methyl, ethyl, propyl, isopropyl, butyl, sec-butyl, iso-butyl, tert-butyl, 3-methyl-but-1-yl, cyclobutyl, cyclopentyl, cyclohexyl, 1-hydroxy-cyclopropyl, 1-hydroxy-cyclobutyl, 1-hydroxy-cyclopentyl, 1-hydroxy-cyclohexyl, ethinyl, ethoxy, difluoromethyl, trifluoromethyl, pentafluoroethyl, 2-hydroxyl-ethyl, hydroxymethyl, 3-hydroxy-propyl, 2-hydroxy-2-methyl-prop-1-yl, 3-hydroxy-3-methyl-but-1-yl, 1-hydroxy-1-methyl-ethyl, 2,2,2-trifluoro-1-hydroxy-1-methyl-ethyl, 2,2,2-trifluoro-1-hydroxy-1-trifluoromethyl-ethyl, 2-methoxy-ethyl, 2-ethoxy-ethyl, hydroxy, difluoromethyloxy, trifluoromethyloxy, 2-methyloxy-ethyloxy, methylsulfanyl, methylsulfinyl, methlysulfonyl, ethylsulfinyl, ethylsulfonyl, trimethylsilyl, (R)-tetra-hydrofuran-3-yloxy or (S)-tetrahydrofuran-3-yloxy or cyano; and wherein R³ is preferably selected from cyclopropyl, ethyl, ethinyl, ethoxy, (R)-tetrahydrofuran-3-yloxy or (S)-tetrahydrofuran-3-yloxy; and R³ most preferably is cyclopropyl,
or a derivative thereof wherein one or more hydroxyl groups of the β-D-glucopyranosyl group are acylated with groups selected from (C₁₋₁₈-alkyl)carbonyl, (C₁₋₁₈-alkyl)oxycarbonyl, phenylcarbonyl and phenyl-(C₁₋₃-alkyl)-carbonyl.

Preferably, such SGLT-2 inhibitor is velagliflozin as shown in formula (2). Optionally, one or more hydroxyl groups of the β-D-glucopyranosyl group of velagliflozin may be acylated with groups selected from (C₁₋₁₈-alkyl)carbonyl, (C₁₋₁₈-alkyl)oxycarbonyl, phenylcarbonyl and phenyl-(C₁₋₃-alkyl)-carbonyl.

Thus, in a preferred embodiment, the at least one SGLT-2 inhibitor according to the present invention is a glucopyranosyl-substituted benzene derivative SGLT-2 inhibitor, preferably a SGLT-2 inhibitor of formula (1), more preferably of formula (18), or yet more preferably of formula (2), i.e. velagliflozin, in each case as defined herein above.

In another preferred embodiment, such SGLT-2 inhibitor is bexagliflozin as shown in formula (19). Optionally, one or more hydroxyl groups of the β-D-glucopyranosyl group of bexagliflozin may be acylated with groups selected from (C₁₋₁₈-alkyl)carbonyl, (C₁₋₁₈-alkyl)oxycarbonyl, phenylcarbonyl and phenyl-(C₁₋₃-alkyl)-carbonyl.

Thus, in another preferred embodiment, the at least one SGLT-2 inhibitor according to the present invention is a glucopyranosyl-substituted benzene derivative SGLT-2 inhibitor, preferably an SGLT-2 inhibitor of formula (19), i.e. bexagliflozin, in each case as defined herein above.

Herein, references to SGLT-2 inhibitors and/or their use according to the invention encompass pharmaceutically acceptable forms of the SGLT-2 inhibitors, unless otherwise stated.

According to the invention, any pharmaceutically acceptable form of the SGLT-2 inhibitor, e.g. of formula (1), preferably formula (18), more preferably formula (2), may be used. E.g. a crystalline form may be used. Prodrug forms are also encompassed by the present invention.

Prodrug forms may include, e.g., esters and/or hydrates. The term “prodrug” is also meant to include any covalently bonded carrier, which releases the active compound of the invention in vivo when the prodrug is administered to a mammalian subject. Prodrugs of a compound of the invention may be prepared by modifying functional groups present in the compound of the invention in such a way that the modifications are cleaved, either in routine manipulation or in vivo, to the parent compound of the invention.

Crystalline forms for use according to the invention include a complex of an SGLT-2 inhibitor with one or more amino acids (see e.g., WO 2014/016381)-so-called co-crystals. An amino acid for such use may be a natural amino acid. The amino acid may be a proteogenic amino acid (including L-hydroxyproline), or a non-proteogenic amino acid. The amino acid may be a D-or an L-amino acid. In some preferred embodiments, the amino acid is proline (L-proline and/or D-proline, preferably L-proline). E.g., a crystalline complex/co-crystal of velagliflozin with proline (e.g., L-proline) and crystalline water is preferred.

Thus, herein is disclosed a crystalline complex/co-crystal between one or more natural amino acids and an SGLT-2 inhibitor, e.g., a crystalline complex/co-crystal between one or more natural amino acids and a glucopyranosyl-substituted benzene derivative SGLT-2 inhibitor, preferably a SGLT-2 inhibitor of formula (1), more preferably of formula (18) or yet more preferably of formula (2) (velagliflozin).

A certain pharmaceutical activity is the basic prerequisite to be fulfilled by a pharmaceutically active agent before it is approved as a medicament on the market. However, there are a variety of additional requirements a pharmaceutically active agent has to comply with. These requirements are based on various parameters, which are connected with the nature of the active substance itself. Without being restrictive, examples of these parameters are the stability of the active agent under various environmental conditions, its stability during production of the pharmaceutical formulation and the stability of the active agent in the final medicament compositions. The pharmaceutically active substance used for preparing the pharmaceutical compositions should be as pure as possible and its stability in long-term storage must be guaranteed under various environmental conditions. This is essential to prevent the use of pharmaceutical compositions, which contain, in addition to the actual active substance, breakdown products thereof, for example. In such cases, the content of active substance in the medicament might be less than that specified.

Uniform distribution of the medicament in the formulation is a critical factor, particularly when the medicament has to be given in low doses. To ensure uniform distribution, the particle size of the active substance can be reduced to a suitable level, e.g., by grinding. Since breakdown of the pharmaceutically active substance as a side effect of the grinding (or micronizing) has to be avoided as far as possible, in spite of the hard conditions required during the process, it is essential that the active substance should be highly stable throughout the grinding process. Only if the active substance is sufficiently stable during the grinding process it is possible to produce a homogeneous pharmaceutical formulation, which always contains the specified amount of active substance in a reproducible manner.

Another problem, which may arise in the grinding process for preparing the desired pharmaceutical formulation, is the input of energy caused by this process and the stress on the surface of the crystals. This may in certain circumstances lead to polymorphous changes, to amorphization or to a change in the crystal lattice. Since the pharmaceutical quality of a pharmaceutical formulation requires that the active substance should always have the same crystalline morphology, the stability and properties of the crystalline active substance are subject to stringent requirements from this point of view as well.

The stability of a pharmaceutically active substance is also important in pharmaceutical compositions for determining the shelf life of the particular medicament: the shelf life is the length of time during which the medicament can be administered without any risk. High stability of a medicament in the abovementioned pharmaceutical compositions under various storage conditions is therefore an additional advantage for both the patient and the manufacturer.

The absorption of moisture reduces the content of pharmaceutically active substance because of the increased weight caused by the uptake of water. Pharmaceutical compositions with a tendency to absorb moisture have to be protected from moisture during storage, e.g., by the addition of suitable drying agents or by storing the drug in an environment where it is protected from moisture. Preferably, therefore, a pharmaceutically active substance should be at best slightly hy groscopic.

Furthermore, the availability of a well-defined crystalline form allows the purification of the drug substance by recrystallization.

Apart from the requirements indicated above, it should be generally borne in mind that any change to the solid state of a pharmaceutical composition, which is capable of improving its physical and chemical stability, gives a significant advantage over less stable forms of the same medicament.

A crystalline complex/co-crystal between a natural amino acid and an SGLT-2 inhibitor (e.g. a glucopyranosyl-substituted benzene derivative or a SGLT-2 inhibitor of formula (1), or formula (18) or, particularly, of formula (2), i.e. velagliflozin) fulfills important requirements mentioned hereinbefore.

SGLT-2 inhibitors or pharmaceutically acceptable forms thereof for use according to the invention may be prepared as pharmaceutical compositions. They may be prepared as solid or as liquid formulations. In either case, they are preferably prepared for oral administration, preferably in liquid form for oral administration (see e.g., WO 2017/032799). The SGLT-2 inhibitors or pharmaceutically acceptable forms thereof may, however, also be prepared, e.g., for parenteral administration. Solid formulations include tablets, granular forms, and other solid forms such as suppositories. Among solid formulations, tablets and granular forms are preferred

Pimobendan for use according to the invention may be prepared as pharmaceutical compositions. It may be prepared as solid or as liquid formulations. In either case, it is preferably prepared for oral administration, preferably in solid form (tablets) for oral administration (see e.g., WO 2005/084647 or WO 2015/082389). Pharmaceutical compositions within the meaning of the present invention may comprise one or more SGLT-2 inhibitors or pharmaceutically acceptable forms thereof according to the present invention in combination with pimobendan and/or telmisartan or pharmaceutically acceptable forms thereof and one or more excipients. Any excipient that allows for, or supports, the intended medical effect may be used. Such excipients are available to the skilled person. Useful excipients are for example anti-adherents (used to reduce the adhesion between the powder (granules) and the punch faces and thus prevent sticking to tablet punches), binders (solution binders or dry binders that hold the ingredients together), coatings (to protect tablet ingredients from deterioration by moisture in the air and make large or unpleasant-tasting tablets easier to swallow), disintegrants (to allow the tablet to break upon dilution), fillers, diluents, flavours, colours, glidants (flow regulators—to promote powder flow by reducing interparticle friction and cohesion), lubricants (to prevent ingredients from clumping together and from sticking to the tablet punches or capsule filling machine), preservatives, sorbents, sweeteners etc.

Formulations according to the invention, e.g., solid formulations, may comprise carriers and/or disintegrants selected from the group of sugars and sugar alcohols, e.g. mannitol, lactose, starch, cellulose, microcrystalline cellulose and cellulose derivatives, e.g. methylcellulose, and the like.

Manufacturing procedures for formulations suitable for canines are known to the person skilled in the art, and for solid formulations comprise, e.g., direct compression, dry granulation and wet granulation. In the direct compression process, the active ingredient and all other excipients are placed together in a compression apparatus that is directly applied to press tablets out of this material. The resulting tablets can optionally be coated afterwards in order to protect them physically and/or chemically, e.g., by a material known from the state of the art.

A unit for administration, e.g., a single liquid dose or a unit of a solid formulation, e.g., a tablet, may comprise 0.1 mg to 10 mg, or e.g., 0.3 mg to 1 mg, 1 mg to 3 mg, 3 mg to 10 mg: or 5 to 2500 mg, or e.g., 5 to 2000 mg, 5 mg to 1500 mg, 10 mg to 1500 mg, 10 mg to 1000 mg, or 10-500 mg of an SGLT-2 inhibitor as well as 0.1 mg to 10 mg pimobendan, or e.g., 1.25 mg, 2.5 mg, 5 mg or 10 mg pimobendan for use according to the invention. As the skilled person would understand, the content of the SGLT-2 inhibitor or pharmaceutically acceptable forms thereof and pimobendan and/or telmisartan or pharmaceutically acceptable forms thereof in a solid formulation, or any formulation as disclosed herein for administration to a non-human mammal, may be increased or decreased as appropriate in proportion to the body weight of the non-human mammal to be treated.

In one embodiment, a pharmaceutical composition for use according to the invention is designed for oral or parenteral administration, preferably for oral administration. Especially the oral administration is ameliorated by excipients, which modify the smell and/or haptic properties of the pharmaceutical composition for the intended patient, e.g., as described.

When the SGLT-2 inhibitor or pharmaceutically acceptable forms thereof in combination with pimobendan and/or telmisartan or pharmaceutically acceptable forms thereof for use according to the invention is formulated for oral administration, it is preferred that excipients confer properties, e.g., palatability and/or chewability that render the formulation suitable for administration to a non-human mammal.

Also preferred are liquid formulations. Liquid formulations may be, e.g., solutions, syrups or suspensions.

They may be administered directly to the non-human mammals or may be mixed with the food and/or drink (e.g., drinking water, or the like) of the non-human mammal. One advantage of a liquid formulation (similar to a formulation in granular form), is that such a dosage form allows precise dosing. For example, the SGLT-2 inhibitor or pharmaceutically acceptable forms thereof and pimobendan and/or telmisartan or pharmaceutically acceptable forms thereof may be dosed precisely in proportion to the body mass of a non-human mammal. Typical compositions of liquid formulations are known to the person skilled in the art.

According to the present invention two or more pharmaceutical active substances can be combined in one single dosage form, i.e., as combination drugs. The advantage of such a formulation is that the doses are fixed in this pharmaceutical formulation, i.e., available in certain fixed doses. In such a case the pharmaceutical formulation is called a “fixed-dose-combination” (FDC), which can be either a solid or a liquid formulation.

In another embodiment, a pharmaceutical composition for use according to the invention is a fixed-dose-combination (FDC) of the one or more SGLT-2 inhibitors or pharmaceutically acceptable forms thereof and pimobendan and/or telmisartan or pharmaceutically acceptable forms thereof, wherein preferably such FDC is a solid or a liquid formulation.

In a preferred embodiment, the FDC comprises velagliflozin or pharmaceutically acceptable forms thereof and pimobendan as the only pharmaceutical active substances. In another embodiment, the FDC comprises velagliflozin or pharmaceutically acceptable forms thereof and pimobendan and telmisartan or pharmaceutically acceptable forms thereof as the only pharmaceutical active substances. In another embodiment, the FDC comprises velagliflozin or pharmaceutically acceptable forms thereof and telmisartan or pharmaceutically acceptable forms thereof as the only pharmaceutical active substances.

In another preferred embodiment, the FDC comprises bexagliflozin or pharmaceutically acceptable forms thereof and pimobendan as the only pharmaceutical active substances. In another embodiment, the FDC comprises bexagliflozin or pharmaceutically acceptable forms thereof and pimobendan and telmisartan or pharmaceutically acceptable forms thereof as the only pharmaceutical active substances. In another embodiment, the FDC comprises bexagliflozin or pharmaceutically acceptable forms thereof and telmisartan or pharmaceutically acceptable forms thereof as the only pharmaceutical active substances.

A practitioner skilled in the art can determine suitable doses for the uses of the present invention. Preferred units dosing units include mg/kg bodyweight, i.e. mg SGLT-2 inhibitor per body mass of the non-human mammal. An SGLT-2 inhibitor of the invention may, e.g., be administered in doses of 0.01-10 mg/kg bodyweight per day, e.g., 0.01-5 mg/kg bodyweight per day, e.g., 0.01-4 mg/kg bodyweight per day, e.g,. 0.01-3 mg/kg bodyweight per day, e.g., 0.01-2 mg/kg bodyweight per day, e.g., 0.01-1.5 mg/kg bodyweight per day, e.g., 0.01-1 mg/kg bodyweight per day, e.g., 0.01-0.75 mg/kg bodyweight per day, e.g., 0.01-0.5 mg/kg bodyweight per day, e.g. 0.01-0.4 mg/kg bodyweight per day; or 0.1 to 3.0 mg/kg bodyweight per day, preferably from 0.2 to 2.0 mg/kg bodyweight per day, more preferably from 0.1 to 1 mg/kg bodyweight per day or from 0.5 to 1 mg/kg bodyweight per day. In another preferred embodiment, the dose is 0.01-1 mg/kg bodyweight per day, preferably 0.01-0.5 mg/kg bodyweight per day, more preferably 0.02-0.4 mg/kg bodyweight per day, e.g. 0.03-0.3 mg/kg bodyweight per day. Alternatively, an SGLT-2 inhibitor of the invention may, e.g., be administered in doses of 0.1-10 mg/kg bodyweight per day, e.g., 0.1-5 mg/kg bodyweight per day, e.g., 0.1-4 mg/kg body-weight per day, e.g., 0.1-3 mg/kg bodyweight per day, e.g., 0.1-2 mg/kg bodyweight per day, e.g,. 0.1-1.5 mg/kg bodyweight per day, e.g., 0.1-1 mg/kg bodyweight per day, e.g., 0.1-0.75 mg/kg bodyweight per day, e.g. 0.1-0.5 mg/kg bodyweight per day, e.g., 0.1-0.4 mg/kg bodyweight per day.

A practitioner skilled in the art is able to prepare an SGLT-2 inhibitor of the invention for administration according to a desired dose.

As regards pimobendan, a practitioner skilled in the art can determine suitable doses for the uses of the present invention. Preferred units dosing units include mg/kg bodyweight, i.e., mg pimobendan per body mass of the non-human mammal. Pimobendan may, e.g., be administered in doses of 0.1-1 mg/kg bodyweight per day, e.g., 0.2-0.6 mg/kg bodyweight per day, e.g., 0.5 mg/kg bodyweight per day.

A practitioner skilled in the art is able to prepare pimobendan for administration according to a desired dose.

As regards telmisartan or pharmaceutically acceptable forms thereof, a practitioner skilled in the art can determine suitable doses for the uses of the present invention. Preferred units dosing units include mg/kg body-weight, i.e., mg telmisartan per body mass of the non-human mammal. Telmisartan or pharmaceutically acceptable forms thereof may, e.g., be administered in doses of 0.01 to 10 mg/kg of bodyweight per day, preferably is 0.05 to 8 mg/kg of bodyweight, even more preferably 0.1 to 5 mg/kg of bodyweight, even more preferably 0.2 to 4 mg/kg of bodyweight, even more preferably 0.3 to 3 mg/kg of bodyweight, even more preferably 0.4 to 2.5 mg/kg of bodyweight, even more preferably 0.5 to 2 mg/kg of bodyweight, most preferred 0.75 to 1.5 mg/kg of bodyweight per day, or e.g., 1.25 mg, 2.5 mg, 5 mg or 10 mg per day.

A practitioner skilled in the art is able to prepare telmisartan or pharmaceutically acceptable forms thereof for administration according to a desired dose.

### EXAMPLES

The following examples serve to further illustrate the present invention; but the same should not be construed as a limitation of the scope of the invention disclosed herein.

### Example 1 Field Trial Evaluating Single and Combined Treatment of Once Daily Velagliflozin Administered Orally With Twice Daily Oral Pimobendan in Dogs With DCM (Different Disease Stages From B1 Onwards)

Dogs are randomized to either velagliflozin, or pimobendan, or velagliflozin and pimobendan. During the study period dogs are evaluated regularly including a full physical exam including body weight, body condition score, blood pressure and an auscultation of the cardiovascular system (e.g., frequency, rhythm, intensity, abnormalities such as murmurs), thoracic radiographs (latero-lateral, ventro-dorsal), echocardiography, ECG, blood [chemistry, complete blood count (CBC), cardiac biomarkers, ketone bodies] and urine evaluation.

The following parameters are assessed:
Clinical signs (e.g., breathlessness, dyspnoea, cough, depression, exercise intolerance, inappetence, syncope, abdominal distention, and polydipsia).
Echocardiographic parameters (e.g., IVSd, LA diameter, Ao diameter, LVIDd, LVWd, LVWs, IVSd, LVPWd, EDV, ESV, EF, % FS, presence of effusion).
ECG evaluation (optional)
Time to event (death or hospitalization for heart failure)
Number of events (death or hospitalization for heart failure, congestive heart failure (pulmonary oedema))
Reduction of Dose of diuretic treatment (e.g., furosemide, torasemide) dose
Start of additional treatment for cardiac disease (e.g., pimobendan, ACE inhibitors)
Blood parameters (e.g. NTproBNP, Troponin)
Quality of life (owner assessment)
Investigator assessment on treatment effect/disease control
Resting respiratory rate
X-ray (heart size and pulmonary edema, congestion)
Disease progression (progression to next stage)
Systolic blood pressure (SBP)
NYHA class/ACVIM class

The results of the clinical field trial show a significant and clinically relevant prolongation of survival time and the time to event (event is defined as cardiac death/euthanasia and (re-)occurrence of congestive heart failure) of the combined treatment compared to treatment with either velagliflozin or pimobendan. Additionally, clinical parameters (e.g., appetite, activity level and breathing), cardiac echoparameters and cardiac biomarkers show a higher degree of improvement compared to single treatment.

### Example 2 Field Trial Evaluating Velagliflozin as an Add-On Treatment to Pimobendan Compared to Placebo as an Add-On Treatment to Pimobendan in Dogs With DCM (Different Disease Stages from B1 Onwards)

Dogs are randomized to either placebo and pimobendan or velagliflozin and pimobendan. During the study period dogs are evaluated regularly including a full physical exam including body weight, body condition score, blood pressure and an auscultation of the cardiovascular system (e.g., frequency, rhythm, intensity, abnormalities such as murmurs), thoracic radiographs (latero-lateral, ventro-dorsal), echocardiography, ECG, blood (chemistry, CBC, cardiac biomarkers, ketone bodies) and urine evaluation.

The following parameters are assessed:
Clinical signs (e.g., breathlessness, dyspnoea, cough, depression, exercise intolerance, inappetence, syncope, abdominal distention, and polydipsia)
Echocardiographic parameters (e.g., IVSd, LA diameter, Ao diameter, LVIDd, LVWd, LVWs, IVSd, LVPWd, EDV, ESV, EF, % FS, presence of effusion)
ECG evaluation (optional)
Time to event (death or hospitalization for heart failure)
Number of events (death or hospitalization for heart failure, congestive heart failure (pulmonary oedema))
Reduction of Dose of diuretic treatment (e.g., furosemide, torasemide) dose
Start of additional treatment for cardiac disease (e.g., pimobendan, ACE-inhibitors)
Blood parameters (e.g., NTproBNP, Troponin)
Quality of life (owner assessment)
Investigator assessment on treatment effect/disease control
Resting respiratory rate
X-ray (heart size and pulmonary edema, congestion)
Disease progression (progression to next stage)
Systolic blood pressure (SBP)
NYHA-class/ACVIM class

The results of the clinical field trial show a significant and clinically relevant prolongation of survival time and the time to event (event was defined as cardiac death/euthanasia and (re-)occurrence of congestive heart failure) for the combination of velagliflozin and pimobendan compared to placebo on top of pimobendan. Additionally, clinical parameters (e.g., appetite, activity level and breathing), cardiac echoparameters and cardiac biomarkers show a higher degree of improvement compared to single treatment.

### Example 3: Field Trial Evaluating Single and Combined Treatment of Once Daily Velagliflozin Administered Orally With Once Daily Oral Telmisartan in Dogs with DCM (Different Disease Stages from B1 Onwards)

Dogs are randomized to either velagliflozin, or telmisartan, or velagliflozin and telmisartan. During the study period dogs are evaluated regularly including a full physical exam including body weight, body condition score, blood pressure and an auscultation of the cardiovascular system (e.g., frequency, rhythm, intensity, abnormalities such as murmurs), thoracic radiographs (latero-lateral, ventro-dorsal), echocardiography, ECG, blood (chemistry, CBC, cardiac biomarkers, ketone bodies) and urine evaluation.

The following parameters are assessed:
Clinical signs (e.g., breathlessness, dyspnoea, cough, depression, exercise intolerance, inappetence, syncope, abdominal distention, and polydipsia)
Echocardiographic parameters (e.g., IVSd, LA diameter, Ao diameter, LVIDd, LVWd, LVWs, IVSd, LVPWd, EDV, ESV, EF, % FS, presence of effusion)
ECG evaluation (optional)
Time to event (death or hospitalization for heart failure)
Number of events (death or hospitalization for heart failure, congestive heart failure (pulmonary oedema)).
Reduction of Dose of diuretic treatment (e.g., furosemide, torasemide) dose
Start of additional treatment for cardiac disease (e.g., pimobendan, ACE-inhibitors)
Blood parameters (e.g., NTproBNP, Troponin)
Quality of life (owner assessment)
Investigator assessment on treatment effect/disease control
Resting respiratory rate
X-ray (heart size and pulmonary edema, congestion)
Disease progression (progression to next stage)
Systolic blood pressure (SBP)
NYHA-class/ACVIM class

The results of the clinical field trial show a significant and clinically relevant prolongation of survival time and the time to event (event was defined as cardiac death/euthanasia and (re-)occurrence of congestive heart failure), prevention and improvement of hypertension of the combined treatment compared to treatment with either velagliflozin or telmisartan. Additionally, clinical parameters (e.g., appetite, activity level and breathing), cardiac echoparameters and cardiac biomarkers show a higher degree of improvement compared to single treatment.

### Example 4: Field Trial Evaluating Velagliflozin as an Add-On Treatment to Telmisartan Compared to Placebo as an Add-On Treatment to Telmisartan in Dogs with DCM (Different Disease Stages From B1 Onwards)

Dogs are randomized to either placebo and telmisartan or velagliflozin and telmisartan. During the study period dogs are evaluated regularly including a full physical exam including body weight, body condition score, blood pressure and an auscultation of the cardiovascular system (e.g., frequency, rhythm, intensity, abnormalities such as murmurs), thoracic radiographs (latero-lateral, ventro-dorsal), echocardiography, ECG, blood (chemistry, CBC, cardiac biomarkers, ketone bodies) and urine evaluation.

The following parameters are assessed:
Clinical signs (e.g., breathlessness, dyspnoea, cough, depression, exercise intolerance, inappetence, syncope, abdominal distention, and polydipsia)
Echocardiographic parameters (e.g., IVSd, LA diameter, Ao diameter, LVIDd, LVWd, LVWs, IVSd, LVPWd, EDV, ESV, EF, % FS, presence of effusion)
ECG evaluation (optional)
Time to event (death or hospitalization for heart failure)
Number of events (death or hospitalization for heart failure, congestive heart failure (pulmonary oedema))
Reduction of Dose of diuretic treatment (e.g., furosemide, torasemide) dose
Start of additional treatment for cardiac disease (e.g. pimobendan, ACE-inhibitors)
Blood parameters (e.g., NTproBNP, Troponin)
Quality of life (owner assessment)
Investigator assessment on treatment effect/disease control
Resting respiratory rate
X-ray (heart size and pulmonary edema, congestion)
Disease progression (progression to next stage)
Systolic blood pressure (SBP)
NYHA-class/AC VIM class

The results of the clinical field trial show a significant and clinically relevant prolongation of survival time and the time to event (event was defined as cardiac death/euthanasia and (re-)occurrence of congestive heart failure), prevention and improvement of hypertension for the combination of velagliflozin and telmisartan compared to placebo on top of telmisartan. Additionally, clinical parameters (e.g. appetite, activity level and breathing), cardiac echoparameters and cardiac biomarkers show a higher degree of improvement compared to single treatment.

### Example 5: Field Trial Evaluating Single and Combined Treatment of Once Daily Velagliflozin Administered Orally With Twice Daily Oral Pimobendan in Dogs With MVD (Different Disease Stages From B1 Onwards)

Dogs are randomized to either velagliflozin, or pimobendan, or velagliflozin and pimobendan. During the study period dogs are evaluated regularly including a full physical exam including body weight, body condition score, blood pressure and an auscultation of the cardiovascular system (e.g., frequency, rhythm, intensity, abnormalities such as murmurs), thoracic radiographs (latero-lateral, ventro-dorsal), echocardiography, ECG, blood (chemistry, CBC, cardiac biomarkers, ketone bodies) and urine evaluation.

The following parameters are assessed:
Clinical signs (e.g., breathlessness, dyspnoea, cough, depression, exercise intolerance, inappetence, syncope, abdominal distention, and polydipsia)
Echocardiographic parameters (e.g., IVSd, LA diameter, Ao diameter, LVIDd, LVWd, LVWs, IVSd, LVPWd, EDV, ESV, EF, % FS, presence of effusion)
ECG evaluation (optional)
Time to event (death or hospitalization for heart failure)
Number of events (death or hospitalization for heart failure, congestive heart failure (pulmonary oedema))
Reduction of Dose of diuretic treatment (e.g., furosemide, torasemide) dose
Start of additional treatment for cardiac disease (e.g., pimobendan, ACE-inhibitors)
Blood parameters (e.g. NTproBNP, Troponin)
Quality of life (owner assessment)
Investigator assessment on treatment effect/disease control
Resting respiratory rate
X-ray (heart size and pulmonary edema, congestion)
Disease progression (progression to next stage)
Systolic blood pressure (SBP)
NYHA-class/ACVIM class

The results of the clinical field trial show a significant and clinically relevant prolongation of survival time and the time to event (event was defined as cardiac death/euthanasia and (re-)occurrence of congestive heart failure) of the combined treatment compared to treatment with either velagliflozin or pimobendan. Additionally, clinical parameters (e.g., appetite, activity level and breathing), cardiac echoparameters and cardiac biomarkers show a higher degree of improvement compared to single treatment.

### Example 6: Field Trial Evaluating Velagliflozin as an Add-On Treatment to Pimobendan Compared to Placebo as an Add-On Treatment to Pimobendan in Dogs with MVD (Different Disease Stages From B1 Onwards)

Dogs are randomized to either placebo and pimobendan or velagliflozin and pimobendan. During the study period dogs are evaluated regularly including a full physical exam including body weight, body condition score, blood pressure and an auscultation of the cardiovascular system (e.g., frequency, rhythm, intensity, abnormalities such as murmurs), thoracic radiographs (latero-lateral, ventro-dorsal), echocardiography, ECG, blood (chemistry, CBC, cardiac biomarkers, ketone bodies) and urine evaluation.

The following parameters are assessed:
Clinical signs (e.g., breathlessness, dyspnoea, cough, depression, exercise intolerance, inappetence, syncope, abdominal distention, and polydipsia)
Echocardiographic parameters (e.g., IVSd, LA diameter, Ao diameter, LVIDd, LVWd, LVWs, IVSd, LVPWd, EDV, ESV, EF, % FS, presence of effusion)
ECG evaluation (optional)
Time to event (death or hospitalization for heart failure)
Number of events (death or hospitalization for heart failure, congestive heart failure (pulmonary oedema)).
Reduction of Dose of diuretic treatment (e.g., furosemide, torasemide) dose
Start of additional treatment for cardiac disease (e.g., pimobendan, ACE-inhibitors)
Blood parameters (e.g., NTproBNP, Troponin)
Quality of life (owner assessment)
Investigator assessment on treatment effect/disease control
Resting respiratory rate
X-ray (heart size and pulmonary edema, congestion)
Disease progression (progression to next stage)
Systolic blood pressure (SBP)
NYHA-class/ACVIM class

The results of the clinical field trial show a significant and clinically relevant prolongation of survival time and the time to event (event was defined as cardiac death/euthanasia and (re-)occurrence of congestive heart failure) for the combination of velagliflozin and pimobendan compared to placebo on top of pimobendan. Additionally, clinical parameters (e.g., appetite, activity level and breathing), cardiac echoparameters and cardiac biomarkers show a higher degree of improvement compared to single treatment.

### Example 7: Field Trial Evaluating Single and Combined Treatment of Once Daily Velagliflozin Administered Orally With Once Daily Oral Telmisartan in Dogs With MVD (Different Disease Stages from B1 Onwards)

Dogs are randomized to either velagliflozin, or telmisartan, or velagliflozin and telmisartan. During the study period dogs are evaluated regularly including a full physical exam including body weight, body condition score, blood pressure and an auscultation of the cardiovascular system (e.g., frequency, rhythm, intensity, abnormalities such as murmurs), thoracic radiographs (latero-lateral, ventro-dorsal), echocardiography, ECG, blood (chemistry, CBC, cardiac biomarkers, ketone bodies) and urine evaluation.

The following parameters are assessed:
Clinical signs (e.g., breathlessness, dyspnoea, cough, depression, exercise intolerance, inappetence, syncope, abdominal distention, and polydipsia)
Echocardiographic parameters (e.g., IVSd, LA diameter, Ao diameter, LVIDd, LVWd, LVWs, IVSd, LVPWd, EDV, ESV, EF, % FS, presence of effusion)
ECG evaluation (optional)
Time to event (death or hospitalization for heart failure)
Number of events (death or hospitalization for heart failure, congestive heart failure (pulmonary oedema))
Reduction of Dose of diuretic treatment (e.g., furosemide, torasemide) dose
Start of additional treatment for cardiac disease (e.g., pimobendan, ACE-inhibitors)
Blood parameters (e.g., NTproBNP, Troponin)
Quality of life (owner assessment)
Investigator assessment on treatment effect/disease control
Resting respiratory rate
X-ray (heart size and pulmonary edema, congestion)
Disease progression (progression to next stage)
Systolic blood pressure (SBP)
NYHA-class/ACVIM class

The results of the clinical field trial show a significant and clinically relevant prolongation of survival time and the time to event (event was defined as cardiac death/euthanasia and (re-)occurrence of congestive heart failure), prevention of hypertension, improvement of hypertension of the combined treatment compared to treatment with either velagliflozin or telmisartan. Additionally, clinical parameters (e.g. appetite, activity level and breathing), cardiac echoparameters and cardiac biomarkers show a higher degree of improvement compared to single treatment.

### Example 8: Field Trial Evaluating Velagliflozin as an Add-On Treatment to Telmisartan Compared to Placebo as an Add-On Treatment to Telmisartan in Dogs With MVD (Different Disease Stages from B1 Onwards)

Dogs are randomized to either placebo and telmisartan or velagliflozin and telmisartan. During the study period dogs are evaluated regularly including a full physical exam including body weight, body condition score, blood pressure and an auscultation of the cardiovascular system (e.g. frequency, rhythm, intensity, abnormalities such as murmurs), thoracic radiographs (latero-lateral, ventro-dorsal), echocardiography, ECG, blood (chemistry, CBC, cardiac biomarkers, ketone bodies) and urine evaluation.

The following parameters are assessed:
Clinical signs (e.g., breathlessness, dyspnoea, cough, depression, exercise intolerance, inappetence, syncope, abdominal distention, and polydipsia)
Echocardiographic parameters (e.g., IVSd, LA diameter, Ao diameter, LVIDd, LVWd, LVWs, IVSd, LVPWd, EDV, ESV, EF, % FS, presence of effusion)
ECG evaluation (optional)
Time to event (death or hospitalization for heart failure)
Number of events (death or hospitalization for heart failure, congestive heart failure (pulmonary oedema))
Reduction of Dose of diuretic treatment (e.g., furosemide, torasemide) dose
Start of additional treatment for cardiac disease (e.g. pimobendan, ACE-inhibitors)
Blood parameters (e.g., NTproBNP, Troponin)
Quality of life (owner assessment)
Investigator assessment on treatment effect/disease control
Resting respiratory rate
X-ray (heart size and pulmonary edema, congestion)
Disease progression (progression to next stage)
Systolic blood pressure (SBP)
NYHA-class/ACVIM class

The results of the clinical field trial show a significant and clinically relevant prolongation of survival time and the time to event (event was defined as cardiac death/euthanasia and (re-)occurrence of congestive heart failure), prevention and improvement of hypertension for the combination of velagliflozin and telmisartan compared to placebo on top of telmisartan. Additionally, clinical parameters (e.g., appetite, activity level and breathing), cardiac echoparameters and cardiac biomarkers show a higher degree of improvement compared to single treatment.

### Exampl 9: Field Trial Evaluating Single and Combined Treatment of Once Daily Velagliflozin Administered Orally With Twice Daily Oral Pimobendan in Cats With HCM (Different Disease Stages from B1 Onwards)

Cats are randomized to either velagliflozin, or pimobendan, or velagliflozin and pimobendan. During the study period cats are evaluated regularly including a full physical exam including body weight, body condition score, blood pressure and an auscultation of the cardiovascular system (e.g. frequency, rhythm, intensity, abnormalities such as murmurs), thoracic radiographs (latero-lateral, ventro-dorsal), echocardiography, ECG, blood (chemistry, CBC, cardiac biomarkers, ketone bodies) and urine evaluation.

The following parameters are assessed:
Clinical signs (e.g., breathlessness, dyspnoea, cough, depression, exercise intolerance, inappetence, syncope, abdominal distention, and polydipsia)
Echocardiographic parameters (e.g., IVSd, LA diameter, Ao diameter, LVIDd, LVWd, LVWs, IVSd, LVPWd, EDV, ESV, EF, % FS, presence of effusion)
ECG evaluation (optional)
Time to event (death or hospitalization for heart failure)
Number of events (death or hospitalization for heart failure, congestive heart failure (pulmonary oedema))
Reduction of Dose of diuretic treatment (e.g., furosemide, torasemide) dose
Start of additional treatment for cardiac disease (e.g., pimobendan, ACE-inhibitors)
Blood parameters (e.g., NTproBNP, Troponin)
Quality of life (owner assessment)
Investigator assessment on treatment effect/disease control
Resting respiratory rate
X-ray (heart size and pulmonary edema, congestion)
Disease progression (progression to next stage)
Systolic blood pressure (SBP)
NYHA-class/ACVIM class

The results of the clinical field trial show a significant and clinically relevant prolongation of survival time and the time to event (event was defined as cardiac death/euthanasia and (re-)occurrence of congestive heart failure) of the combined treatment compared to treatment with either velagliflozin or pimobendan. Additionally, clinical parameters (e.g., appetite, activity level and breathing), cardiac echoparameters and cardiac biomarkers show a higher degree of improvement compared to single treatment.

### Example 10: Field Trial Evaluating Velagliflozin as an Add-On Treatment to Pimobendan Compared to Placebo as an Add-On Treatment to Pimobendan in Cats With HCM (Different Disease Stages from B1 Onwards)

Cats are randomized to either placebo and pimobendan or velagliflozin and pimobendan. During the study period cats will be evaluated regularly including a full physical exam including body weight, body condition score, blood pressure and an auscultation of the cardiovascular system (e.g., frequency, rhythm, intensity, abnormalities such as murmurs), thoracic radiographs (latero-lateral, ventro-dorsal), echocardiography, ECG, blood (chemistry, CBC, cardiac biomarkers, ketone bodies) and urine evaluation.

The following parameters are assessed:
Clinical signs (e.g., breathlessness, dyspnoea, cough, depression, exercise intolerance, inappetence, syncope, abdominal distention, and polydipsia)
Echocardiographic parameters (e.g. IVSd, LA diameter, Ao diameter, LVIDd, LVWd, LVWs, IVSd, LVPWd, EDV, ESV, EF, % FS, presence of effusion)
ECG evaluation (optional)
Time to event (death or hospitalization for heart failure)
Number of events (death or hospitalization for heart failure, congestive heart failure (pulmonary oedema))
Reduction of Dose of diuretic treatment (e.g., furosemide, torasemide) dose
Start of additional treatment for cardiac disease (e.g., pimobendan, ACE-inhibitors)
Blood parameters (e.g., NTproBNP, Troponin)
Quality of life (owner assessment)
Investigator assessment on treatment effect/disease control
Resting respiratory rate
X-ray (heart size and pulmonary edema, congestion)
Disease progression (progression to next stage)
Systolic blood pressure (SBP)
NYHA-class/AC VIM class

The results of the clinical field trial show a significant and clinically relevant prolongation of survival time and the time to event (event was defined as cardiac death/euthanasia and (re-)occurrence of congestive heart failure) for the combination of velagliflozin and pimobendan compared to placebo on top of pimobendan. Additionally, clinical parameters (e.g., appetite, activity level and breathing), cardiac echoparameters and cardiac biomarkers show a higher degree of improvement compared to single treatment.

### Example 11: Field Trial Evaluating Single and Combined Treatment of Once Daily Velagliflozin Administered Orally With Once Daily Oral Telmisartan in Cats With HCM (Different Disease Stages From B1 Onwards)

Cats are randomized to either velagliflozin, or telmisartan, or velagliflozin and telmisartan. During the study period cats are evaluated regularly including a full physical exam including body weight, body condition score, blood pressure and an auscultation of the cardiovascular system (e.g., frequency, rhythm, intensity, abnormalities such as murmurs), thoracic radiographs (latero-lateral, ventro-dorsal), echocardiography, ECG, blood (chemistry, CBC, cardiac biomarkers, ketone bodies) and urine evaluation.

The following parameters are assessed: .
Clinical signs (e.g., breathlessness, dyspnoea, cough, depression, exercise intolerance, inappetence, syn-cope, abdominal distention, and polydipsia)
Echocardiographic parameters (e.g., IVSd, LA diameter, Ao diameter, LVIDd, LVWd, LVWs, IVSd, LVPWd, EDV, ESV, EF, % FS, presence of effusion)
ECG evaluation (optional)
Time to event (death or hospitalization for heart failure)
Number of events (death or hospitalization for heart failure, congestive heart failure (pulmonary oedema))
Reduction of Dose of diuretic treatment (e.g., furosemide, torasemide) dose
Start of additional treatment for cardiac disease (e.g., pimobendan, ACE-inhibitors)
Blood parameters (e.g., NTproBNP, Troponin)
Quality of life (owner assessment)
Investigator assessment on treatment effect/disease control
Resting respiratory rate
X-ray (heart size and pulmonary edema, congestion)
Disease progression (progression to next stage)
Systolic blood pressure (SBP)
NYHA-class/ACVIM class

The results of the clinical field trial show a significant and clinically relevant prolongation of survival time and the time to event (event was defined as cardiac death/euthanasia and (re-)occurrence of congestive heart failure), prevention of hypertension, improvement of hypertension of the combined treatment compared to treatment with either velagliflozin or telmisartan. Additionally, clinical parameters (e.g., appetite, activity level and breathing), cardiac echoparameters and cardiac biomarkers show a higher degree of improvement compared to single treatment.

### Example 12: Field Trial Evaluating Velagliflozin as an Add-On Treatment to Pimobendan Compared to Placebo as an Add-On Treatment to Telmisartan in Cats With HCM (Different Disease Stages From B1 Onwards)

Cats are randomized to either placebo and telmisartan or velagliflozin and telmisartan. During the study period cats are evaluated regularly including a full physical exam including body weight, body condition score, blood pressure and an auscultation of the cardiovascular system (e.g., frequency, rhythm, intensity, abnormalities such as murmurs), thoracic radiographs (latero-lateral, ventro-dorsal), echocardiography, ECG, blood (chemistry, CBC, cardiac biomarkers, ketone bodies) and urine evaluation.

The following parameters are assessed:
Clinical signs (e.g., breathlessness, dyspnoea, cough, depression, exercise intolerance, inappetence, syn-cope, abdominal distention, and polydipsia)
Echocardiographic parameters (e.g., IVSd, LA diameter, Ao diameter, LVIDd, LVWd, LVWs, IVSd, LVPWd, EDV, ESV, EF, % FS, presence of effusion)
ECG evaluation (optional)
Time to event (death or hospitalization for heart failure)
Number of events (death or hospitalization for heart failure, congestive heart failure (pulmonary oedema))
Reduction of Dose of diuretic treatment (e.g., furosemide, torasemide) dose
Start of additional treatment for cardiac disease (e.g. pimobendan, ACE-inhibitors)
Blood parameters (e.g., NTproBNP, Troponin)
Quality of life (owner assessment)
Investigator assessment on treatment effect/disease control
Resting respiratory rate
X-ray (heart size and pulmonary edema, congestion)
Disease progression (progression to next stage)
Systolic blood pressure (SBP)
NYHA-class/ACVIM class

The results of the clinical field trial show a significant and clinically relevant prolongation of survival time and the time to event (event was defined as cardiac death/euthanasia and (re-)occurrence of congestive heart failure), prevention and improvement of hypertension for the combination of velagliflozin and telmisartan compared to placebo on top of telmisartan. Additionally, clinical parameters (e.g., appetite, activity level and breathing), cardiac echoparameters and cardiac biomarkers show a higher degree of improvement compared to single treatment.

### Example 13: Field Trial Evaluating the Combined Treatment of Velagliflozin, Telmisartan and Pimobendan in Dogs With DCM (Different Disease Stages From B1 Onwards)

Dogs are randomized to either velagliflozin, or telmisartan, or pimobendan, or velagliflozin and telmisartan and pimobendan. During the study period dogs are evaluated regularly including a full physical exam including body weight, body condition score, blood pressure and an auscultation of the cardiovascular system (e.g., frequency, rhythm, intensity, abnormalities such as murmurs), thoracic radiographs (latero-lateral, ventrodorsal), echocardiography, ECG, blood (chemistry, CBC, cardiac biomarkers, ketone bodies) and urine evaluation.

The following parameters are assessed:
Clinical signs (e.g., breathlessness, dyspnoea, cough, depression, exercise intolerance, inappetence, syn-cope, abdominal distention, and polydipsia)
Echocardiographic parameters (e.g., IVSd, LA diameter, Ao diameter, LVIDd, LVWd, LVWs, IVSd, LVPWd, EDV, ESV, EF, % FS, presence of effusion)
ECG evaluation (optional)
Time to event (death or hospitalization for heart failure)
Number of events (death or hospitalization for heart failure, congestive heart failure (pulmonary oedema))
Reduction of dose of diuretic treatment (e.g., furosemide, torasemide) dose
Start of additional treatment for cardiac disease (e.g., pimobendan, ACE inhibitors)
Blood parameters (e.g., NTproBNP, Troponin)
Quality of life (owner assessment)
Investigator assessment on treatment effect/disease control
Resting respiratory rate
X-ray (heart size and pulmonary edema, congestion)
Disease progression (progression to next stage of the disease)
Systolic blood pressure (SBP)
NYHA-class/ACVIM class

The results of the clinical field trial show a significant and clinically relevant prolongation of survival time and the time to event (event was defined as cardiac death/euthanasia and (re-)occurrence of congestive heart failure), prevention and improvement of hypertension for the combination of velagliflozin, telmisartan and pimobendan compared to single treatments. Additionally, clinical parameters (e.g., appetite, activity level and breathing), cardiac echoparameters and cardiac biomarkers show a higher degree of improvement compared to single treatments.

### Example 14: Field Trial Evaluating the Combined Treatment of Velagliflozin, Telmisartan and Pimobendan in Dogs With MVD (Different Disease Stages From B1 Onwards)

Dogs are randomized to either velagliflozin, or telmisartan, or pimobendan, or velagliflozin and telmisartan and pimobendan. During the study period dogs are evaluated regularly including a full physical exam including body weight, body condition score, blood pressure and an auscultation of the cardiovascular system (e.g., frequency, rhythm, intensity, abnormalities such as murmurs), thoracic radiographs (latero-lateral, ventrodorsal), echocardiography, ECG, blood (chemistry, CBC, cardiac biomarkers, ketone bodies) and urine evaluation.

The following parameters are assessed:
Clinical signs (e.g., breathlessness, dyspnoea, cough, depression, exercise intolerance, inappetence, syn-cope, abdominal distention, and polydipsia)
Echocardiographic parameters (e.g., IVSd, LA diameter, Ao diameter, LVIDd, LVWd, LVWs, IVSd, LVPWd, EDV, ESV, EF, % FS, presence of effusion)
ECG evaluation (optional)
Time to event (death or hospitalization for heart failure)
Number of events (death or hospitalization for heart failure, congestive heart failure (pulmonary oedema))
Reduction of dose of diuretic treatment (e.g., furosemide, torasemide) dose
Start of additional treatment for cardiac disease (e.g., pimobendan, ACE inhibitors)
Blood parameters (e.g., NTproBNP, Troponin)
Quality of life (owner assessment)
Investigator assessment on treatment effect/disease control
Resting respiratory rate
X-ray (heart size and pulmonary edema, congestion)
Disease progression (progression to next stage of the disease)
Systolic blood pressure (SBP)
NYHA-class/ACVIM class

The results of the clinical field trial show a significant and clinically relevant prolongation of survival time and the time to event (event was defined as cardiac death/euthanasia and (re-)occurrence of congestive heart failure), prevention and improvement of hypertension for the combination of velagliflozin, telmisartan and pimobendan compared to single treatments. Additionally, clinical parameters (e.g., appetite, activity level and breathing), cardiac echoparameters and cardiac biomarkers show a higher degree of improvement compared to single treatments.

### Example 15: Field Trial Evaluating the Combined Treatment of Velagliflozin, Telmisartan and Pimobendan in Cats With HCM (Different Disease Stages From B1 Onwards)

Cats are randomized to either velagliflozin, or telmisartan, or pimobendan, or velagliflozin and telmisartan and pimobendan. During the study period cats are evaluated regularly including a full physical exam including body weight, body condition score, blood pressure and an auscultation of the cardiovascular system (e.g. frequency, rhythm, intensity, abnormalities such as murmurs), thoracic radiographs (latero-lateral, ventrodorsal), echocardiography, ECG, blood (chemistry, CBC, cardiac biomarkers, ketone bodies) and urine evaluation.

The following parameters are assessed:
Clinical signs (e.g., breathlessness, dyspnoea, cough, depression, exercise intolerance, inappetence, syn-cope, abdominal distention, and polydipsia)
Echocardiographic parameters (e.g., IVSd, LA diameter, Ao diameter, LVIDd, LVWd, LVWs, IVSd, LVPWd, EDV, ESV, EF, % FS, presence of effusion)
ECG evaluation (optional)
Time to event (death or hospitalization for heart failure)
Number of events (death or hospitalization for heart failure, congestive heart failure (pulmonary oedema))
Reduction of dose of diuretic treatment (e.g., furosemide, torasemide) dose
Start of additional treatment for cardiac disease (e.g., pimobendan, ACE inhibitors)
Blood parameters (e.g. NTproBNP, Troponin)
Quality of life (owner assessment)
Investigator assessment on treatment effect/disease control
Resting respiratory rate
X-ray (heart size and pulmonary edema, congestion)
Disease progression (progression to next stage of the disease)
Systolic blood pressure (SBP)
NYHA-class/AC VIM class

The results of the clinical field trial show a significant and clinically relevant prolongation of survival time and the time to event (event was defined as cardiac death/euthanasia and (re-)occurrence of congestive heart failure), prevention and improvement of hypertension for the combination of velagliflozin, telmisartan and pimobendan compared to single treatments. Additionally, clinical parameters (e.g. appetite, activity level and breathing), cardiac echoparameters and cardiac biomarkers show a higher degree of improvement compared to single treatments.

### Example 16: Further Field Trials

### Field Ttrial Evaluating a Combined Treatment of Once Daily Velagliflozin Administered Orally With Twice Daily Oral Pimobendan in Dogs With Dilated Cardiomyopathy (DCM)

The described study is a prospective, baseline controlled, unblinded, open-label, multi-site exploratory clinical field trial. Client owned dogs diagnosed with DCM are randomized to either a pimobendan or a velagliflozin plus pimobendan treatment group. A number of two dogs could be successfully enrolled. The clinical field trial is further ongoing.

As part of the study, enrolled dogs are evaluated regularly including a full physical examination including bodyweight, blood pressure and an auscultation of the cardiovascular system, thoracic radiographs to assess heart size, pulmonary edema and congestion, standard echocardiography, ECG, blood and urine evaluation.

The cardiovascular history is evaluated assessing the date of DCM diagnosis and whether there is a history of heart failure. An owner questionnaire is compiled on a regular basis to provide an overview of the overall condition of the dogs, the resting respiratory rate and the exercise tolerance during the last 7 days receiving treatment.

The dogs included have to be diagnosed with DCM stage B2 or stage C via physical examination and echocardiography. Variables of interest are clinical symptoms, echocardiographic parameters, blood-or urine parameters, blood pressure, number of events (death or hospitalization for heart failure or euthanasia), time-to-event, adjustment of diuretic treatment, owner questionnaire data, x-ray evaluation, disease progression (to next stage, need for additional, prohibited cardiac treatments).

During the ongoing field trial one dog successfully reaches the Day 90±5 evaluation visit. The dog is classified as stage C/clinical DCM diseased and receives velagliflozin (0.5 mg/kg bodyweight orally once daily), furosemide (1 mg/kg bodyweight twice a day) and pimobendan (daily dose of 0.5 mg/kg bodyweight orally divided into two portions that are not necessarily equal).

The results of the evaluation confirm a positive treatment effect. The systolic blood pressure decreases from 163 mmHg (Screening) to 121 mmHg (Day 42) and 144 mmHg (Day 98). In addition, the heart murmur improves from grade 4 (Screening) to grade 3 (Day 42 and Day 98). During the time of treatment, the overall bodyweight of the dog improves noticeably (40.5 kg at the Screening, 44.2 kg at Day 42 and 43,8 kg at Day 98). The owner confirms the improved overall condition of the dog and the exercise tolerance of the last 7 days to be good instead of neutral.

The second dog included is diagnosed with DCM stage B2. The dog has just been included in the ongoing study and no data from a follow up visit are yet available.

### Field Trial Evaluating the Combined Treatment of Once Daily Velagliflozin Administered Orally With Twice Daily Oral Pimobendan in Dogs With Mitral Valve Disease (MVD)

The described study is a prospective, baseline controlled, unblinded, open-label, multi-site exploratory clinical field trial. Client owned dogs diagnosed with MVD are randomized at different sites to either a pimobendan or a velagliflozin plus pimobendan treatment group. Eight dogs could be successfully enrolled. One dog does not attend any follow up visit and is therefore excluded from further participation. Data from seven dogs are therefore presented in the following. The clinical field trial is further ongoing.

The dogs that enter the study are evaluated regularly including a full physical examination including bodyweight, blood pressure, auscultation of the cardiovascular system, thoracic radiographs to assess heart size, pulmonary edema and congestion, standard echocardiography, ECG, blood and urine evaluation.

The cardiovascular history is evaluated assessing the date of MVD diagnosis and whether there is a history of heart failure. An owner questionnaire is compiled on a regular basis during the study to provide an overview on the overall condition of the dogs, the respiratory rate per minute at resting and the exercise tolerance during the last seven days of treatment.

The dogs included must be diagnosed with MVD stage B2 or stage C via physical examination and echocardiography. Variables of interest are all clinical parameters evaluated in addition to laboratory values, blood pressure, number of events (death or hospitalization for heart failure or euthanasia), the time-to-event duration, the adjustment of diuretic treatment, owner questionnaire data, x-ray results and overall disease progression (requiring additional, prohibited cardiac treatment).

During the ongoing field trial three dogs are receiving a combined velagliflozin (0.5 mg/kg bodyweight orally once daily) and pimobendan treatment (daily dose of 0.5 mg/kg bodyweight orally divided into two portions that are not necessarily equal).

The results for the first dog included classified as ACVIM stage B2 confirm an improvement in echocardiographic parameters (Day 43 and Day 96) evaluated via the MINE scoring system. The score improves by one point from seven to six at Day 43 (remaining moderately diseased) and from six to five at Day 96. At Day 96 the score classifies the dog to be improved to a mild disease. The dog shows a positive lowering of the LA/Ao ratio from 1.77 (Screening) to 1.70 (Day 96). The overall condition and other main parameters remain stable (i.e., owner score, blood pressure and blood parameters).

The second dog is classified as ACVIM stage B2 as well. The dog shows a disease progression as expected for a severely diseased dog considering the pathology of the disease and worsened at Day 49 in the MINE score for the echocardiographic parameters (increase for two score points from seven to nine moving from moderate to a severe disease). The murmur which is diagnosed from the veterinarian at the auscultation of the heart improves from a very loud (Screening) to a loud murmur (Day 49). This is considered a positive treatment effect. The owner questionnaire confirms a consistently good overall condition and a good exercise tolerance.

The third dog classified as ACVIM stage B2 shows a positive effect at Day 55 remaining in a stable state of the MINE score for the echocardiographic parameters measured. The dog has a MINE score with 10 score points classifying the dog to be severely diseased. The LA/Ao ratio is positively lowered from 2.49 (Screening) to 2.35 (Day 55). Overall, the dog is showing a positive treatment effect assuming the standard progression of the disease at a certain severity level.

During the ongoing field trial four dogs are receiving a pimobendan only treatment (daily dose of 0.5 mg/kg bodyweight orally divided into two portions that are not necessarily equal).

In the first dog a worsening in the MINE score is seen at Day 88 (worsening from score seven to eight) The dog therewith progresses from a moderate to a severe disease state. The other parameters measured are mainly unchanged (i.e. heart murmur).

The second dog shows a stable MINE score for the echocardiographic parameters measured. The score remains with eight points (Day 55) even though the dog is classified to be severely diseased. The dog is improved in the Echo-Parameter LA/Ao 2.14 (Screening) to 1.96 (Day 55) and the dog owner also reports an improvement in the overall condition and the exercise tolerance of the dog in the last 7 days.

The third dog is improved in the overall condition of the owner score as well [change from neutral (Screening) to good (Day 48)]. The Echo-Parameter LA/Ao is measured at Day 48 to be improved from 1.60 at Screening to 1.53.

The fourth dog shows an improved owner score at Day 42. The neutral overall condition and a very poor exercise tolerance in the last 7 days before Screening was improved and the owner reports now a good overall condition and a good exercise tolerance. The Echo Parameter LA/Ao is also positively reduced from 1.88 (Screening) to 1.61 (Day 42). The MINE score reflects this improvement as well [eight score points (Screening), six score points (Day 42)].

### Overall Summary

Despite the known beneficial effects of pimobendan in dogs with cardiac disease, such as DCM and MVD, the data described in above trials indicate, that dogs treated with a combination of pimobendan and velagliflozin show improvements in additional cardiovascular parameters, which are unexpected to improve with pimobendan only treatment (e.g., reduction in systolic blood pressure, heart murmur intensity) or velagliflozin only treatment. The bodyweight remained stable in all treatment groups but showed a recognizable increase for the DCM dog receiving pimobendan and velagliflozin. Therefore, it can be concluded that the combination of a positive inotrope treatment, such as pimobendan, and an SGLT2 inhibitor, e.g., velagliflozin, provides synergistic beneficial effects for dogs with cardiac disease, such as DCM or MVD, which exceed the benefits of single therapy.

### Example 17: Laboratory Study (DCM Model)

A laboratory study is conducted to evaluate the effects of the SGLT2 inhibitor Velagliflozin with Pimobendan compared to Pimobendan alone and Velagliflozin alone in dogs with a moderate degree of tachypacing-induced heart-failure [dilated cardiomyopathy (DCM) model]. The study involves 12 beagle dogs, divided into three treatment groups, each containing four dogs, in two dosing phases (totaling eight dogs per group). The treatment groups are Pimobendan, Velagliflozin, and a combination of both (Pimobendan+Velagliflozin).

The dogs are implanted with a pacing generator in the right ventricle (Abbott-Johnson K et al., 2021) and subjected to two rounds of dosing for three months each. The heart failure is induced to a moderate degree (EF=˜40-45%) before the start of each dosing round, with the pacing rate maintained throughout the three-month dosing period. After each round, a washout period of about 21-28 days is given before the next round of dosing begins.

Various data are collected throughout the study, including 6-minute walk test, blood and urine analysis, and echocardiography data. The dogs undergo an anesthetized recovery procedure at the end of each three-month round, where systemic hemodynamics are recorded. A single dose of dobutamine is also administered to measure functional reserve at the end of each 3-month period.

The results show that both the Velagliflozin treated and combination therapy treated dogs have elevated urine glucose and lower creatinine levels, based on the mode of action of the SGLT2 inhibitor. Pimobendan does not affect either urine glucose or creatinine levels.

Comparing values indicative of systolic function and performance, enhancements (i.e., EF, FAS, Simpson EF, s′ wave, LVOT VTI, SV, CO, or SAX Strain) of ˜30% for Pimobendan and of ˜50% for the combination group are observed. In terms of diastolic function and performance, enhancements (i.e., IVRT, E, E′-ave) of ˜20% for Pimobendan and ˜35% for the combination group are observed. Velagliflozin alone provided little-to-no support to aid in enhanced contractility or improved relaxation.

Overall, all therapies prove beneficial as all groups increase their 6-minute walk test by 25-30% from baseline. Pimobendan therapy treatment elicits a clear and robust functional support for three months with enhanced systolic and diastolic performance. The combination treatment of Velagliflozin and Pimobendan clearly enhances cardiac function when compared to Velagliflozin and Pimobendan mono-treatments. The combination group has the greatest measured contractility, lowest cardiac filling pressures, lowest mean right atrial pressures, and uses the least amount of mechanical energy for each cardiac cycle.

In summary, the combination treatment of Velagliflozin and Pimobendan shows a mutual positive effect in dogs with a moderate degree of tachypacing-induced heart-failure mimicking DCM.

List of abbreviations:

| | |
|---|---|
| E | early Mitral valve inflows |
| IVRT | isovolumetric relaxation time |
| LVOT VTI | left ventricular outflow tract velocity time integral of the |
| | forward aortic velocity |
| SAX Strain | Strain in short axis view |
| SVt - echo | stroke volume [EDV - ESV] |
| CO - echo | cardiac output [SV × HR] |
| EF | ejection fraction |
| FAS | fractional area shortening [(LVAd - LVAs)/LVAd × 100] |
| Simpson EF | Using simpson's method of disks to measure EF - LAX |
| E′ - ave | the average of E′ -med and E′ - lat |
| S′ - ave | the average of s′ -med ands′ - lat |

### REFERENCES

(1) Abbott-Johnson K, et al., Front Vet Sci. 2021, 8:646437 (doi: 10.3389/fvets.2021.646437. PMID: 34277749; PMCID: PMC8281278)
(2) Atkins CE et al., J Am Vet Med Assoc. 1992, 201 (4): 613-618
(3) EP 0 008 391
(4) EP 0 502 314
(5) Freeman et al., Cardiol Res. 2017, 8 (4): 139-142
(6) Hoenig M et al., J Vet Pharmacol Therapeutics 2018, 41 (2): 266-273
(7) Lin Y et al., J Am Heart Assoc 2021, 10: e019274
(8) Little CJL et al., J Small Anim Prac 2008, 49 (1): 17-25
(9) Matsumura K et al., Cardiovascular Ultrasound 2019, 17 (1): 26
(10) McMurray JJV et al., Eur. J. Heart Fail. 2019; 21:665-675
(11) Nishinarity R et al., J Am Heart Assoc 2021, 10: e017483
(12) Payne JR et al., J Vet Intern Med. 2013, 27 (6): 1427-1436
(13) Rush JE et al., J Am Vet Med Assoc. 2002, 220 (2): 202-207
(14) Santos-Gallego CG et al., J American College Cardiol 2019, 73 (15): 1931-1944
(15) Silva Custodio Jr J et al., Heart Failure Reviews 2018, 23 (3): 409-418
(16) US 2011/098240
(17) US 2015/164856
(18) US 2016/000816
(19) US 2017/266152
(20) US 2019/076395
(21) US 2020/054656
(22) US 2021/260090
(23) US 2023/000816
(24) U.S. Pat. No. 6,358,986
(25) U.S. Pat. No. 6,410,742
(26) U.S. Pat. No. 10,537,570
(27) WO 00/043370
(28) WO 2003/037876
(29) WO 2005/084647
(30) WO 2005/092343
(31) WO 2007/054514
(32) WO 2011/153953
(33) WO 2014/016381
(34) WO 2015/082389
(35) WO 2017/032799
(36) WO 2017/174571
(37) WO 2019/059557
(38) WO 2021/092341
(39) WO 2021/165177
(40) WO 2022/036506
(41) WO 2023/006718

The following clauses are also within the spirit of the present invention and are therefore part of this disclosure. It is further within the spirit of the present invention and this disclosure to further combine the subject-matter of the following clauses with any further aspects, embodiments and/or preferred embodiments as herein disclosed.
1. One or more SGLT-2 inhibitors or pharmaceutically acceptable forms thereof in combination with pimobendan and/or telmisartan or pharmaceutically acceptable forms thereof for use as a medicament.
2. The one or more SGLT-2 inhibitors or pharmaceutically acceptable forms thereof in combination with pimobendan and/or telmisartan or pharmaceutically acceptable forms thereof for use according to clause 1 in a method of prevention and/or treatment of one or more cardiac diseases in a non-human mammal/non-human mammal patient, in particular a canine/canine patient or a feline/feline patient.
3. The one or more SGLT-2 inhibitors or pharmaceutically acceptable forms thereof in combination with pimobendan and/or telmisartan or pharmaceutically acceptable forms thereof for use according to clause 2 in a method of treatment of one or more cardiac diseases in a non-human mammal/non-human mammal patient, in particular a canine/canine patient or a feline/feline patient.
4. The one or more SGLT-2 inhibitors or pharmaceutically acceptable forms thereof in combination with pimobendan and/or telmisartan or pharmaceutically acceptable forms thereof for use according to clause 2 or clause 3, wherein the one or more cardiac diseases are selected from the group consisting of: acquired cardiac disease: congenital cardiac disease; heart failure; congestive heart failure: asymptomatic/preclinical/occult heart failure: heart failure due to (myxomatous) mitral valve disease [(M)MVD]; congestive heart failure due to (myxomatous) mitral valve disease [(M)MVD]; asymptomatic/preclinical/occult heart failure due to (myxomatous) mitral valve disease [(M)MVD]; (myxomatous) mitral valve disease [(M)MVD]; clinically overt (myxomatous) mitral valve disease [(M)MVD]; asymptomatic/preclinical/occult (myxomatous) mitral valve disease [(M)MVD]; heart failure due to dilated cardiomyopathy (DCM); congestive heart failure due to dilated cardiomyopathy (DCM); asymptomatic/preclinical/occult heart failure due to dilated cardiomyopathy (DCM); dilated cardiomyopathy (DCM); clinically overt dilated cardiomyopathy (DCM); asymptomatic/preclinical/occult dilated cardiomyopathy (DCM); aortic stenosis (valvular, supravalvular and/or subvalvular); heart failure due to one or more cardiomyopathies; heart failure due to hypertrophic cardiomyopathy (HCM); heart failure due to hypertrophic obstructive cardiomyopathy (HOCM); heart failure due to restrictive cardiomyopathy (RCM); heart failure due to dilated cardiomyopathy (DCM); heart failure due to unclassified cardiomyopathy (UCM); heart failure due to arrhythmogenic right ventricular cardiomyopathy (ARVC); hypertrophic cardiomyopathy (HCM); hypertrophic obstructive cardiomyopathy (HOCM); restrictive cardiomyopathy (RCM); dilated cardiomyopathy (DCM); unclassified cardiomyopathy (UCM); and/or arrhythmogenic right ventricular cardiomyopathy (ARVC).
5. The one or more SGLT-2 inhibitors or pharmaceutically acceptable forms thereof in combination with pimobendan and/or telmisartan or pharmaceutically acceptable forms thereof for use according to clause 4, wherein the one or more cardiac diseases are selected from the group consisting of: acquired cardiac disease; congenital cardiac disease; heart failure: congestive heart failure; asymptomatic/preclinical/occult heart failure: (myxomatous) mitral valve disease [(M)MVD]; clinically overt (myxomatous) mitral valve disease [(M)MVD]; asymptomatic/preclinical/occult (myxomatous) mitral valve disease [(M)MVD]; dilated cardiomyopathy (DCM); clinically overt dilated cardiomyopathy (DCM); asymptomatic/preclinical/occult dilated cardiomyopathy (DCM); aortic stenosis (valvular, supravalvular and/or subvalvular); and wherein the non-human mammal/non-human mammal patient is a canine/canine patient: preferably a canine patient in need of such prevention and/or treatment; more preferably a dog in need of such prevention and/or treatment, even more preferably a non-diabetic dog in need of such prevention and/or treatment.
6. The one or more SGLT-2 inhibitors or pharmaceutically acceptable forms thereof in combination with pimobendan and/or telmisartan or pharmaceutically acceptable forms thereof for use according to clause 4, wherein the one or more cardiac diseases are selected from the group consisting of: acquired cardiac disease; congenital cardiac disease; heart failure; congestive heart failure; asymptomatic/preclinical/occult heart failure; heart failure due to one or more cardiomyopathies; heart failure due to hypertrophic cardiomyopathy (HCM); heart failure due to hypertrophic obstructive cardiomyopathy (HOCM); heart failure due to restrictive cardiomyopathy (RCM); heart failure due to dilated cardiomyopathy (DCM); heart failure due to unclassified cardiomyopathy (UCM); heart failure due to arrhythmogenic right ventricular cardiomyopathy (ARVC); hypertrophic cardiomyopathy (HCM); hypertrophic obstructive cardiomyopathy (HOCM); restrictive cardiomyopathy (RCM); dilated cardiomyopathy (DCM); unclassified cardiomyopathy (UCM); and/or arrhythmogenic right ventricular cardiomyopathy (ARVC); and wherein the non-human mammal/non-human mammal patient is a feline/feline patient; preferably a feline patient in need of such prevention and/or treatment, more preferably a cat in need of such prevention and/or treatment, even more preferably a non-diabetic cat in need of such prevention and/or treatment.
7. The one or more SGLT-2 inhibitors or pharmaceutically acceptable forms thereof in combination with pimobendan and/or telmisartan or pharmaceutically acceptable forms thereof for use according to any one of clauses 1 to 6, wherein the one or more SGLT-2 inhibitors are selected from the group consisting of:
(1) a glucopyranosyl-substituted benzene derivative of the formula (1)

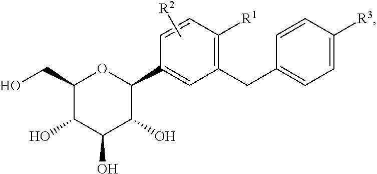

wherein R¹ denotes cyano, Cl or methyl (most preferably cyano);
R² denotes H, methyl, methoxy or hydroxy (most preferably H) and
R³ denotes cyclopropyl, hydrogen, fluorine, chlorine, bromine, iodine, methyl, ethyl, propyl, isopropyl, butyl, sec-butyl, iso-butyl, tert-butyl, 3-methyl-but-1-yl, cyclobutyl, cyclopentyl, cyclohexyl, 1-hydroxy-cyclopropyl, 1-hydroxy-cyclobutyl, 1-hydroxy-cyclopentyl, 1-hydroxy-cyclohexyl, ethinyl, ethoxy, difluoromethyl, trifluoromethyl, pentafluoroethyl, 2-hydroxyl-ethyl, hydroxymethyl, 3-hydroxy-propyl, 2-hydroxy-2-methyl-prop-1-yl, 3-hydroxy-3-methyl-but-1-yl, 1-hydroxy-1-methyl-ethyl, 2,2,2-trifluoro-1-hydroxy-1-methyl-ethyl, 2,2,2-trifluoro-1-hydroxy-1-trifluoromethyl-ethyl, 2-methoxy-ethyl, 2-ethoxy-ethyl, hydroxy, difluoro-methyloxy, trifluoromethyloxy, 2-methyloxy-ethyloxy, methylsulfanyl, methylsulfinyl, methlysulfonyl, ethylsulfinyl, ethylsulfonyl, trimethylsilyl, (R)-tetrahydrofuran-3-yloxy or (S)-tetra-hydrofuran-3-yloxy or cyano;
wherein R³ is preferably selected from cyclopropyl, ethyl, ethinyl, ethoxy, (R)-tetrahydrofuran-3-yloxy or (S)-tetrahydrofuran-3-yloxy; and most preferably R³ is cyclopropyl,
or a derivative thereof wherein one or more hydroxyl groups of the β-D-glucopyranosyl group are acylated with groups selected from (C₁₋₁₈-alkyl)carbonyl, (C₁₋₁₈-alkyl)oxycarbonyl, phenyl-carbonyl and phenyl-(C₁₋₃-alkyl)-carbonyl;
(2) Velagliflozin, represented by formula (2):

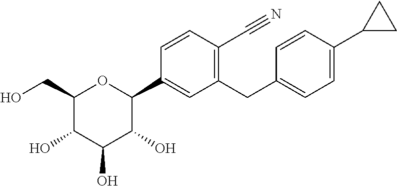 [SMILES: N#Cc1ccc([C@@H]2O[C@H](CO)[C@@H](O)[C@H](O)[C@H]2O)cc1Cc1ccc(C2CC2)cc1]

(3) Dapagliflozin, represented by formula (3):

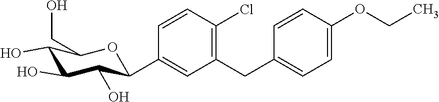

(4) Canagliflozin, represented by formula (4):

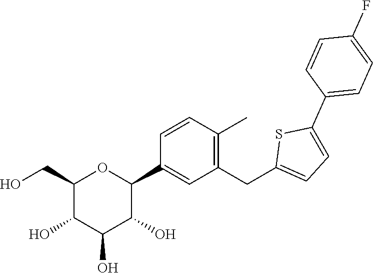 [SMILES: Cc1ccc([C@@H]2O[C@H](CO)[C@@H](O)[C@H](O)[C@H]2O)cc1Cc1ccc(-c2ccc(F)cc2)s1]

(5) Empagliflozin, represented by formula (5):

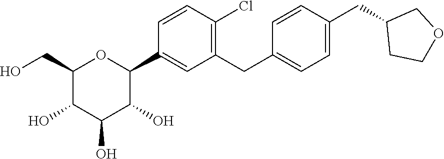 [SMILES: OC[C@H]1O[C@@H](c2ccc(Cl)c(Cc3ccc(C[C@H]4CCOC4)cc3)c2)[C@H](O)[C@@H](O)[C@@H]1O]

(6) Luseogliflozin, represented by formula (6):

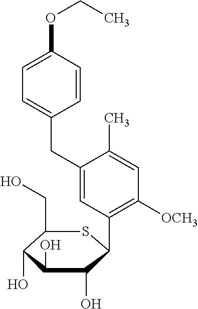

(7) Tofogliflozin, represented by formula (7):

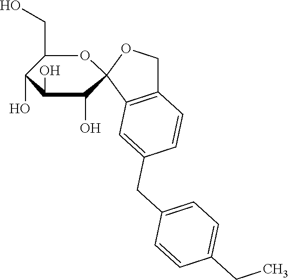

(8) Ipragliflozin, represented by formula (8):

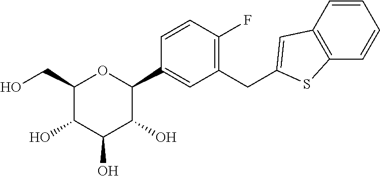 [SMILES: OC[C@H]1O[C@@H](c2ccc(F)c(Cc3cc4ccccc4s3)c2)[C@H](O)[C@@H](O)[C@@H]1O]

(9) Ertugliflozin, represented by formula (9):

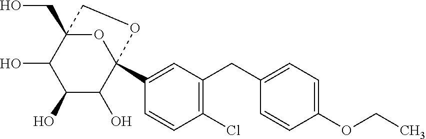 [SMILES: CCOc1ccc(Cc2cc([C@]34OC[C@](CO)(O3)C(O)[C@H](O)C4O)ccc2Cl)cc1]

(10) Atigliflozin, represented by formula (10):

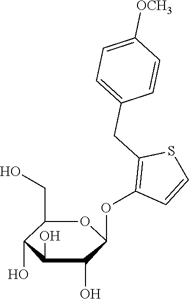

(11) Remogliflozin, represented by formula (11):

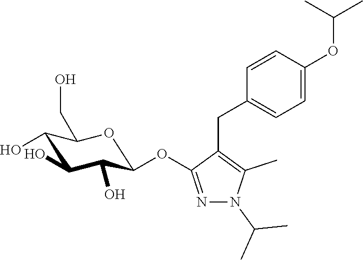

(11A) Remogliflozin etabonate, represented by formula (11A):

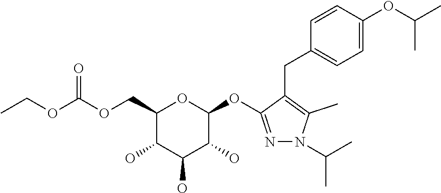 [SMILES: CCOC(=O)OC[C@H]1O[C@@H](Oc2nn(C(C)C)c(C)c2Cc2ccc(OC(C)C)cc2)[C@H](O)[C@@H](O)[C@@H]1O]

(12) a thiophene derivative of the formula (12)

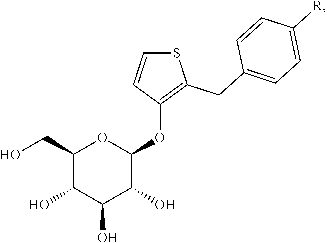

wherein R denotes methoxy or trifluoromethoxy;
(13) 1-(β-D)-glucopyranosyl)-4-methyl-3-15-(4-fluorophenyl)-2-thienylmethyl |benzene. represented by formula (13);

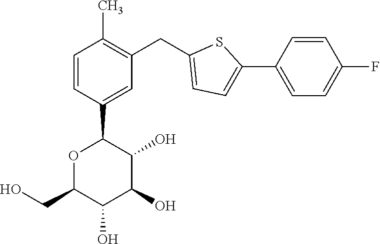 [SMILES: Cc1ccc([C@@H]2O[C@H](CO)[C@@H](O)[C@H](O)[C@H]2O)cc1Cc1ccc(-c2ccc(F)cc2)s1]

(14) a spiroketal derivative of the formula (14):

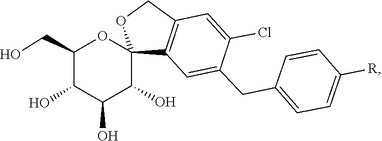

wherein R denotes methoxy, trifluoromethoxy, ethoxy, ethyl, isopropyl or tert. butyl;
(15) a pyrazole-O-glucoside derivative of the formula (15)

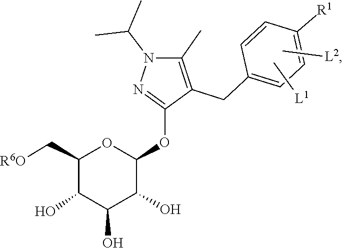

wherein
R¹ denotes C₁₋₃-alkoxy,
L¹, L² independently of each other denote H or F,
R⁶ denotes H, (C₁₋₃-alkyl)carbonyl, (C₁₋₆-alkyl)oxycarbonyl, phenyloxycarbonyl, benzyloxycarbonyl or benzylcarbonyl;
(16) Sotagliflozin, represented by formula (16):

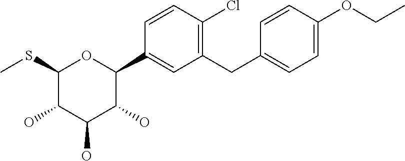 [SMILES: CCOc1ccc(Cc2cc([C@@H]3O[C@H](SC)[C@@H](O)[C@H](O)[C@H]3O)ccc2Cl)cc1]

(17) Sergliflozin, represented by formula (17):

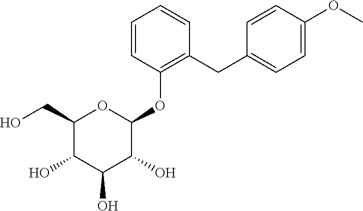 [SMILES: COc1ccc(Cc2ccccc2O[C@@H]2O[C@H](CO)[C@@H](O)[C@H](O)[C@H]2O)cc1]

(18) a compound represented by formula (18):

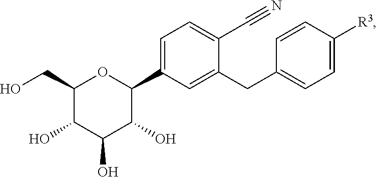

wherein
R³ denotes cyclopropyl, hydrogen, fluorine, chlorine, bromine, iodine, methyl, ethyl, propyl, isopropyl, butyl, sec-butyl, iso-butyl, tert-butyl, 3-methyl-but-1-yl, cyclobutyl, cyclopentyl, cyclohexyl, 1-hydroxy-cyclopropyl, 1-hydroxy-cyclobutyl, 1-hydroxy-cyclopentyl, 1-hydroxy-cyclohexyl, ethinyl, ethoxy, difluoromethyl, trifluoromethyl, pentafluoroethyl, 2-hydroxyl-ethyl, hydroxymethyl, 3-hydroxy-propyl, 2-hydroxy-2-methyl-prop-1-yl, 3-hydroxy-3-methyl-but-1-yl, 1-hydroxy-1-methyl-ethyl, 2,2,2-trifluoro-1-hydroxy-1-methyl-ethyl, 2,2,2-trifluoro-1-hydroxy-1-trifluoromethyl-ethyl, 2-methoxy-ethyl, 2-ethoxy-ethyl, hydroxy, difluoromethyloxy, trifluoromethyloxy, 2-methyloxy-ethyloxy, methylsulfanyl, methylsulfinyl, methlysulfonyl, ethylsulfinyl, ethylsulfonyl, trimethylsilyl, (R)-tetrahydrofuran-3-yloxy or (S)-tetrahydrofuran-3-yloxy or cyano, and wherein R³ is preferably selected from cyclopropyl, ethyl, ethinyl, ethoxy, (R)-tetrahydrofuran-3-yloxy or (S)-tetrahydrofuran-3-yloxy; and R³ most preferably is cyclopropyl,
or a derivative thereof wherein one or more hydroxyl groups of the β-D-glucopyranosyl group are acylated with groups selected from (C₁₋₁₈-alkyl)carbonyl, (C₁₋₁₈-alkyl)oxycarbonyl, phenylcarbonyl and phenyl-(C₁₋₃-alkyl)-carbonyl;
(19) Bexagliflozin, represented by formula (19):

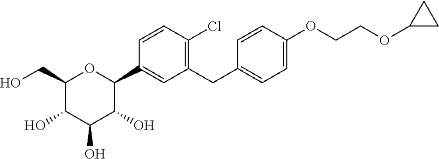 [SMILES: OC[C@H]1O[C@@H](c2ccc(Cl)c(Cc3ccc(OCCOC4CC4)cc3)c2)[C@H](O)[C@@H](O)[C@@H]1O]

(20) Janagliflozin, represented by formula (20):

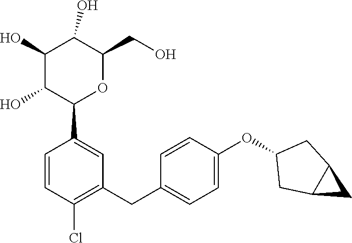 [SMILES: OC[C@H]1O[C@@H](c2ccc(Cl)c(Cc3ccc(O[C@H]4C[C@@H]5C[C@@H]5C4)cc3)c2)[C@H](O)[C@@H](O)[C@@H]1O]

(21) Rongliflozin, represented by formula (21):

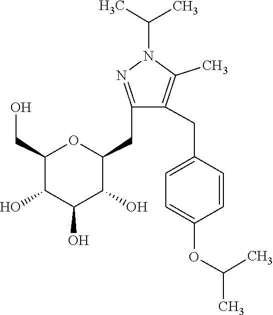 [SMILES: Cc1c(Cc2ccc(OC(C)C)cc2)c(C[C@@H]2O[C@H](CO)[C@@H](O)[C@H](O)[C@H]2O)nn1C(C)C]

(22) Wanpagliflozin;
(23) Enavogliflozin, represented by formula (23):

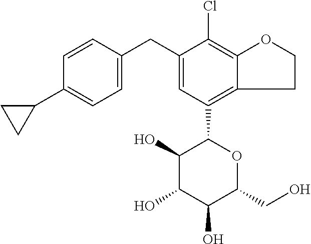 [SMILES: OC[C@H]1O[C@@H](c2cc(Cc3ccc(C4CC4)cc3)c(Cl)c3c2CCO3)[C@H](O)[C@@H](O)[C@@H]1O]

(24) TFC-039, represented by formula (24):

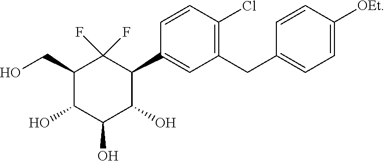 [SMILES: CCOc1ccc(Cc2cc([C@H]3[C@H](O)[C@@H](O)[C@H](O)[C@@H](CO)C3(F)F)ccc2Cl)cc1]

8. The one or more SGLT-2 inhibitors or pharmaceutically acceptable forms thereof in combination with pimobendan and/or telmisartan or pharmaceutically acceptable forms thereof for use according to any one of clauses 1 to 7, wherein the pharmaceutically acceptable form thereof is a crystalline complex between the one or more SGLT-2 inhibitors and one or more amino acids, preferably proline, more preferably L-proline; and most preferably is co-crystal of the one or more SGLT2 inhibitors, L-proline and crystalline water.
9. The one or more SGLT-2 inhibitors or pharmaceutically acceptable forms thereof in combination with pimobendan and/or telmisartan or pharmaceutically acceptable forms thereof for use according to any one of clauses 1 to 8, wherein velagliflozin or pharmaceutically acceptable forms thereof is to be administered as single SGLT-2 inhibitor in combination with pimobendan and/or telmisartan or pharmaceutically acceptable forms thereof; preferably wherein velagliflozin or pharmaceutically acceptable forms thereof is to be administered as single SGLT-2 inhibitor in combination with pimobendan.
10. The one or more SGLT-2 inhibitors or pharmaceutically acceptable forms thereof in combination with pimobendan and/or telmisartan or pharmaceutically acceptable forms thereof for use according to any one of clauses 1 to 9, wherein velagliflozin or pharmaceutically acceptable forms thereof is used as single SGLT-2 inhibitor, and wherein the use is in a method of prevention and/or treatment of DCM or MVD in a canine/canine patient.
11. The one or more SGLT-2 inhibitors or pharmaceutically acceptable forms thereof in combination with pimobendan and/or telmisartan or pharmaceutically acceptable forms thereof for use according to any one of clauses 1 to 9, wherein velagliflozin or pharmaceutically acceptable forms thereof is used as single SGLT-2 inhibitor, and wherein the use is in a method of prevention and/or treatment of HCM in a feline/feline patient.
12. The one or more SGLT-2 inhibitors or pharmaceutically acceptable forms thereof in combination with pimobendan and/or telmisartan or pharmaceutically acceptable forms thereof for use according to any one of clauses 1 to 9, wherein velagliflozin or pharmaceutically acceptable forms thereof is used as single SGLT-2 inhibitor, and wherein the use is in a method of treatment of DCM or MVD in a canine/canine patient.
13. The one or more SGLT-2 inhibitors or pharmaceutically acceptable forms thereof in combination with pimobendan and/or telmisartan or pharmaceutically acceptable forms thereof for use according to any one of clauses 1 to 9, wherein velagliflozin or pharmaceutically acceptable forms thereof is used as single SGLT-2 inhibitor, and wherein the use is in a method of treatment of HCM in a feline/feline patient.
14. The one or more SGLT-2 inhibitors or pharmaceutically acceptable forms thereof in combination with pimobendan and/or telmisartan or pharmaceutically acceptable forms thereof for use according to any one of clauses 1 to 9, wherein velagliflozin or pharmaceutically acceptable forms thereof is used as single SGLT-2 inhibitor, and wherein the use is in combination with pimobendan in a method of treatment of DCM or MVD in a canine/canine patient.
15. The one or more SGLT-2 inhibitors or pharmaceutically acceptable forms thereof in combination with pimobendan and/or telmisartan or pharmaceutically acceptable forms thereof for use according to any one of clauses 1 to 9, wherein velagliflozin or pharmaceutically acceptable forms thereof is used as single SGLT-2 inhibitor, and wherein the use is in combination with pimobendan in a method of treatment of HCM in a feline/feline patient.
16. The one or more SGLT-2 inhibitors or pharmaceutically acceptable forms thereof in combination with pimobendan and/or telmisartan or pharmaceutically acceptable forms thereof for use according to any one of clauses 1 to 15, wherein the one or more SGLT-2 inhibitors or pharmaceutically acceptable forms thereof are administered orally, parenterally, intravenously, subcutaneously or intramuscularly, preferably orally.
17. The one or more SGLT-2 inhibitors or pharmaceutically acceptable forms thereof in combination with pimobendan and/or telmisartan or pharmaceutically acceptable forms thereof for use according to any one of clauses 1 to 16, wherein the one or more SGLT-2 inhibitors or pharmaceutically acceptable forms thereof are to be administered at a dose of 0.01 mg/kg bodyweight to 10 mg/kg bodyweight per day, preferably at a dose of 0.01 mg/kg bodyweight to 5 mg/kg bodyweight per day, more preferably at a dose of 0.01 mg/kg bodyweight to 4 mg/kg bodyweight per day, even more preferably at a dose of 0.01 mg/kg bodyweight to 3 mg/kg bodyweight per day, even more preferably at a dose of 0.01 mg/kg bodyweight to 2 mg/kg bodyweight per day, even more preferably at a dose of 0.01 mg/kg bodyweight to 1 mg/kg bodyweight per day, even more preferably at a dose of 0.01 mg/kg bodyweight to 0.5 mg/kg bodyweight per day, most preferably at a dose of 0.01 mg/kg bodyweight to 0.3 mg/kg bodyweight per day.
18. The one or more SGLT-2 inhibitors or pharmaceutically acceptable forms thereof in combination with pimobendan and/or telmisartan or pharmaceutically acceptable forms thereof for use according to any one of clauses 1 to 17, wherein such one or more SGLT-2 inhibitors or pharmaceutically acceptable forms thereof is to be administered once per day or twice per day, preferably once per day.
19. The one or more SGLT-2 inhibitors or pharmaceutically acceptable forms thereof in combination with pimobendan and/or telmisartan or pharmaceutically acceptable forms thereof for use according to any one of clauses 1 to 18, wherein pimobendan is to be administered at a dose of 0.1 mg/kg bodyweight to 1 mg/kg bodyweight per day, preferably 0.2 mg/kg bodyweight to 0.6 mg/kg bodyweight per day, more preferably 0.5 mg/kg bodyweight per day.
20. The one or more SGLT-2 inhibitors or pharmaceutically acceptable forms thereof in combination with pimobendan and/or telmisartan or pharmaceutically acceptable forms thereof for use according to any one of clauses 1 to 19, wherein pimobendan is to be administered once per day or twice per day, preferably twice a day, more preferably every twelve hours.
21. The one or more SGLT-2 inhibitors or pharmaceutically acceptable forms thereof in combination with pimobendan and/or telmisartan or pharmaceutically acceptable forms thereof for use according to any one of clauses 1 to 20, wherein the one or more SGLT-2 inhibitors or pharmaceutically acceptable forms thereof are administered in combination with pimobendan and telmisartan or pharmaceutically acceptable forms thereof, preferably wherein the one or more SGLT-2 inhibitors or pharmaceutically acceptable forms thereof are to be administered before, after or concomitantly with administering pimobendan and telmisartan or pharmaceutically acceptable forms thereof.
22. The one or more SGLT-2 inhibitors or pharmaceutically acceptable forms thereof in combination with pimobendan and/or telmisartan or pharmaceutically acceptable forms thereof for use according to any one of clauses 1 to 21, wherein telmisartan or pharmaceutically acceptable forms thereof is to be administered at a dose of 0.01 to 10 mg/kg of bodyweight per day, preferably 0.05 to 8 mg/kg of bodyweight per day, even more preferably 0.1 to 5 mg/kg of bodyweight per day, even more preferably 0.2 to 4 mg/kg of bodyweight per day, even more preferably 0.3 to 3 mg/kg of bodyweight per day, even more preferably 0.4 to 2.5 mg/kg of bodyweight per day, even more preferably 0.5 to 2 mg/kg of bodyweight per day, most preferably 0.75 to 1.5 mg/kg of bodyweight per day.
23. The one or more SGLT-2 inhibitors or pharmaceutically acceptable forms thereof in combination with pimobendan and/or telmisartan or pharmaceutically acceptable forms thereof for use according to any one of clauses 1 to 22, wherein telmisartan or pharmaceutically acceptable forms thereof is to be administered once per day or twice per day, preferably twice a day, more preferably every twelve hours.
24. The one or more SGLT-2 inhibitors or pharmaceutically acceptable forms thereof in combination with pimobendan and/or telmisartan or pharmaceutically acceptable forms thereof for use according to any one of clauses 1 to 23, wherein the preventive and/or therapeutic effect is characterized by one or more of the following clinical and/or biochemical parameters:
   improved cardiometabolic efficiency, characterized by an increased ratio of [cardiac output/metabolic substrate consumed] and/or characterized by an increased ratio of [cardiac output/oxygen consumed];
   increase of the production of ketone bodies in the liver, characterized by increased plasma levels of 3-hydroxy butyric acid and/or the corresponding acylcarnitines i.e., hydroxybutyry lcarnitine and increased plasma levels of one or more of the branched-chain amino acids (valine, leucine and isoleucine);
   improved cardiac function by achieved reduced pre-and/or afterload, improved arterial wall structure function:
   improved echocardiographic parameters, such as decreased LA (Left atrium dimension measured as right parasternal short-axis), LA/Ao (left atrium to aorta ratio; Ao=Aortic root diameter), IVSd (interventricular septal end diastolic dimension, i.e. the thickness of the interventricular septum), and/or LAD (Left atrium measured as right parasternal long-axis), and improved cardiac biomarkers, such as decreased NT-proBNP (N-terminal prohormone of brain natriuretic peptide) and/or decreased cTnI (cardiac Troponin I) and/or increased erythropoietin concentration and/or changes of metabolites indicating a change of cardiac energy sources; as well as improved heart murmur;
   delayed onset of different phenotypes of cardiac diseases, such as (M)MVD and/or DCM and/or cardiomyopathies, preferably at least by 1, 2, 3, 4, 5, 6, 7, 8, 9, 10, 11, 12, 13, 14, 15 or more months, or even stopped progression of different phenotypes of cardiac diseases, such as (M)MVD and/or DCM and/or cardiomyopathies;
   longer time of survival, preferably at least by 1, 2, 3, 4, 5, 6, 7, 8, 9, 10, 11, 12, 13, 14, 15 or more months, and/or delay of next episode of heart failure, preferably at least by 1, 2, 3, 4, 5, 6, 7, 8, 9, 10, 11, 12, 13, 14, 15 or more months, and/or lower level of cardiac mortality and/or morbidity;
   improved clinical signs, such as reduced e.g. breathlessness or dyspnea, cough, depression, exercise intolerance, inappetence, syncope, abdominal distention and/or polydipsia;
   prolongation of time to event (e.g., heart failure, cardiac death, onset of clinical signs, need for additional concomitant medication, increase in dose of concomitant therapy—diuretics);
   prevention of fibrosis;
   prevention of cardiomyocytes death/protection of cardiomyocytes (reduction of oxidative stress);
   increase of vascular wall flexibility:;
   prevention of hypertension;
   higher quality of life.
25. A pharmaceutical composition comprising one or more SGLT2 inhibitors or pharmaceutically acceptable forms thereof in combination with pimobendan and/or telmisartan or pharmaceutically acceptable forms thereof for use as defined in any one of clauses 1 to 24, wherein preferably such pharmaceutical composition is a fixed-dose-combination (FDC) of the one or more SGLT-2 inhibitors or pharmaceutically acceptable forms thereof and pimobendan and/or telmisartan or pharmaceutically acceptable forms thereof, wherein more preferably such FDC is a solid or a liquid formulation.

1. A method of prevention and/or treatment of one or more cardiac diseases in a non-human mammal comprising administering to the non-human mammal a pharmaceutical composition comprising one or more SGLT-2 inhibitors or pharmaceutically acceptable forms thereof in combination with pimobendan and/or telmisartan or pharmaceutically acceptable forms thereof.

2. The method of claim 1, wherein the non-human mammal is a canine or a feline.

3. (canceled)

4. The method of claim 1, wherein the one or more cardiac diseases is selected from the group consisting of acquired cardiac disease: congenital cardiac disease; heart failure; congestive heart failure: asymptomatic/preclinical/occult heart failure; heart failure due to (myxomatous) mitral valve disease [(M)MVD]; congestive heart failure due to (myxomatous) mitral valve disease [(M)MVD]; asymptomatic/preclinical/occult heart failure due to (myxomatous) mitral valve disease [(M)MVD]; (myxomatous) mitral valve disease [(M)MVD]; clinically overt (myxomatous) mitral valve disease [(M)MVD]; asymptomatic/preclinical/occult (myxomatous) mitral valve disease [(M)MVD]; heart failure due to dilated cardiomyopathy (DCM); congestive heart failure due to dilated cardiomyopathy (DCM); asymptomatic/preclinical/occult heart failure due to dilated cardiomyopathy (DCM); dilated cardiomyopathy (DCM); clinically overt dilated cardiomyopathy (DCM); asymptomatic/preclinical/occult dilated cardiomyopathy (DCM); aortic stenosis (valvular, supravalvular and/or subvalvular); heart failure due to one or more cardiomyopathies: heart failure due to hypertrophic cardiomyopathy (HCM); heart failure due to hypertrophic obstructive cardiomyopathy (HOCM); heart failure due to restrictive cardiomyopathy (RCM); heart failure due to dilated cardiomyopathy (DCM); heart failure due to unclassified cardiomyopathy (UCM); heart failure due to arrhythmogenic right ventricular cardiomyopathy (ARVC); hypertrophic cardiomyopathy (HCM); hypertrophic obstructive cardiomyopathy (HOCM); restrictive cardiomyopathy (RCM); dilated cardiomyopathy (DCM); unclassified cardiomyopathy (UCM); and arrhythmogenic right ventricular cardiomyopathy (ARVC).

5. The method of claim 1, wherein:
the one or more cardiac diseases is selected from the group consisting of acquired cardiac disease: congenital cardiac disease; heart failure; congestive heart failure; asymptomatic/preclinical/occult heart failure; (myxomatous) mitral valve disease [(M)MVD]; clinically overt (myxomatous) mitral valve disease [(M)MVD]; asymptomatic/preclinical/occult (myxomatous) mitral valve disease [(M)MVD]; dilated cardiomyopathy (DCM); clinically overt dilated cardiomyopathy (DCM); asymptomatic/preclinical/occult dilated cardiomyopathy (DCM); aortic stenosis (valvular. supravalvular and/or subvalvular); and
the non-human mammal is a canine.

6. The method of claim 1, wherein the one or more cardiac diseases are selected from the group consisting of: acquired cardiac disease: congenital cardiac disease; heart failure; congestive heart failure; asymptomatic/preclinical/occult heart failure: heart failure due to one or more cardiomyopathies: heart failure due to hypertrophic cardiomyopathy (HCM); heart failure due to hypertrophic obstructive cardiomyopathy (HOCM); heart failure due to restrictive cardiomyopathy (RCM); heart failure due to dilated cardiomyopathy (DCM); heart failure due to unclassified cardiomyopathy (UCM); heart failure due to arrhythmogenic right ventricular cardiomyopathy (ARVC); hypertrophic cardiomyopathy (HCM); hypertrophic obstructive cardiomyopathy (HOCM); restrictive cardiomyopathy (RCM); dilated cardiomyopathy (DCM); unclassified cardiomyopathy (UCM); and/or arrhythmogenic right ventricular cardiomyopathy (ARVC); and wherein the non-human mammal/non-human mammal patient is a feline/feline patient; preferably a feline patient in need of such prevention and/or treatment, more preferably a cat in need of such prevention and/or treatment, even more preferably a non-diabetic cat in need of such prevention and/or treatment.

7. The method of claim 1, wherein the one or more SGLT-2 inhibitors is selected from the group consisting of:
(1) a glucopyranosyl-substituted benzene derivative of the formula (1) 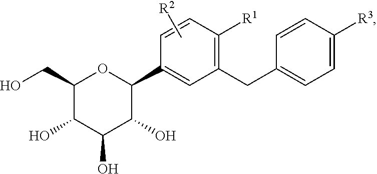
wherein R¹ denotes cyano, Cl or methyl,
R² denotes H, methyl, methoxy or hydroxy, and
R³ denotes cyclopropyl, hydrogen, fluorine, chlorine, bromine, iodine, methyl, ethyl, propyl, isopropyl, butyl, sec-butyl, iso-butyl, tert-butyl, 3-methyl-but-1-yl, cyclobutyl, cyclopentyl, cyclohexyl, 1-hydroxy-cyclopropyl, 1-hydroxy-cyclobutyl, 1-hydroxy-cyclopentyl, 1-hydroxy-cyclohexyl, ethinyl, ethoxy, difluoromethyl, trifluoromethyl, pentafluoroethyl, 2-hydroxyl-ethyl, hydroxymethyl, 3-hydroxy-propyl, 2-hydroxy-2-methyl-prop-1-yl, 3-hydroxy-3-methyl-but-1-yl, 1-hydroxy-1-methyl-ethyl, 2,2,2-trifluoro-1-hydroxy-1-methyl-ethyl, 2,2,2-trifluoro-1-hydroxy-1-trifluoromethyl-ethyl, 2-methoxy-ethyl, 2-ethoxy-ethyl, hydroxy, difluoromethyloxy, trifluoromethyloxy, 2-methyloxy-ethyloxy, methylsulfanyl, methylsulfinyl, methlysulfonyl, ethylsulfinyl, ethylsulfonyl, trimethylsilyl, (R)-tetrahydrofuran-3-yloxy or (S)-tetrahydrofuran-3-yloxy or cyano;
or a derivative thereof wherein one or more hydroxyl groups of the β-D-glucopyranosyl group are acylated with groups selected from (C₁₋₁₈-alkyl)carbonyl, (C₁₋₁₈-alkyl)oxycarbonyl, phenylcarbonyl and phenyl-(C₁₋₃-alkyl)-carbonyl;
(2) Velagliflozin, represented by formula (2): 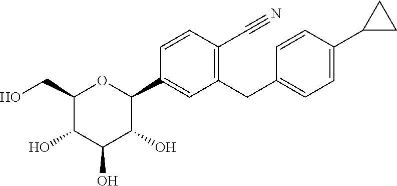 [SMILES: N#Cc1ccc([C@@H]2O[C@H](CO)[C@@H](O)[C@H](O)[C@H]2O)cc1Cc1ccc(C2CC2)cc1]
(3) Dapagliflozin, represented by formula (3): 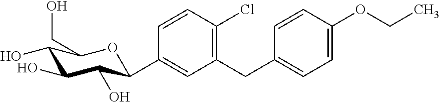
(4) Canagliflozin, represented by formula (4): 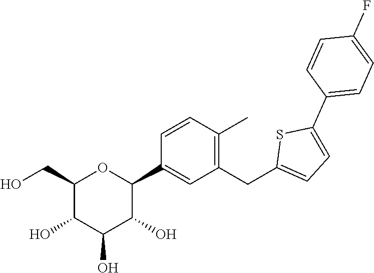 [SMILES: Cc1ccc([C@@H]2O[C@H](CO)[C@@H](O)[C@H](O)[C@H]2O)cc1Cc1ccc(-c2ccc(F)cc2)s1]
(5) Empagliflozin, represented by formula (5): 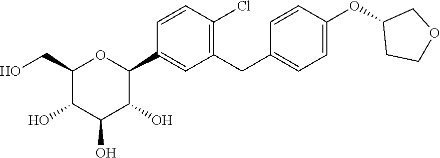 [SMILES: OC[C@H]1O[C@@H](c2ccc(Cl)c(Cc3ccc(O[C@H]4CCOC4)cc3)c2)[C@H](O)[C@@H](O)[C@@H]1O]
(6) Luseogliflozin, represented by formula (6): 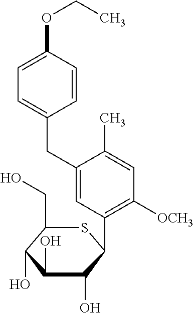
(7) Tofogliflozin, represented by formula (7): 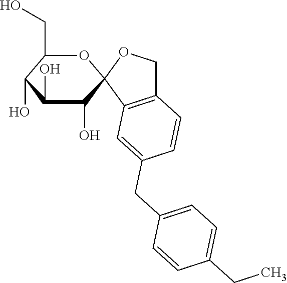
(8) Ipragliflozin, represented by formula (8): 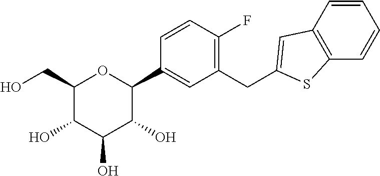 [SMILES: OC[C@H]1O[C@@H](c2ccc(F)c(Cc3cc4ccccc4s3)c2)[C@H](O)[C@@H](O)[C@@H]1O]
(9) Ertugliflozin, represented by formula (9): 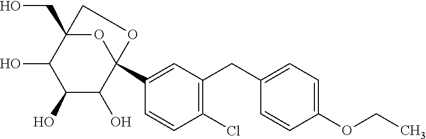 [SMILES: CCOc1ccc(Cc2cc([C@]34OC[C@](CO)(O3)C(O)[C@H](O)C4O)ccc2Cl)cc1]
(10) Atigliflozin, represented by formula (10): 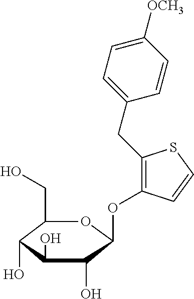
(11) Remogliflozin, represented by formula (11): 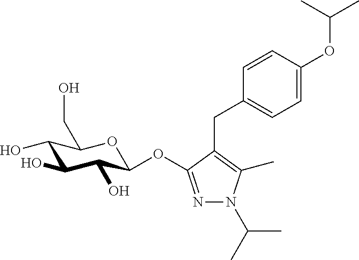
(11A) Remogliflozin etabonate, represented by formula (11A): 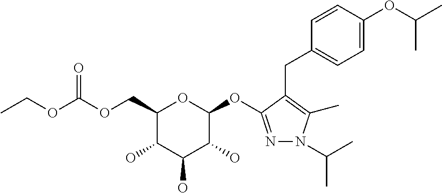 [SMILES: CCOC(=O)OC[C@H]1O[C@@H](Oc2nn(C(C)C)c(C)c2Cc2ccc(OC(C)C)cc2)[C@H](O)[C@@H](O)[C@@H]1O]
(12) a thiophene derivative of the formula (12) 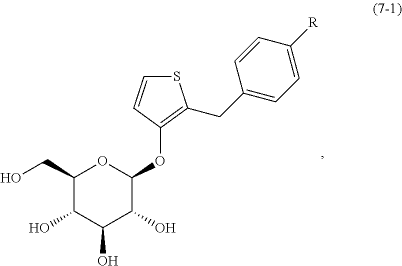
wherein R denotes methoxy or trifluoromethoxy;
(13) 1-(β-D-glucopyranosyl)-4-methyl-3-[5-(4-fluorophenyl)-2-thienylmethyl]benzene, represented by formula (13); 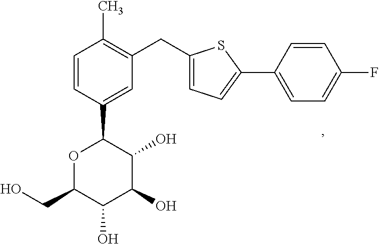 [SMILES: Cc1ccc([C@@H]2O[C@H](CO)[C@@H](O)[C@H](O)[C@H]2O)cc1Cc1ccc(-c2ccc(F)cc2)s1]
(14) a spiroketal derivative of the formula (14): 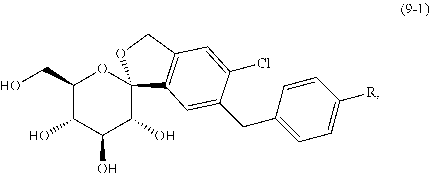
wherein R denotes methoxy, trifluoromethoxy, ethoxy, ethyl, isopropyl or tert. butyl;
(15) a pyrazole-O-glucoside derivative of the formula (15) 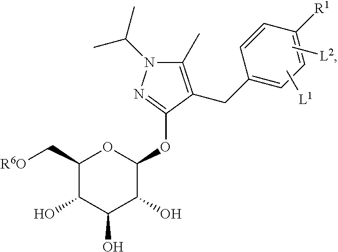
wherein
R¹ denotes C₁₋₃-alkoxy,
L¹, L² independently of each other denote H or F,
R⁶ denotes H, (C₁₋₃-alkyl)carbonyl, (C₁₋₆-alkyl)oxycarbonyl, phenyloxycarbonyl, benzyloxycarbonyl or benzylcarbonyl;
(16) Sotagliflozin, represented by formula (16): 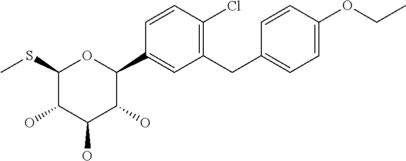 [SMILES: CCOc1ccc(Cc2cc([C@@H]3O[C@H](SC)[C@@H](O)[C@H](O)[C@H]3O)ccc2Cl)cc1]
(17) Sergliflozin, represented by formula (17): 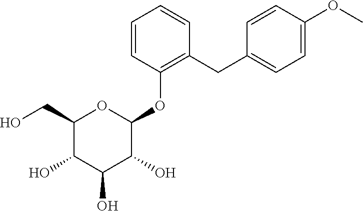 [SMILES: COc1ccc(Cc2ccccc2O[C@@H]2O[C@H](CO)[C@@H](O)[C@H](O)[C@H]2O)cc1]
(18) a compound represented by formula (18): 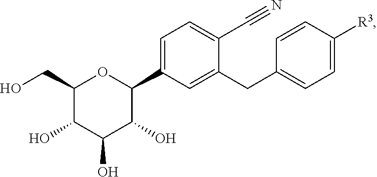
wherein
R³ denotes cyclopropyl, hydrogen, fluorine, chlorine, bromine, iodine, methyl, ethyl, propyl, isopropyl, butyl, sec-butyl, iso-butyl, tert-butyl, 3-methyl-but-1-yl, cyclobutyl, cyclopentyl, cyclohexyl, 1-hydroxy-cyclopropyl, 1-hydroxy-cyclobutyl, 1-hydroxy-cyclopentyl, 1-hydroxy-cyclohexyl, ethinyl, ethoxy, difluoromethyl, trifluoromethyl, pentafluoroethyl, 2-hydroxyl-ethyl, hydroxymethyl, 3-hydroxy-propyl, 2-hydroxy-2-methyl-prop-1-yl, 3-hydroxy-3-methyl-but-1-yl, 1-hydroxy-1-methyl-ethyl, 2,2,2-trifluoro-1-hydroxy-1-methyl-ethyl, 2,2,2-trifluoro-1-hydroxy-1-trifluoromethyl-ethyl, 2-methoxy-ethyl, 2-ethoxy-ethyl, hydroxy, difluoromethyloxy, trifluoromethyloxy, 2-methyloxy-ethyloxy, methylsulfanyl, methylsulfinyl, methlysulfonyl, ethylsulfinyl, ethylsulfonyl, trimethylsilyl, (R)-tetrahydrofuran-3-yloxy or (S)-tetrahydrofuran-3-yloxy or cyano,
or a derivative thereof wherein one or more hydroxyl groups of the β-D-glucopyranosyl group are acylated with groups selected from (C₁₋₁₈-alkyl)carbonyl, (C₁₋₁₈-alkyl)oxycarbonyl, phenylcarbonyl and phenyl-(C₁₋₃-alkyl)-carbonyl;
(19) Bexagliflozin, represented by formula (19): 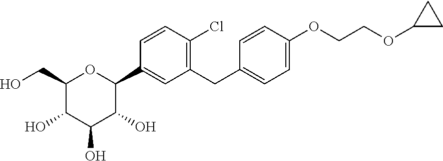 [SMILES: OC[C@H]1O[C@@H](c2ccc(Cl)c(Cc3ccc(OCCOC4CC4)cc3)c2)[C@H](O)[C@@H](O)[C@@H]1O]
(20) Janagliflozin, represented by formula (20): 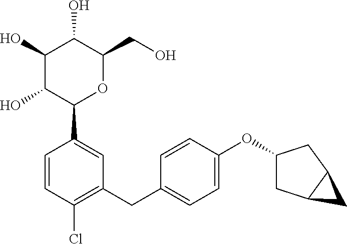 [SMILES: OC[C@H]1O[C@@H](c2ccc(Cl)c(Cc3ccc(O[C@H]4C[C@@H]5C[C@@H]5C4)cc3)c2)[C@H](O)[C@@H](O)[C@@H]1O]
(21) Rongliflozin, represented by formula (21): 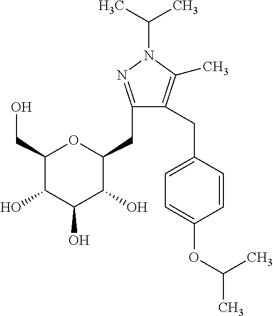 [SMILES: Cc1c(Cc2ccc(OC(C)C)cc2)c(C[C@@H]2O[C@H](CO)[C@@H](O)[C@H](O)[C@H]2O)nn1C(C)C]
(22) Wanpagliflozin;
(23) Enavogliflozin, represented by formula (23): 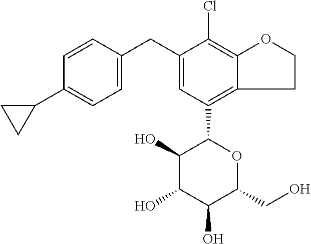 [SMILES: OC[C@H]1O[C@@H](c2cc(Cc3ccc(C4CC4)cc3)c(Cl)c3c2CCO3)[C@H](O)[C@@H](O)[C@@H]1O]
(24) TFC-039, represented by formula (24):

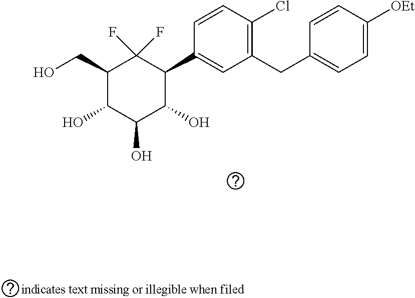 [SMILES: CCOc1ccc(Cc2cc([C@H]3[C@H](O)[C@@H](O)[C@H](O)[C@@H](CO)C3(F)F)ccc2Cl)cc1]

8. The method of claim 1, wherein the pharmaceutically acceptable form thereof is a crystalline complex between the one or more SGLT-2 inhibitors and one or more amino acids, such as proline.

9. The method of claim 1, wherein velagliflozin or a pharmaceutically acceptable form thereof is administered as a single SGLT-2 inhibitor in combination with pimobendan and/or telmisartan or pharmaceutically acceptable forms thereof.

10. The method of claim 1, wherein the non-human mammal is a canine, and the method further comprises prevention and/or treatment of DCM or MVD in the canine by administering to the canine a single SGLT-2 inhibitor consisting of velagliflozin or a pharmaceutically acceptable form thereof in combination with pimobendan and/or telmisartan.

11. The method of claim 1, wherein the non-human mammal is a feline, and the method further comprises prevention and/or treatment of HCM in the feline by administering to the feline a single SLGT-2 inhibitor consisting of velagliflozin or a pharmaceutically acceptable form thereof in combination with pimobendan and/or telmisartan.

12. The method of claim 1, wherein the non-human mammal is a canine, and the method further comprises treatment of DCM or MVD in the canine by administering to the canine a single SLGT-2 inhibitor consisting of velagliflozin or a pharmaceutically acceptable form thereof in combination with pimobendan and/or telmisartan.

13. The method of claim 1, wherein the non-human mammal is a feline, and the method further comprises treatment of HCM in the feline by administering to the feline a single SGLT-2 inhibitor consisting of velagliflozin or a pharmaceutically acceptable form thereof in combination with pimobendan and/or telmisartan.

14. The method of claim 1, wherein the non-human mammal is a canine, and the method further comprises treatment of DCM or MVD in the canine by administering to the canine a single SGLT-2 inhibitor consisting of velagliflozin or a pharmaceutically acceptable form thereof in combination with pimobendan.

15. The method of claim 1, wherein the non-human mammal is a feline, and the method further comprises treatment of HCM in the feline by administering to the feline a single SGLT-2 inhibitor consisting of velagliflozin or a pharmaceutically acceptable form thereof in combination with pimobendan.

16. The method of claim 1, wherein the one or more SGLT-2 inhibitors or pharmaceutically acceptable forms thereof is administered orally. parenterally. intravenously. subcutaneously or intramuscularly. preferably orally.

17. The method of claim 1, wherein the one or more SGLT-2 inhibitors or pharmaceutically acceptable form thereof is administered at a dose of 0.01 mg/kg bodyweight to 10 mg/kg bodyweight per day, or at a dose of 0.01 mg/kg bodyweight to 5 mg/kg bodyweight per day, or at a dose of 0.01 mg/kg bodyweight to 4 mg/kg bodyweight per day, or at a dose of 0.01 mg/kg bodyweight to 3 mg/kg bodyweight per day, or at a dose of 0.01 mg/kg bodyweight to 2 mg/kg bodyweight per day, or at a dose of 0.01 mg/kg bodyweight to I mg/kg bodyweight per day, or at a dose of 0.01 mg/kg bodyweight to 0.5 mg/kg bodyweight per day, or at a dose of 0.01 mg/kg bodyweight to 0.3 mg/kg bodyweight per day.

18. The method of claim 1, wherein the one or more SGLT-2 inhibitors or pharmaceutically acceptable form thereof is administered once per day or twice per day.

19. The method of claim 1, wherein pimobendan is administered at a dose of 0.1 mg/kg bodyweight to 1 mg/kg bodyweight per day, or 0.2 mg/kg bodyweight to 0.6 mg/kg bodyweight per day, or 0.5 mg/kg bodyweight per day.

20. The method of claim 1, wherein pimobendan is administered once per day or twice per day, or twice a day, or every twelve hours.

21. The method of claim 1, wherein the one or more SGLT-2 inhibitors or pharmaceutically acceptable form thereof is administered in combination with pimobendan and telmisartan or pharmaceutically acceptable forms thereof, and the one or more SGLT-2 inhibitors or pharmaceutically acceptable form thereof is administered before. after or concomitantly with administering pimobendan and telmisartan or pharmaceutically acceptable forms thereof.

22. The method of claim 1, wherein telmisartan or pharmaceutically acceptable forms thereof is administered at a dose of 0.01 to 10 mg/kg of bodyweight per day, or 0.05 to 8 mg/kg of bodyweight per day, or 0. 1 to 5 mg/kg of bodyweight per day, or 0.2 to 4 mg/kg of bodyweight per day, or 0.3 to 3 mg/kg of bodyweight per day, or 0.4 to 2.5 mg/kg of bodyweight per day, or 0.5 to 2 mg/kg of bodyweight per day, or 0).75 to 1.5 mg/kg of bodyweight per day.

23. The method of claim 1, wherein telmisartan or a pharmaceutically acceptable form thereof is administered once per day or twice per day, or twice a day, or every twelve hours.

24. The method of claim 1, wherein the the method achieves a preventive and/or therapeutic effect for the non-human mammal thaty is characterized by one or more of the following clinical and/or biochemical parameters:
improved cardiometabolic efficiency. characterized by an increased ratio of [cardiac output/metabolic substrate consumed]and/or characterized by an increased ratio of [cardiac output/oxygen consumed];
increase of the production of ketone bodies in the liver, characterized by increased plasma levels of 3-hydroxy butyric acid and/or the corresponding acylcarnitines, i.e., hydroxy butyrylcarnitine, and increased plasma levels of one or more of the branched-chain amino acids (valine, leucine and isoleucine);
improved cardiac function by achieved reduced pre-and/or afterload, improved arterial wall structure function;
improved echocardiographic parameters, such as decreased LA (Left atrium dimension measured as right parasternal short-axis), LA/Ao (left atrium to aorta ratio; Ao=Aortic root diameter), IVSd (interventricular septal end diastolic dimension, i.e. the thickness of the interventricular septum), and/or LAD (Left atrium measured as right parasternal long-axis), and improved cardiac biomarkers, such as decreased NT-proBNP (N-terminal prohormone of brain natriuretic peptide) and/or decreased cTnI (cardiac Troponin I) and/or increased erythropoietin concentration and/or changes of metabolites indicating a change of cardiac energy sources as well as improved heart murmur;
delayed onset of different phenotypes of cardiac diseases, such as (M)MVD and/or DCM and/or cardiomyopathies, at least by 1, 2, 3, 4, 5, 6, 7, 8, 9, 10, 11, 12, 13, 14, 15 or more months, or even stopped progression of different phenotypes of cardiac diseases, such as (M)MVD and/or DCM and/or cardiomyopathies;
longer time of survival, at least by 1, 2, 3, 4, 5, 6, 7, 8, 9, 10, 11, 12, 13, 14, 15 or more months, and/or delay of next episode of heart failure, at least by 1. 2. 3, 4, 5, 6, 7, 8, 9, 10, 11, 12, 13, 14, 15 or more months, and/or lower level of cardiac mortality and/or morbidity;
improved clinical signs, such as reduced e.g., breathlessness or dyspnea, cough, depression, exercise intolerance, inappetence, syncope, abdominal distention and/or poly dipsia;
prolongation of time to event (e.g., heart failure, cardiac death, onset of clinical signs, need for additional concomitant medication, increase in dose of concomitant therapy—diuretics);
prevention of fibrosis;
prevention of cardiomyocytes death/protection of cardiomyocytes (reduction of oxidative stress);
increase of vascular wall flexibility;
prevention of hypertension; and
higher quality of life.

25. The method of claim 1, wherein the pharmaceutical composition is a fixed-dose-combination (FDC) of the one or more SGLT-2 inhibitors or pharmaceutically acceptable form thereof and pimobendan and/or telmisartan or pharmaceutically acceptable forms thereof, and the FDC is a solid formulation or a liquid formulation.
